(19) United States
(12) Patent Application Publication
(10) Pub. No.: US 2023/0365577 A1
(43) Pub. Date: Nov. 16, 2023

(54) Photodynamic Therapy and Diagnosis

(71) Applicant: RMW CHO Group Limited, Kwai Chung (HK)

(72) Inventors: Honsue Cho, Melbourne (AU); Andrew N. Stephens, Melbourne (AU); Sebastian M. Marcuccio, Melbourne (AU); Christopher D. Donner, Melbourne (AU); Kieran P. Stockton, Melbourne (AU); Colin Skene, Melbourne (AU); Jonathan Faber, Melbourne (AU)

(21) Appl. No.: 18/320,082

(22) Filed: May 18, 2023

Related U.S. Application Data
(63) Continuation of application No. PCT/EP2021/083253, filed on Nov. 26, 2021.

(30) Foreign Application Priority Data
Nov. 26, 2020 (GB) GB2018667.2

(51) Int. Cl. C07D 487/22; A61N 5/06; C07F 3/06; A61K 45/06

(52) U.S. Cl. CPC C07D 487/22; A61N 5/062; C07F 3/06; A61K 45/06; A61N 2005/0659; A61N 2005/0663

(57) ABSTRACT

The present invention relates to phyllochlorin analogues and their pharmaceutically acceptable salts, and compositions comprising phyllochlorin analogues and their pharmaceutically acceptable salts. Phyllochlorin analogues and pharmaceutically acceptable salts thereof are suitable for use in photodynamic therapy, cytoluminescent therapy and photodynamic diagnosis, for example, for treating or detecting a tumour, so or for antiviral treatment. The present invention also relates to the use of phyllochlorin analogues and pharmaceutically acceptable salts thereof in the manufacture of a phototherapeutic or photodiagnostic agent, and to a method of photodynamic therapy, cytoluminescent therapy or photodynamic diagnosis, for example, for treating or detecting a tumour, or for antiviral treatment.

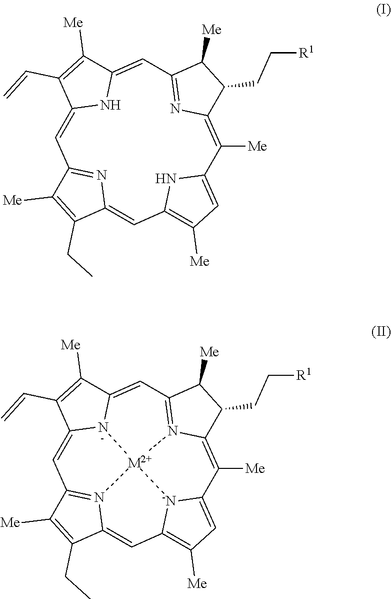

### RELATED APPLICATIONS

This application is a continuation of International PCT application no. PCT/EP2021/083253, filed Nov. 26, 2021, which claims priority to GB patent application no. 2018667.2, filed Nov. 26, 2020. The disclosures of the above-referenced applications are incorporated by reference herein in their entireties.

### TECHNICAL FIELD

The present invention relates to phyllochlorin analogues and their pharmaceutically acceptable salts, and compositions comprising phyllochlorin analogues and their pharmaceutically acceptable salts. Phyllochlorin analogues and pharmaceutically acceptable salts thereof are suitable for use in photodynamic therapy, cytoluminescent therapy and photodynamic diagnosis, for example, for treating or detecting a tumour, or for antiviral treatment. The present invention also relates to the use of phyllochlorin analogues and pharmaceutically acceptable salts thereof in the manufacture of a phototherapeutic or photodiagnostic agent, and to a method of photodynamic therapy, cytoluminescent therapy or photodynamic diagnosis, for example, for treating or detecting a tumour, or for antiviral treatment.

The structure of ‘phyllochlorin’ is shown below:

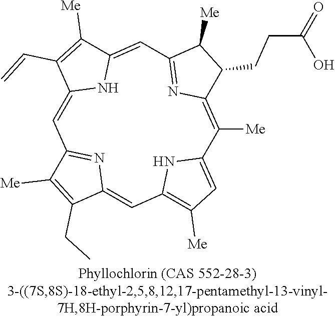 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C]

### BACKGROUND ART

Porphyrins and their analogues are known photosensitive chemical compounds, which can absorb light photons and emit them at higher wavelengths. There are many applications for such unique properties and PDT (photodynamic therapy) is one of them.

Presently, there are two generations of photosensitizers for PDT. The first generation comprises heme porphyrins (blood derivatives), and the second for the most part are chlorophyll analogues. The later compounds are known as chlorins and bacteriochlorins.

Chlorin e4 has been shown to display good photosensitive activity. It was indicated that chlorin e4 has a protective effect against indomethacin-induced gastric lesions in rats and TAA- or CCl4-induced acute liver injuries in mice. It was therefore suggested that chlorin e4 may be a promising new drug candidate for anti-gastrelcosis and liver injury protection. WO 2009/040411 suggests the use of a chlorin e4 zinc complex in photodynamic therapy and WO 2014/091241 suggests the use of chlorin e4 disodium in photodynamic therapy.

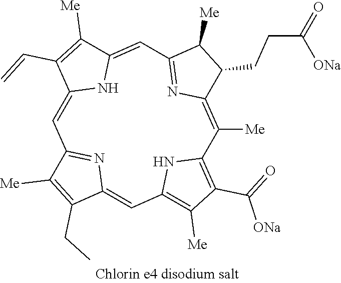

However, there is an ongoing need for better photosensitizers. There is a need for compounds that have a high singlet oxygen quantum yield and for compounds that have a strong photosensitizing ability, preferably in organic and aqueous media. There is also a need for compounds that have a high fluorescence quantum yield. In addition, there is a need for compounds and/or compositions which have a higher phototoxicity, a lower dark toxicity, good stability, and/or are easily purified.

### SUMMARY OF THE INVENTION

A first aspect of the present invention provides a compound of formula (I) or a complex of formula (II):

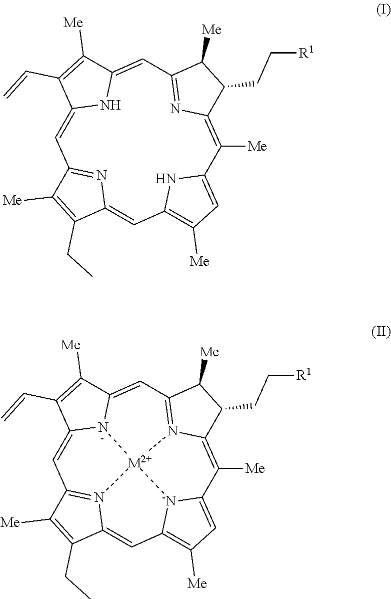

wherein
—R¹ is selected from —C(O)—OR³, —C(O)—SR³, —C(O)—N(R³)₂, —C(S)—OR³, —C(S)—SR³ or —C(S)—N(R³)₂;
—R³, each independently, is selected from —H, —R^{α}—H, —R^{β}, —R^{a}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y or —R^{α}—[R⁶]Y;
—R^{α}—, each independently, is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P or Se in its carbon skeleton;
—R⁵, each independently, is selected from C₁-C₄ alkyl, C₁-C₄ haloalkyl, halo, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
—R⁶ is —[NC₅H₅] optionally substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
n is 1, 2, 3 or 4;
Y is a counter ion;
X is a halo group;
M²⁺ is a metal ion;
   or a pharmaceutically acceptable salt thereof;
   provided that the compound is not:
(1) phyllochlorin free acid; or
(2) phyllochlorin methyl ester.

Phyllochlorin free acid has the structure:

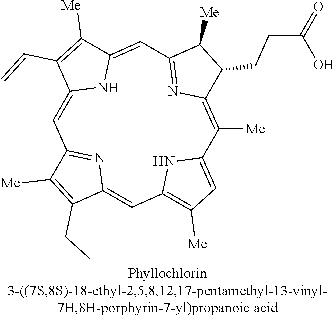 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C]

Phyllochlorin methyl ester has the structure:

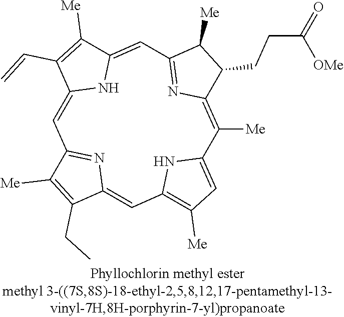 [SMILES: C=Cc1c(C)c2cc3nc(c(C)c4cc(C)c(cc5nc(cc1[nH]2)C(C)=C5CC)[nH]4)[C@@H](CCC(=O)OC)[C@@H]3C]

A second aspect of the present invention provides a compound of formula (I) or a complex of formula (II):

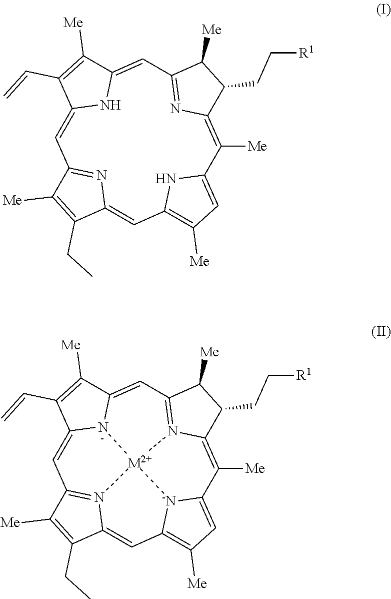

wherein
—R¹ is selected from —C(O)—OR³, —C(O)—SR³, —C(O)—N(R³)₂, —C(S)—OR³, —C(S)—SR³ or —C(S)—N(R³)₂;
—R³, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y or —R^{α}—[R⁶]Y;
—R^{α}—, each independently, is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P or Se in its carbon skeleton;
—R⁵, each independently, is selected from C₁-C₄ alkyl, C₁-C₄ haloalkyl, halo, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
—R⁶ is —[NC₅H₅] optionally substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
n is 1, 2, 3 or 4;
Y is a counter ion;
X is a halo group;
M²⁺ is a metal ion;
   or a pharmaceutically acceptable salt thereof;
   for use in medicine.

In some embodiments of the second aspect of the invention the compound is:
(1) phyllochlorin free acid; or
(2) phyllochlorin methyl ester.

In the context of the present specification, a “hydrocarbyl” substituent group or a hydrocarbyl moiety in a substituent group only includes carbon and hydrogen atoms but, unless stated otherwise, does not include any heteroatoms, such as N, O, S, P or Se in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O, S, P or Se in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₆₀ hydrocarbyl group, more typically a C₁-C₄₀ hydrocarbyl group, more typically a C₁-C₂₀ hydrocarbyl group. More typically a hydrocarbyl group is a C₁-C₁₂ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O, S, P or Se in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more (such as one, two, three or four) heteroatoms, e.g. N, O, S, P or Se in the ring structure. Examples of heterocyclic groups include heteroaryl groups as discussed below and non-aromatic heterocyclic groups such as azetidinyl, azetinyl, tetrahydrofuranyl, pyrrolidinyl, tetrahydrothiophenyl, tetrahydropyranyl, piperidinyl, piperazinyl, morpholinyl, thiomorpholinyl, oxetanyl, thietanyl, pyrazolidinyl, imidazolidinyl, dioxolanyl, oxathiolanyl, thianyl and dioxanyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

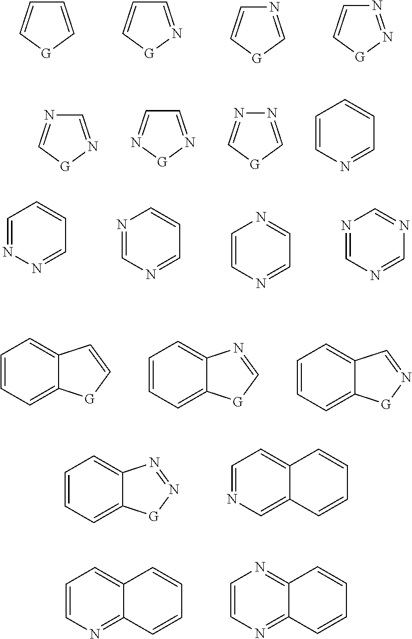

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

For the purposes of the present specification, in an optionally substituted group or moiety (such as —R^{β}):
(i) each hydrogen atom may optionally be replaced by a monovalent substituent independently selected from halo; —CN; —NO₂; —N₃; —R^{x}; —OH; —OR^{x}; —R^{y}-halo; —R^{y}—CN; —R^{y}—NO₂; —R^{y}—N₃; —R^{y}—R^{x}; —R^{y}—OH; —R^{y}—OR^{x}; —SH; —SR^{x}; —SOR^{x}; —SO₂H; —SO₂R^{x}; —SO₂NH₂; —SO₂NHR^{x}; —SO₂N(R^{x})₂; —R^{y}—SH; —R^{y}—SR^{x}; —R^{y}—SOR^{x}; —R^{y}—SO₂H; —R^{y}—SO₂R^{x}; —R^{y}—SO₂NH₂; —R^{y}—SO₂NHR^{x}; —R^{y}—SO₂N(R^{x})₂; —NH₂; —NHR^{x}; —N(R^{x})₂; —N⁺(R^{x})₃; —R^{y}—NH₂; —R^{y}—NHR^{x}; —R^{y}—N(R^{x})₂; —R^{y}—N⁺(R^{x})₃; —CHO; —COR^{x}; —COOH; —COOR^{x}; —OCOR^{x}; —R^{y}—CHO; —R^{y}—COR^{x}; —R^{y}—COOH; —R^{y}—COOR^{x}; or —R^{y}—OCOR^{x}; and/or
(ii) any two hydrogen atoms attached to the same carbon atom may optionally be replaced by a π-bonded substituent independently selected from oxo (═O), ═S, ═NH, or ═NR^{x}; and/or
(iii) any two hydrogen atoms attached to the same or different atoms, within the same optionally substituted group or moiety, may optionally be replaced by a bridging substituent independently selected from —O—, —S—, —NH—, —N(R^{x})—, —N⁺(R^{x})₂— or —R^{y}—;
   wherein each —R^{y}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{x} groups; and
   wherein each —R^{x} is independently selected from a C₁-C₆ alkyl, C₂-C₆ alkenyl, C₂-C₆ alkynyl or C₂-C₆ cyclic group, or wherein any two or three —R^{x} attached to the same nitrogen atom may, together with the nitrogen atom to which they are attached, form a C₂-C₇ cyclic group, and wherein any —R^{x} may optionally be substituted with one or more C₁-C₄ alkyl, C₁-C₄ haloalkyl, —O(C₁-C₄ alkyl), —O(C₁-C₄ haloalkyl), halo, —OH, —NH₂, —CN, or oxo (═O) groups.

Typically a substituted group comprises 1, 2, 3 or 4 substituents, more typically 1, 2 or 3 substituents, more typically 1 or 2 substituents, and more typically 1 substituent.

Unless stated otherwise, any divalent bridging substituent (e.g. —O—, —S—, —NH—, —N(R^{x})—, —N⁺(R^{x})₂— or —R^{y}—) of an optionally substituted group or moiety must only be attached to the specified group or moiety and may not be attached to a second group or moiety, even if the second group or moiety can itself be optionally substituted.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

Unless stated otherwise, any reference to a compound or group is to be considered a reference to all tautomers of that compound or group.

Where reference is made to a hydrocarbyl or other group including one or more heteroatoms N, O, S, P or Se in its carbon skeleton, or where reference is made to a carbon atom of a hydrocarbyl or other group being replaced by an N, O, S, P or Se atom, what is intended is that:

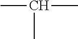

is replaced by

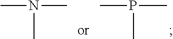

—CH₂— is replaced by —NH—, —PH—, —O—, —S— or —Se—;
—CH₃ is replaced by —NH₂, —PH₂, —OH, —SH or —SeH;
—CH═ is replaced by —N═ or —P═;
CH₂═ is replaced by NH═, PH═, O═, S═ or Se═; or
CH≡ is replaced by N≡ or P≡;
provided that the resultant group comprises at least one carbon atom. For example, methoxy, dimethylamino and aminoethyl groups are considered to be hydrocarbyl groups including one or more heteroatoms N, O, S, P or Se in their carbon skeleton.

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O, S, P or Se are to be counted as carbon atoms when calculating the number of carbon atoms in a Cₓ-C_{y} group. For example, a morpholinyl group is to be considered a C₆ heterocyclic group, not a C₄ heterocyclic group.

The π electrons of the chlorin ring are delocalised and therefore the chlorin ring can be depicted by more than one resonance structure. Resonance structures are different ways of drawing the same compound. Two of the resonance structures of the chlorin ring are depicted directly below:

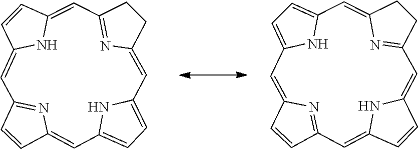 [SMILES: C1=Cc2cc3ccc(cc4nc(cc5ccc(cc1n2)[nH]5)CC4)[nH]3 ; C1=Cc2cc3ccc(cc4nc(cc5ccc(cc1n2)[nH]5)CC4)[nH]3]

Typically a complex comprises a central metal atom or ion known as the coordination centre and a bound molecule or ion which is known as a ligand. In the present specification, the bond between the coordination centre and the ligand is depicted as so shown in the complex on the below left (where the attraction between an anionic ligand and a central metal cation is represented by four dashed lines), but equivalently it could be depicted as shown in the complex on the below right (where the attraction between a ligand molecule and a central metal atom is represented by two covalent bonds and two dashed lines):

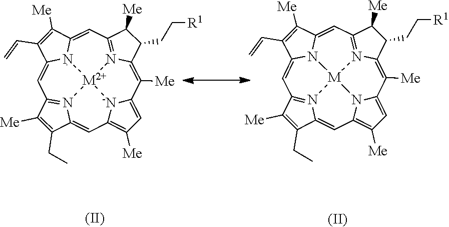

In the context of the present specification, the term “phyllochlorin analogues” encompasses the compounds of the present invention which includes phyllochlorin free acid in the second aspect of the present invention.

As used herein —[NC₅H₅]Y refers to:

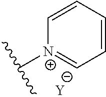

In one embodiment of the first or second aspect of the present invention, Y is a counter ion selected from halides (for example fluoride, chloride, bromide, or iodide) or other inorganic anions (for example nitrate, perchlorate, sulfate, bisulfate, or phosphate) or organic anions (for example propanoate, butyrate, glycolate, lactate, mandelate, citrate, acetate, benzoate, salicylate, succinate, malate, tartrate, fumarate, maleate, hydroxymaleate, galactarate, gluconate, pantothenate, pamoate, methanesulfonate, trifluoromethanesulfonate, ethanesulfonate, 2-hydroxyethanesulfonate, benzenesulfonate, toluene-p-sulfonate, naphthalene-2-sulfonate, camphorsulfonate, ornithinate, glutamate, or aspartate). In one embodiment, Y is fluoride, chloride, bromide or iodide. In one embodiment, Y is chloride or bromide.

In one embodiment of the first or second aspect of the present invention, X is a halo group selected from fluoro, chloro, bromo, or iodo. In one embodiment, X is chloro or bromo.

In one embodiment of the first or second aspect of the present invention, M²⁺ is a metal ion selected from Zn²⁺, Cu²⁺, Fe²⁺, Pd²⁺ or Pt²⁺. In one embodiment, M²⁺ is Zn²⁺.

In one embodiment of the first or second aspect of the present invention, there is provided a compound of formula (I).

—R¹ is selected from —C(O)—OR³, —C(O)—SR³, —C(O)—N(R³)₂, —C(S)—OR³, —C(S)—SR³ or —C(S)—N(R³)₂. In one embodiment, —R¹ is selected from —C(O)—OR³, —C(O)—SR³, —C(O)—N(R³)₂ or —C(S)—N(R³)₂. In one embodiment, —R¹ is selected from —C(O)—OR³, —C(O)—SR³ or —C(O)—N(R³)₂.

In one embodiment of the first or second aspect of the present invention, —R¹ is selected from —C(O)—OR³, —C(O)—SR³, —C(O)—N(R³)₂, —C(S)—OR³, —C(S)—SR³ or —C(S)—N(R³)₂, and each —R³ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R¹ is selected from —C(O)—OR³, —C(O)—SR³ or —C(O)—N(R³)₂, and each —R³ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R¹ is selected from —C(O)—OR³ or —C(O)—SR³, and —R³ is selected from —R^{α}—OR^{β} or —R^{α}—SR^{β}, and —R^{β} is a saccharidyl group. Typically in these embodiments, —R^{α}— is a C₁-C₁₂ alkylene group (preferably a C₁-C₈ alkylene group, or a C₁-C₆ alkylene group), a —(CH₂CH₂O)ₘ— group or a —(CH₂CH₂S)ₘ— group, all optionally substituted, wherein m is 1, 2, 3 or 4.

In one embodiment of the first or second aspect of the present invention, —R¹ is selected from —C(O)—OR³, —C(O)—SR³, —C(O)—N(R³)(R^{3′}) or —C(S)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—OR^{β} or —R^{α}—SR^{β}, and —R^{β} is a saccharidyl group, and —R^{3′} is H or C₁-C₄ alkyl (preferably methyl). Typically in these embodiments, —R^{α}— is a C₁-C₁₂ alkylene group (preferably a C₁-C₈ alkylene group, or a C₁-C₆ alkylene group), a —(CH₂CH₂O)ₘ— group or a —(CH₂CH₂S)ₘ— group, all optionally substituted, wherein m is 1, 2, 3 or 4.

An —R^{3′} group refers to an —R³ group attached to the same atom as another —R³ group. —R³ and —R^{3′} may be the same or different. Preferably —R³ and —R^{3′} are different.

In one embodiment of the first or second aspect of the present invention, —R¹ is selected from —C(O)—OR³, —C(O)—SR³, —C(O)—N(R³)(R^{3′}) or —C(S)—N(R³)(R^{3′}), wherein —R³ is selected from —R^{α}—R^{β} or —R^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—R^{β} or —R^{β}, and —R^{β} is a saccharidyl group, and —R^{3′} is H or C₁-C₄ alkyl (preferably methyl). Typically in these embodiments, —R^{α}— is a C₁-C₁₂ alkylene group (preferably a C₁-C₈ alkylene group, or a C₁-C₆ alkylene group), a —(CH₂CH₂O)ₘ— group or a —(CH₂CH₂S)ₘ— group, all optionally substituted, wherein m is 1, 2, 3 or 4.

In any of the embodiments in the four preceding paragraphs, the saccharidyl group may optionally be substituted, for example, with a protecting group such as acetyl or a natural amino acid such as valine. Amino acids can be attached to saccharidyl groups, for example, by forming an ester between a carboxylic acid group of the amino acid and a hydroxyl group of the saccharidyl group.

In one embodiment of the first or second aspect of the present invention, —R¹ is selected from —C(O)—OR³, —C(O)—SR³, —C(O)—N(R³)(R^{3′}) or —C(S)—N(R³)(R^{3′}), wherein —R³ is selected from —R^{α}—R^{β} or —R^{β}, and —R^{β} is a C₁-C₈ alkylene group optionally substituted with one or more (such as one, two, three, four, five, six, seven or eight) hydroxyl groups, and —R^{3′} is H or C₁-C₄ alkyl (preferably methyl). In one embodiment, —R¹ is —C(O)—N(R³)(R^{3′}), wherein —R³ is selected from —R^{α}—R^{β} or —R^{β}, and —R^{β} is a C₁-C₈ alkylene group optionally substituted with one or more (such as one, two, three, four, five, six, seven or eight) hydroxyl groups, and —R^{3′} is H or C₁-C₄ alkyl (preferably methyl). Typically in these embodiments, —R^{α}— is an unsubstituted C₁-C₆ alkylene group, or an unsubstituted C₁-C₄ alkylene group, or an unsubstituted C₁-C₂ alkylene group.

In one embodiment of the first or second aspect of the present invention, —R¹ is selected from —C(O)—OR³, —C(O)—SR³, —C(O)—N(R³)(R^{3′}) or —C(S)—N(R³)(R^{3′}); wherein —R³ is selected from —R^{α}—H or —R^{α}—OH; —R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and —R^{3′} is H or C₁-C₄ alkyl (preferably methyl). In one embodiment, —R¹ is —C(O)—N(R³)(R^{3′}); wherein —R³ is selected from —R^{α}—H or —R^{α}—OH; —R^{α}— is selected from a C₁-C₁₂ alkylene group, wherein one or more carbon atoms in the backbone of the alkylene group may optionally be replaced by one or more heteroatoms O or S; and —R^{3′} is H or C₁-C₄ alkyl (preferably methyl).

In one embodiment of the first or second aspect of the present invention, —R¹ is selected from —C(O)—OR³, —C(O)—SR³, —C(O)—N(R³)(R^{3′}) or —C(S)—N(R³)(R^{3′}); wherein —R³ is —R^{β}; —R^{β} is a C₁-C₁₂ alkyl group optionally substituted with one or more (such as one, two, three, four or five) substituents independently selected from halo, —CN, —NO₂, —N₃, —OH, —OR^{x}, —SH, —SR^{x}, —SOR^{x}, —SO₂H, —SO₂R^{x}, —SO₂NH₂, —SO₂NHR^{x}, —SO₂N(R^{x})₂, —NH₂, —NHR^{x}, —N(R^{x})₂, —N⁺(R^{x})₃, —CHO, —COR^{x}, —COOH, —COOR^{x}, —OCOR^{x}, or —NH—CO—CR^{z}—NH₂; each —R^{x} is independently selected from C₁-C₄ alkyl; —R^{z} is the side chain of a natural amino acid; and —R^{3′} is H or C₁-C₄ alkyl (preferably methyl). In one embodiment, —R¹ is —C(O)—N(R³)(R^{3′}); wherein —R³ is —R^{β}; —R^{β} is a C₁-C₈ alkyl group optionally substituted with one or more (such as one, two or three) substituents independently selected from halo, —CN, —NO₂, —N₃, —OH, —OR^{x}, —SH, —SR^{x}, —SOR^{x}, —SO₂H, —SO₂R^{x}, —SO₂NH₂, —SO₂NHR^{x}, —SO₂N(R^{x})₂, —NH₂, —NHR^{x}, —N(R^{x})₂, —N⁺(R^{x})₃, —CHO, —COR^{x}, —COOH, —COOR^{x}, —OCOR^{x}, or —NH—CO—CR^{z}—NH₂; each —R^{x} is independently selected from C₁-C₄ alkyl; —R^{z} is the side chain of a natural amino acid; and —R^{3′} is H or C₁-C₄ alkyl (preferably methyl).

In one embodiment of the first or second aspect of the present invention, —R¹ is selected from —C(O)—OR³, —C(O)—SR³, —C(O)—N(R³)(R^{3′}) or —C(S)—N(R³)(R^{3′}); wherein —R³ is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups; n is 1, 2, 3 or 4; Y is fluoride, chloride, bromide or iodide; and —R^{3′} is H or C₁-C₄ alkyl (preferably methyl). In one embodiment, —R¹ is —C(O)—N(R³)(R^{3′}); wherein —R³ is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups; n is 1, 2, 3 or 4; Y is fluoride, chloride, bromide or iodide; and —R^{3′} is H or C₁-C₄ alkyl (preferably methyl). Typically in these embodiments, —R^{α}— is a C₁-C₁₂ alkylene group (preferably a C₁-C₈ alkylene group, or a C₁-C₆ alkylene group), a —(CH₂CH₂O)ₘ group or a —(CH₂CH₂S)ₘ— group, all optionally substituted, wherein m is 1, 2, 3 or 4.

In one embodiment of the first or second aspect of the present invention, —R¹ is —C(O)—OR³, wherein —R³ is selected from C₁-C₄ alkyl (preferably methyl) or a cation (such as a lithium, sodium, potassium, magnesium, calcium, ammonium, amine (such as choline or meglumine), or amino acid (such as arginine) cation).

In one embodiment of the first or second aspect of the present invention, —R¹ is —C(O)—N(R³)₂. In one embodiment, —R¹ is —C(O)—N(C₁-C₄ alkyl)(R³) or —C(O)—NHR³. In one embodiment, —R¹ is —C(O)—N(CH₃)(R³) or —C(O)—NHR³. In one embodiment, —R¹ is —C(O)—N(C₁-C₄ alkyl)(R³). In one embodiment, —R¹ is —C(O)—N(CH₃)(R³).

In one embodiment of the first or second aspect of the present invention, each —R^{α}— is independently a C₁-C₁₂ alkylene group, a —(CH₂CH₂O)ₘ— group or a —(CH₂CH₂S)ₘ— group, all optionally substituted, wherein m is 1, 2, 3 or 4. In one embodiment, each —R^{α}— is independently a C₁-C₁₂ alkylene group or a —(CH₂CH₂O)ₘ— group, both optionally substituted, wherein m is 1, 2, 3 or 4. In one embodiment, each —R^{α}— is independently an optionally substituted —(CH₂CH₂O)ₘ— group, wherein m is 1, 2, 3 or 4.

In one embodiment of the first or second aspect of the present invention, each —R^{α}— is independently a C₁-C₈ alkylene group, or a C₁-C₆ alkylene group, or a C₂-C₄ alkylene group, all optionally substituted.

In one embodiment of the first or second aspect of the present invention, each —R^{α}— is independently unsubstituted or substituted with one or more substituents independently selected from halo, C₁-C₄ alkyl, or C₁-C₄ haloalkyl. In one embodiment, each —R^{α}— is independently unsubstituted or substituted with one or two substituents so independently selected from halo, C₁-C₄ alkyl, or C₁-C₄ haloalkyl. In one embodiment, each —R^{α}— is unsubstituted.

In one embodiment of the first or second aspect of the present invention, each —R^{β} is independently a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In one embodiment of the first or second aspect of the present invention, at least one —R^{β} is independently a C₁-C₆ alkyl group, or a C₁-C₄ alkyl group, or a methyl group, all optionally substituted. In one embodiment, each —R^{β} is independently a C₁-C₆ alkyl group, or a C₁-C₄ alkyl group, or a methyl group, all optionally substituted.

In one embodiment of the first or second aspect of the present invention, at least one —R^{β} is independently a saccharidyl group. In one embodiment, each —R^{β} is independently a saccharidyl group.

In one embodiment of the first or second aspect of the present invention, each —R^{β} is independently unsubstituted or substituted with one or more substituents independently selected from halo, C₁-C₄ alkyl, or C₁-C₄ haloalkyl. In one embodiment, each —R^{β} is independently unsubstituted or substituted with one or two substituents independently selected from halo, C₁-C₄ alkyl, or C₁-C₄ haloalkyl. In one embodiment, each —R^{β} is unsubstituted.

In one embodiment of the first or second aspect of the present invention, each —R³ is independently selected from —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]y, —R^{α}—[P(R⁵)₃]Y, or —R^{α}—[NC₅H₅]Y. In one embodiment, each —R³ is independently selected from —R^{α}—OR^{β}, —R^{α}—SR^{β}, —R^{α}—S(O)R^{β} or —R^{α}—S(O)₂R^{β}. In one embodiment, each —R³ is independently selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, each —R³ is independently selected from —R^{α}—OR^{β} or —R^{α}—SR^{β}. In one embodiment, each —R³ is independently selected from —R^{α}—OR^{β} or —R^{α}—SR^{β}, and —R^{β} is a saccharidyl group.

In one embodiment of the first or second aspect of the present invention, at least one —R³ is independently selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, at least one —R³ is independently selected from —R^{α}—OR^{β} or —R^{α}—SR^{β}, and —R^{β} is a saccharidyl group.

For the purposes of the present invention, a “saccharidyl group” is any group comprising at least one monosaccharide subunit, wherein each monosaccharide subunit may optionally be substituted and/or modified. Typically, a saccharidyl group consist of one or more monosaccharide subunits, wherein each monosaccharide subunit may optionally be substituted and/or modified.

Typically, a carbon atom of a single monosaccharide subunit of each saccharidyl group is directly attached to the remainder of the compound, most typically via a single bond.

For the purposes of the present specification, where it is stated that a first atom or group is “directly attached” to a second atom or group it is to be understood that the first atom or group is covalently bonded to the second atom or group with no intervening atom(s) or group(s) being present. For example, for the group —(C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

Typically, each saccharidyl group is derived from the corresponding saccharide by substitution of a hydroxyl group of the saccharide with the group defined by the remainder of the compound.

A single bond between an anomeric carbon of a monosaccharide subunit and a substituent is called a glycosidic bond. A glycosidic group is linked to the anomeric carbon of a monosaccharide subunit by a glycosidic bond. The bond between the saccharidyl group and the remainder of the compound may be a glycosidic or a non-glycosidic bond. Typically, the bond between the saccharidyl group and the remainder of the compound is a glycosidic bond, such that the saccharidyl group is a glycosyl group. Where the bond between the saccharidyl group and the remainder of the compound is a glycosidic bond, the glycosidic bond may be in the α or β configuration. Typically, such a glycosidic bond is in the β configuration.

For the purposes of the present invention, where a saccharidyl group “contains x monosaccharide subunits”, this means that the saccharidyl group has x monosaccharide subunits and no more. In contrast, where a saccharidyl group “comprises x monosaccharide subunits”, this means that the saccharidyl group has x or more monosaccharide subunits.

Each saccharidyl group may be independently selected from a monosaccharidyl, disaccharidyl, oligosaccharidyl or polysaccharidyl group. As will be understood, a monosaccharidyl group contains a single monosaccharide subunit. Similarly, a disaccharidyl group contains two monosaccharide subunits. As used herein, an “oligosaccharidyl group” contains from 2 to 9 monosaccharide subunits. Examples of oligosaccharidyl groups include trisaccharidyl, tetrasaccharidyl, pentasaccharidyl, hexasaccharidyl, heptasaccharidyl, octasaccharidyl and nonasaccharidyl groups. As used herein, a “polysaccharidyl group” contains 10 or more monosaccharide subunits (such as 10-50, or 10-30, or 10-20, or 10-15 monosaccharide subunits).

Each monosaccharide subunit within a disaccharidyl, oligosaccharidyl or polysaccharidyl group may be the same or different. Each monosaccharide subunit within a disaccharidyl, oligosaccharidyl or polysaccharidyl group may be connected to another monosaccharide subunit within the group via a glycosidic or a non-glycosidic bond. Typically each monosaccharide subunit within a disaccharidyl, oligosaccharidyl or polysaccharidyl group is connected to another monosaccharide subunit within the group via a glycosidic bond, which may be in the α or β configuration.

Each oligosaccharidyl or polysaccharidyl group may be a linear, branched or macrocyclic oligosaccharidyl or polysaccharidyl group. Typically, each oligosaccharidyl or polysaccharidyl group is a linear or branched oligosaccharidyl or polysaccharidyl group.

In one embodiment, at least one —R^{β} is a monosaccharidyl or disaccharidyl group.

In a further embodiment, at least one —R^{β} is a monosaccharidyl group. For example, at least one —R^{β} may be a glycosyl group containing a single monosaccharide subunit, so wherein the monosaccharide subunit may optionally be substituted and/or modified. Typically at least one —R^{β} is a glycosyl group containing a single monosaccharide subunit, wherein the monosaccharide subunit may optionally be substituted. More typically, at least one —R^{β} is a glycosyl group containing a single monosaccharide subunit, wherein the monosaccharide subunit is unsubstituted.

In one embodiment, at least one —R^{β} is an aldosyl group, wherein the aldosyl group may optionally be substituted and/or modified. For example, at least one —R^{β} may be selected from a glycerosyl, aldotetrosyl (such as erythrosyl or threosyl), aldopentosyl (such as ribosyl, arabinosyl, xylosyl or lyxosyl) or aldohexosyl (such as allosyl, altrosyl, glucosyl, mannosyl, gulosyl, idosyl, galactosyl or talosyl) group, any of which may optionally be substituted and/or modified.

In another embodiment, at least one —R^{β} is a ketosyl group, wherein the ketosyl group may optionally be substituted and/or modified. For example, at least one —R^{β} may be selected from an erythrulosyl, ketopentosyl (such as ribulosyl or xylulosyl) or ketohexosyl (such as psicosyl, fructosyl, sorbosyl or tagatosyl) group, any of which may optionally be substituted and/or modified.

Each monosaccharide subunit may be present in a ring-closed (cyclic) or open-chain (acyclic) form. Typically, each monosaccharide subunit in at least one —R^{β} is present in a ring-closed (cyclic) form. For example, at least one —R^{β} may be a glycosyl group containing a single ring-closed monosaccharide subunit, wherein the monosaccharide subunit may optionally be substituted and/or modified. Typically in such a scenario, at least one —R^{β} is a pyranosyl or furanosyl group, such as an aldopyranosyl, aldofuranosyl, ketopyranosyl or ketofuranosyl group, any of which may optionally be substituted and/or modified. More typically, at least one —R^{β} is a pyranosyl group, such as an aldopyranosyl or ketopyranosyl group, any of which may optionally be substituted and/or modified.

In one embodiment, at least one —R^{β} is selected from a ribopyranosyl, arabinopyranosyl, xylopyranosyl, lyxopyranosyl, allopyranosyl, altropyranosyl, glucopyranosyl, mannopyranosyl, gulopyranosyl, idopyranosyl, galactopyranosyl or talopyranosyl group, any of which may optionally be substituted and/or modified.

In a further embodiment, at least one —R^{β} is a glucosyl group, such as a glucopyranosyl group, wherein the glucosyl or the glucopyranosyl group may optionally be substituted and/or modified. Typically, at least one —R^{β} is a glucosyl group, wherein the glucosyl group is optionally substituted. More typically, at least one —R^{β} is an unsubstituted glucosyl group.

Each monosaccharide subunit may be present in the D- or L-configuration. Typically, each monosaccharide subunit is present in the configuration in which it most commonly occurs in nature.

In one embodiment, at least one —R^{β} is a D-glucosyl group, such as a D-glucopyranosyl group, wherein the D-glucosyl or the D-glucopyranosyl group may optionally be substituted and/or modified. Typically, at least one —R^{β} is a D-glucosyl group, wherein the D-glucosyl group is optionally substituted. More typically, at least one —R^{β} is an unsubstituted D-glucosyl group.

For the purposes of the present invention, in a substituted monosaccharidyl group or monosaccharide subunit:
(a) one or more of the hydroxyl groups of the monosaccharidyl group or monosaccharide subunit are each independently replaced with —H, —F, —Cl, —Br, —I, —CF₃, —CCl₃, —CBr₃, —CI₃, —SH, —NH₂, —N₃, —NH═NH₂, —CN, —NO₂, —COOH, —R^{b}, —O—R^{b}, —S—R^{b}, —R^{α}—O—R^{b}, —R^{α}—S—R^{b}, —SO—R^{b}, —SO₂—R^{b}, —SO₂—OR^{b}, —O—SO—R^{b}, —O—SO₂—R^{b}, —O—SO₂—OR^{b}, —NR^{b}—SO—R^{b}, —NR^{b}—SO₂—R^{b}, —NR^{b}—SO₂—OR^{b}, —R^{a}—SO—R^{b}, —R^{a}—SO₂—R^{b}, —R^{a}—SO₂—OR^{b}, —SO—N(R^{b})₂, —SO₂—N(R^{b})₂, —O—SO—N(R^{b})₂, —O—SO₂—N(R^{b})₂, —NR^{b}—SO—N(R^{b})₂, —NR^{b}—SO₂—N(R^{b})₂, —R^{a}—SO—N(R^{b})₂, —R^{a}—SO₂—N(R^{b})₂, —N(R^{b})₂, —N(R^{b})₃⁺, —R^{a}—N(R^{b})₂, —R^{a}—N(R^{b})₃⁺, —P(R^{b})₂, —PO(R^{b})₂, —OP(R^{b})₂, —OPO(R^{b})₂, —R^{a}—P(R^{b})₂, —R^{a}—PO(R^{b})₂, —OSi(R^{b})₃, —R^{a}—Si(R^{b})₃, —CO—R^{b}, —CO—OR^{b}, —CO—N(R^{b})₂, —O—CO—R^{b}, —O—CO—OR^{b}, —O—CO—N(R^{b})₂, —NR^{b}—CO—R^{b}, —NR^{b}—CO—OR^{b}, —NR^{b}—CO—N(R^{b})₂, —R^{a}—CO—R^{b}, —R^{a}—CO—OR^{b}, or —R^{a}—CO—N(R^{b})₂; and/or
(b) one, two or three hydrogen atoms directly attached to a carbon atom of the monosaccharidyl group or monosaccharide subunit are each independently replaced with —F, —Cl, —Br, —I, —CF₃, —CCl₃, —CBr₃, —CI₃, —OH, —SH, —NH₂, —N₃, —NH═NH₂, —CN, —NO₂, —COOH, —R^{b}, —O—R^{b}, —S—R^{b}, —R^{α}—O—R^{b}, —R^{α}—S—R^{b}, —SO—R^{b}, —SO₂—R^{b}, —SO₂—OR^{b}, —O—SO—R^{b}, —O—SO₂—R^{b}, —O—SO₂—OR^{b}, —NR^{b}—SO—R^{b}, —NR^{b}—SO₂—R^{b}, —NR^{b}—SO₂—OR^{b}, —R^{a}—SO—R^{b}, —R^{a}—SO₂—R^{b}, —R^{a}—SO₂—OR^{b}, —SO—N(R^{b})₂, —SO₂—N(R^{b})₂, —O—SO—N(R^{b})₂, —O—SO₂—N(R^{b})₂, —NR^{b}—SO—N(R^{b})₂, —NR^{b}—SO₂—N(R^{b})₂, —R^{a}—SO—N(R^{b})₂, —R^{a}—SO₂—N(R^{b})₂, —N(R^{b})₂, —N(R^{b})₃⁺, —R^{a}—N(R^{b})₂, —R^{a}—N(R^{b})₃⁺, —P(R^{b})₂, —PO(R^{b})₂, —OP(R^{b})₂, —OPO(R^{b})₂, —R^{a}—P(R^{b})₂, —R^{a}—PO(R^{b})₂, —OSi(R^{b})₃, —R^{a}—Si(R^{b})₃, —CO—R^{b}, —CO—OR^{b}, —CO—N(R^{b})₂, —O—CO—R^{b}, —O—CO—OR^{b}, —O—CO—N(R^{b})₂, —NR^{b}—CO—R^{b}, —NR^{b}—CO—OR^{b}, —NR^{b}—CO—N(R^{b})₂, —R^{a}—CO—R^{b}, —R^{a}—CO—OR^{b}, or —R^{α}—CO—N(R^{b})₂; and/or
(c) one or more of the hydroxyl groups of the monosaccharidyl group or monosaccharide subunit, together with the hydrogen attached to the same carbon atom as the hydroxyl group, are each independently replaced with ═O, ═S, ═NR^{b}, or ═N(R^{b})₂⁺; and/or
(d) any two hydroxyl groups of the monosaccharidyl group or monosaccharide subunit are together replaced with —O—R^{c}—, —S—R^{c}—, —SO—R^{c}—, —SO₂—R^{c}—, or —NR^{b}—R^{c}—; wherein:
   each —R^{a}— is independently a substituted or unsubstituted alkylene, alkenylene or alkynylene group which optionally includes one or more heteroatoms each independently selected from O, N and S in its carbon skeleton and preferably comprises 1-10 carbon atoms;
   each —R^{b} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one or more heteroatoms each independently selected from O, N and S in its carbon skeleton and preferably comprises 1-15 carbon atoms; and
   each —R^{c}— is independently a chemical bond, or a substituted or unsubstituted alkylene, alkenylene or alkynylene group which optionally includes one or more heteroatoms each independently selected from O, N and S in its carbon skeleton and preferably comprises 1-10 carbon atoms;
   provided that the monosaccharidyl group or monosaccharide subunit comprises at least one, preferably at least two or at least three, —OH, —O—R^{b}, —O—SO—R^{b}, —O—SO₂—R^{b}, —O—SO₂—OR^{b}, —O—SO—N(R^{b})₂, —O—SO₂—N(R^{b})₂, —OP(R^{b})₂, —OPO(R^{b})₂, —OSi(R^{b})₃, —O—CO—R^{b}, —O—CO—OR^{b}, —O—CO—N(R^{b})₂, or —O—R^{c}—.

Typically, in a substituted monosaccharidyl group or monosaccharide subunit:
(a) one or more of the hydroxyl groups of the monosaccharidyl group or monosaccharide subunit are each independently replaced with —H, —F, —CF₃, —SH, —NH₂, —N₃, —CN, —NO₂, —COOH, —R^{b}, —O—R^{b}, —S—R^{b}, —N(R^{b})₂, —OPO(R^{b})₂, —OSi(R^{b})₃, —O—CO—R^{b}, —O—CO—OR^{b}, —O—CO—N(R^{b})₂, —NR^{b}—CO—R^{b}, —NR^{b}—CO—OR^{b}, or —NR^{b}—CO—N(R^{b})₂; and/or
(b) one or two of the hydrogen atoms directly attached to a carbon atom of the monosaccharidyl group or monosaccharide subunit are each independently replaced with —F, —CF₃, —OH, —SH, —NH₂, —N₃, —CN, —NO₂, —COOH, —R^{b}, —O—R^{b}, —S—R^{b}, —N(R^{b})₂, —OPO(R^{b})₂, —OSi(R^{b})₃, —O—CO—R^{b}, —O—CO—OR^{b}, —O—CO—N(R^{b})₂, —NR^{b}—CO—R^{b}, —NR^{b}—CO—OR^{b}, or —NR^{b}—CO—N(R^{b})₂; and/or
(c) one hydroxyl group of the monosaccharidyl group or monosaccharide subunit, together with the hydrogen attached to the same carbon atom as the hydroxyl group, is replaced with ═O; and/or
(d) any two hydroxyl groups of the monosaccharidyl group or monosaccharide subunit are together replaced with —O—R^{c}— or —NR^{b}—R^{c}—;
   wherein:
each —R^{b} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one, two or three heteroatoms each independently selected from O and N in its carbon skeleton and comprises 1-8 carbon atoms; and
each —R^{c}— is independently a substituted or unsubstituted alkylene, alkenylene or alkynylene group which optionally includes one, two or three heteroatoms each independently selected from O and N in its carbon skeleton and comprises 1-8 carbon atoms;
provided that the monosaccharidyl group or monosaccharide subunit comprises at least two, preferably at least three, —OH, —O—R^{b}, —OPO(R^{b})₂, —OSi(R^{b})₃, —O—CO—R^{b}, —O—CO—OR^{b}, —O—CO—N(R^{b})₂, or —O—R^{c}—.

In one embodiment, —R^{β} is a saccharidyl group and one or more of the hydroxyl groups of the saccharidyl group are each independently replaced with —O—CO—R^{b}, wherein each —R^{b} is independently C₁-C₄ alkyl, preferably methyl. In one embodiment, —R^{β} is a saccharidyl group and all of the hydroxyl groups of the saccharidyl group are each independently replaced with —O—CO—R^{b}, wherein each —R^{b} is independently C₁-C₄ alkyl, preferably methyl.

In a modified monosaccharidyl group or monosaccharide subunit:
(a) the ring of the modified monosaccharidyl group or monosaccharide subunit, or what would be the ring in the ring-closed form of the modified monosaccharidyl group or monosaccharide subunit, is partially unsaturated; and/or
(b) the ring oxygen of the modified monosaccharidyl group or monosaccharide subunit, or what would be the ring oxygen in the ring-closed form of the modified monosaccharidyl group or monosaccharide subunit, is replaced with —S— or —NR^{d}—, wherein —R^{d} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one or more heteroatoms each independently selected from O, N and S in its carbon skeleton and preferably comprises 1-15 carbon atoms.

Alternately, where the modified monosaccharide subunit forms part of a disaccharidyl, oligosaccharidyl or polysaccharidyl group, —R^{d} may be a further monosaccharide subunit or subunits forming part of the disaccharidyl, oligosaccharidyl or polysaccharidyl group, wherein any such further monosaccharide subunit or subunits may optionally be substituted and/or modified.

Typically, in a modified monosaccharidyl group or monosaccharide subunit:
(a) the ring of the modified monosaccharidyl group or monosaccharide subunit, or what would be the ring in the ring-closed form of the modified monosaccharidyl group or monosaccharide subunit, contains a single C═C; and/or
(b) the ring oxygen of the modified monosaccharidyl group or monosaccharide subunit, or what would be the ring oxygen in the ring-closed form of the modified monosaccharidyl group or monosaccharide subunit, is replaced with —NR^{d}—, wherein —R^{d} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one, two or three heteroatoms each independently selected from O and N in its carbon skeleton and comprises 1-8 carbon atoms.

Typical examples of substituted and/or modified monosaccharide subunits include those corresponding to:
(i) deoxy sugars, such as deoxyribose, fucose, fuculose and rhamnose, wherein a hydroxyl group of the monosaccharidyl group or monosaccharide subunit has been replaced by —H;
(ii) amino sugars, such as glucosamine and galactosamine, wherein a hydroxyl group of the monosaccharidyl group or monosaccharide subunit has been replaced by —NH₂, most typically at the 2-position; and
(iii) sugar acids, containing a —COOH group, such as aldonic acids (e.g. gluconic acid), ulosonic acids, uronic acids (e.g. glucuronic acid) and aldaric acids (e.g. gularic or galactaric acid).

In one embodiment of the first or second aspect of the present invention, at least one —R^{β} is a monosaccharidyl group selected from:

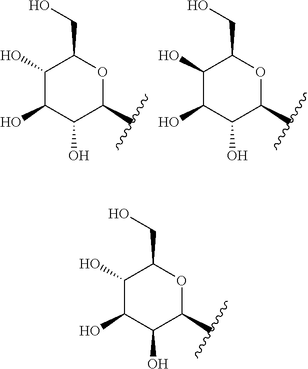

Preferably in the compound or complex according to the first or second aspect of the present invention, at least one —R^{β} is:

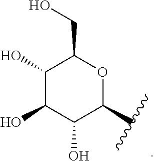 [SMILES: *[C@@H]1O[C@H](CO)[C@@H](O)[C@H](O)[C@H]1O]

In one embodiment of the first or second aspect of the present invention, at least one —R³ is independently selected from —R^{α}—OR^{β}, —R^{α}—SR^{β}, —R^{α}—S(O)R^{β} or —R^{α}—S(O)₂R (preferably from —R^{α}—OR^{β} or —R^{α}—SR^{β}), and —R^{β} is selected from:

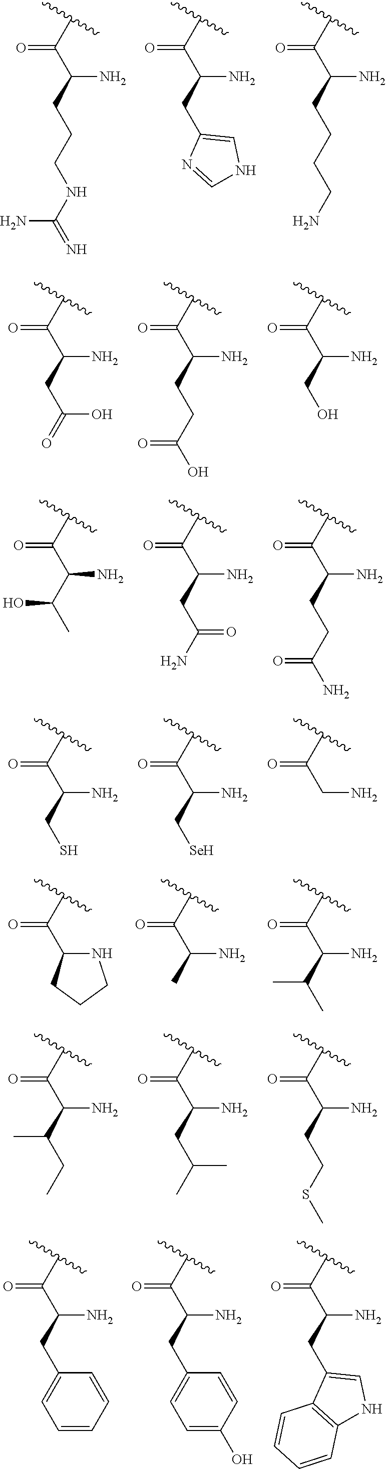

In one embodiment of the first or second aspect of the present invention, at least one —R³ is independently selected from —R^{α}—[N(R⁵)₃]Y, —R^{α}—[P(R⁵)₃]Y, or —R^{α}—[R⁶]Y. In one embodiment, at least one —R³ is independently selected from:

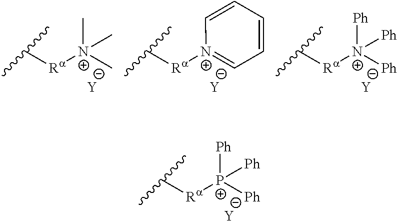

In one embodiment of the first or second aspect of the present invention, each —R⁵ is independently unsubstituted or substituted with one or two substituents. In one embodiment, each —R⁵ is unsubstituted.

In one embodiment of the first or second aspect of the present invention, —R⁶ is unsubstituted or substituted with one or two substituents. In one embodiment, —R⁶ is unsubstituted.

In one embodiment, —R⁶ is not substituted at the 4-position of the pyridine ring with a halo group. In one embodiment, —R⁶ is unsubstituted at the 4-position of the pyridine ring. In one embodiment, —R⁶ is unsubstituted.

Preferably in the compound or complex according to the first or second aspect of the present invention, the compound or complex is:

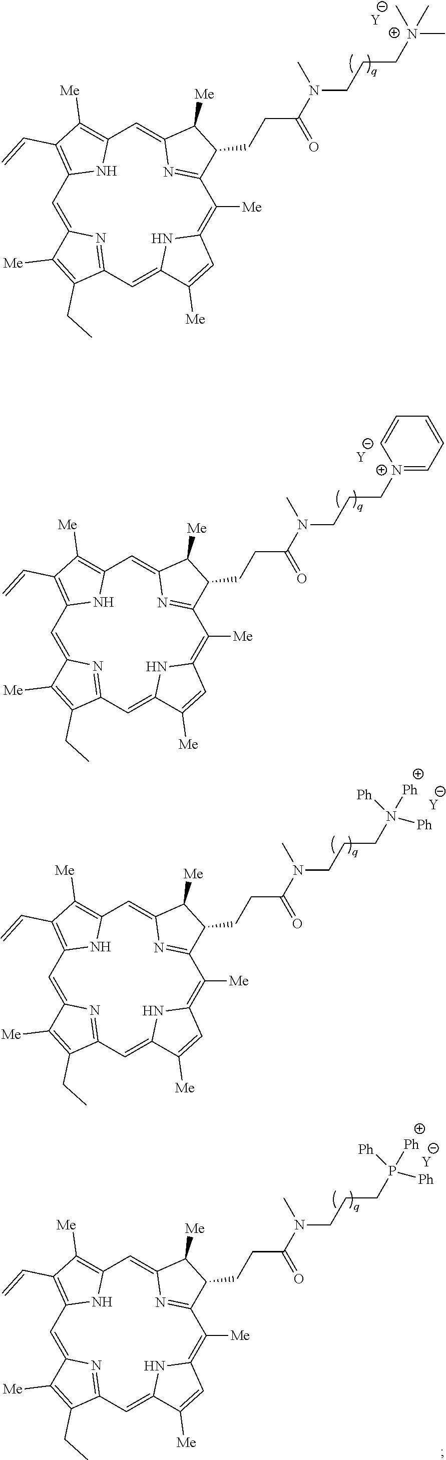

wherein Y is a counter ion, and q is 0, 1, 2, 3 or 4 (preferably q is 1);
or a complex or a pharmaceutically acceptable salt thereof.

Preferably in the compound or complex according to the first or second aspect of the present invention, the compound or complex is:

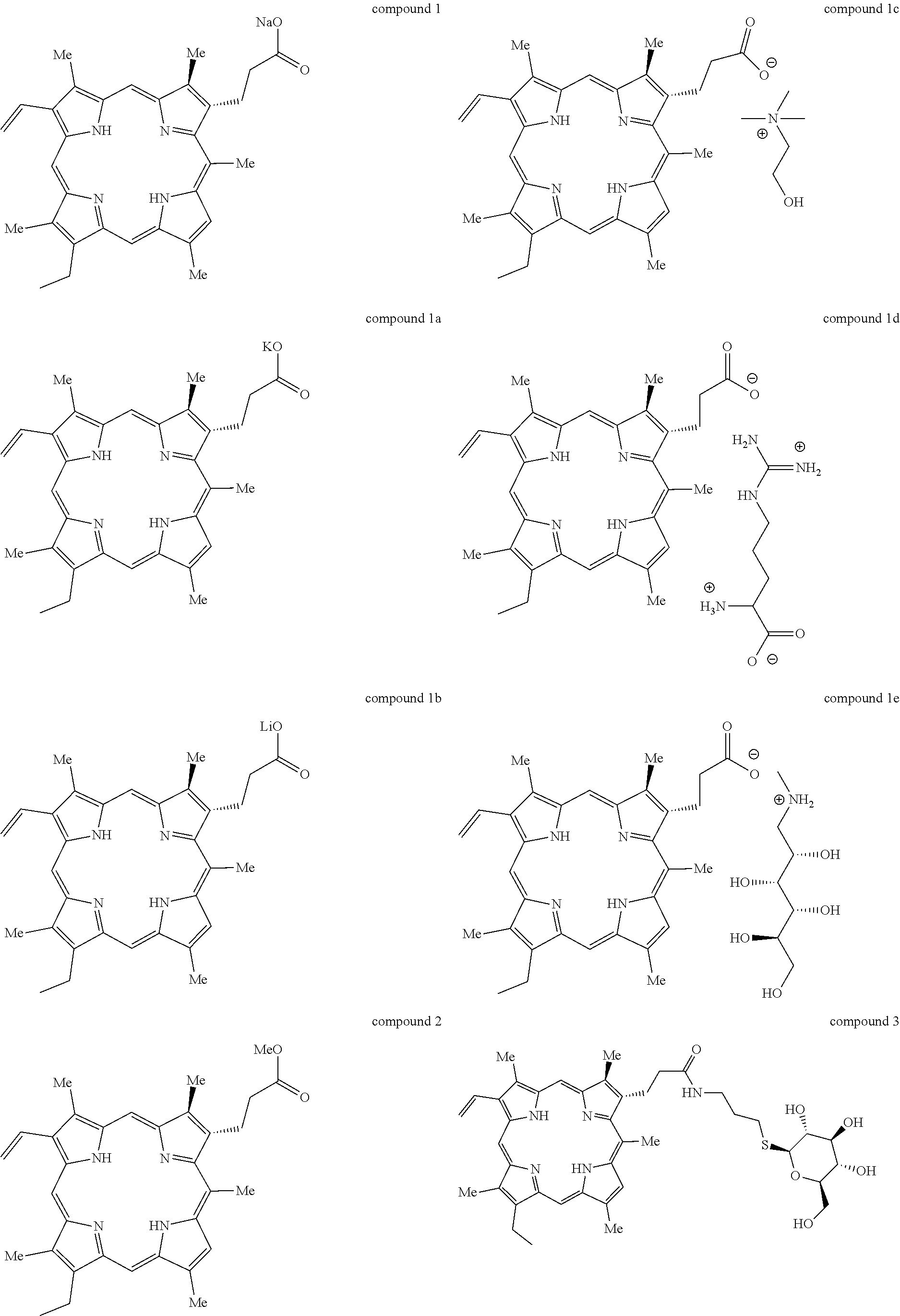

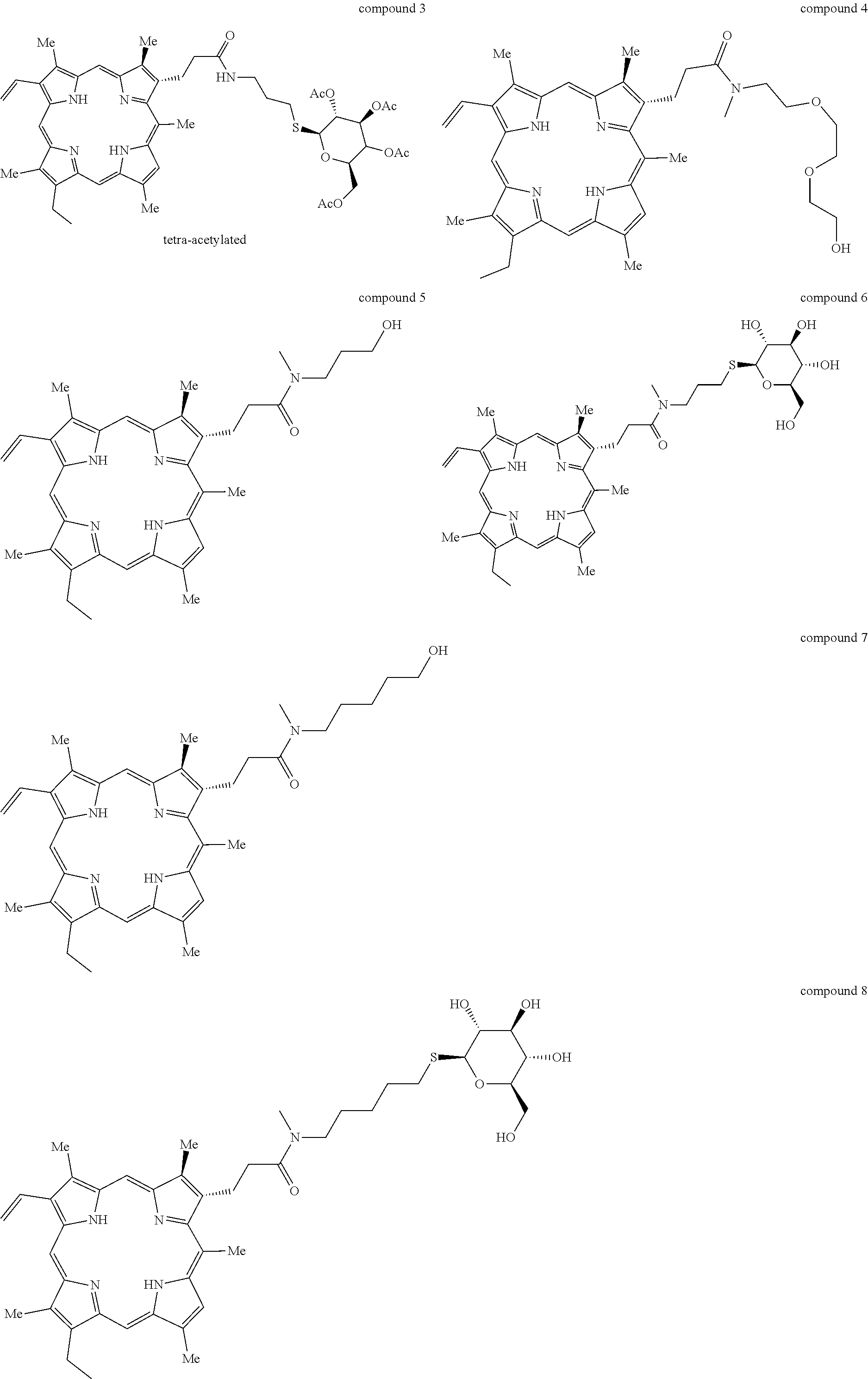

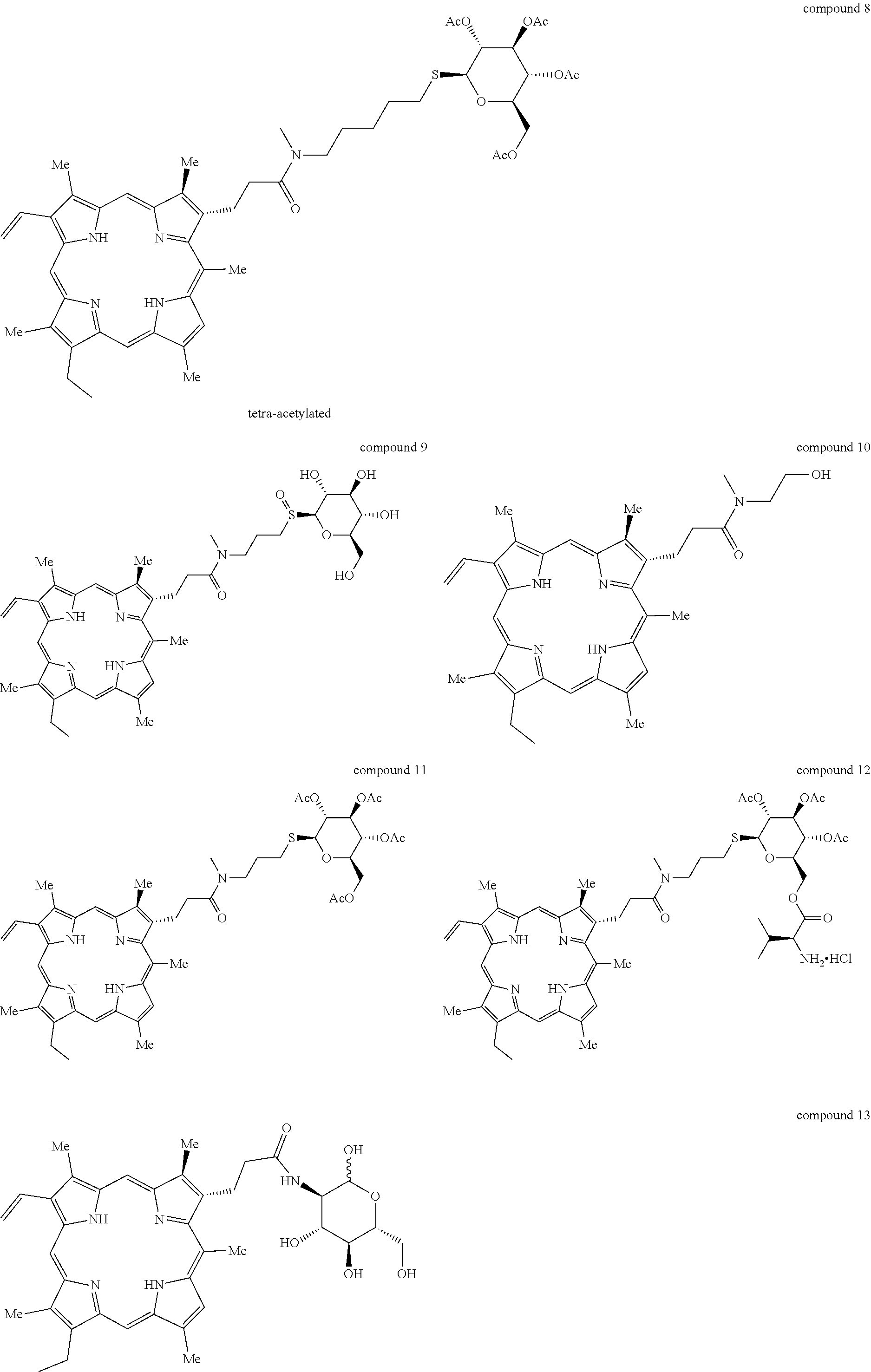

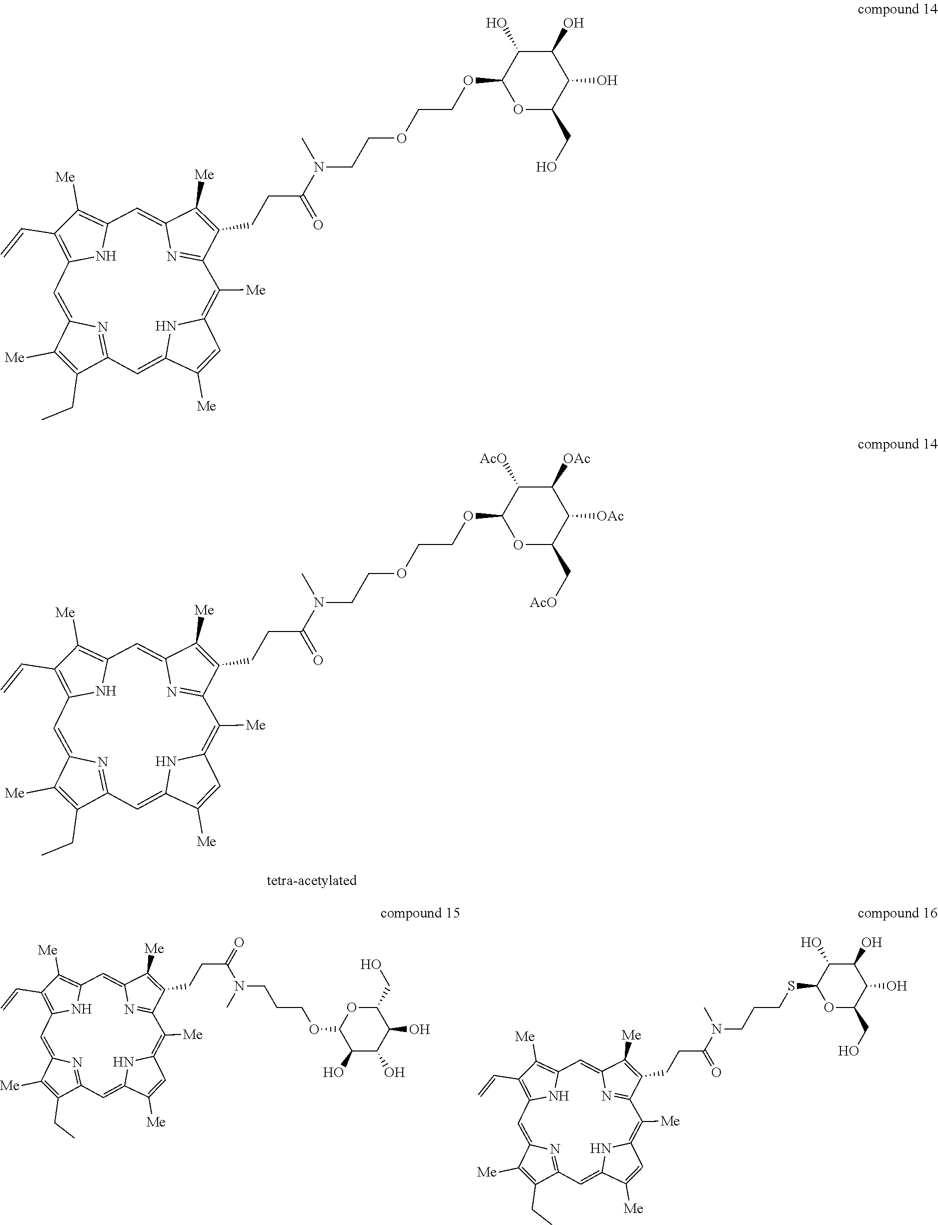

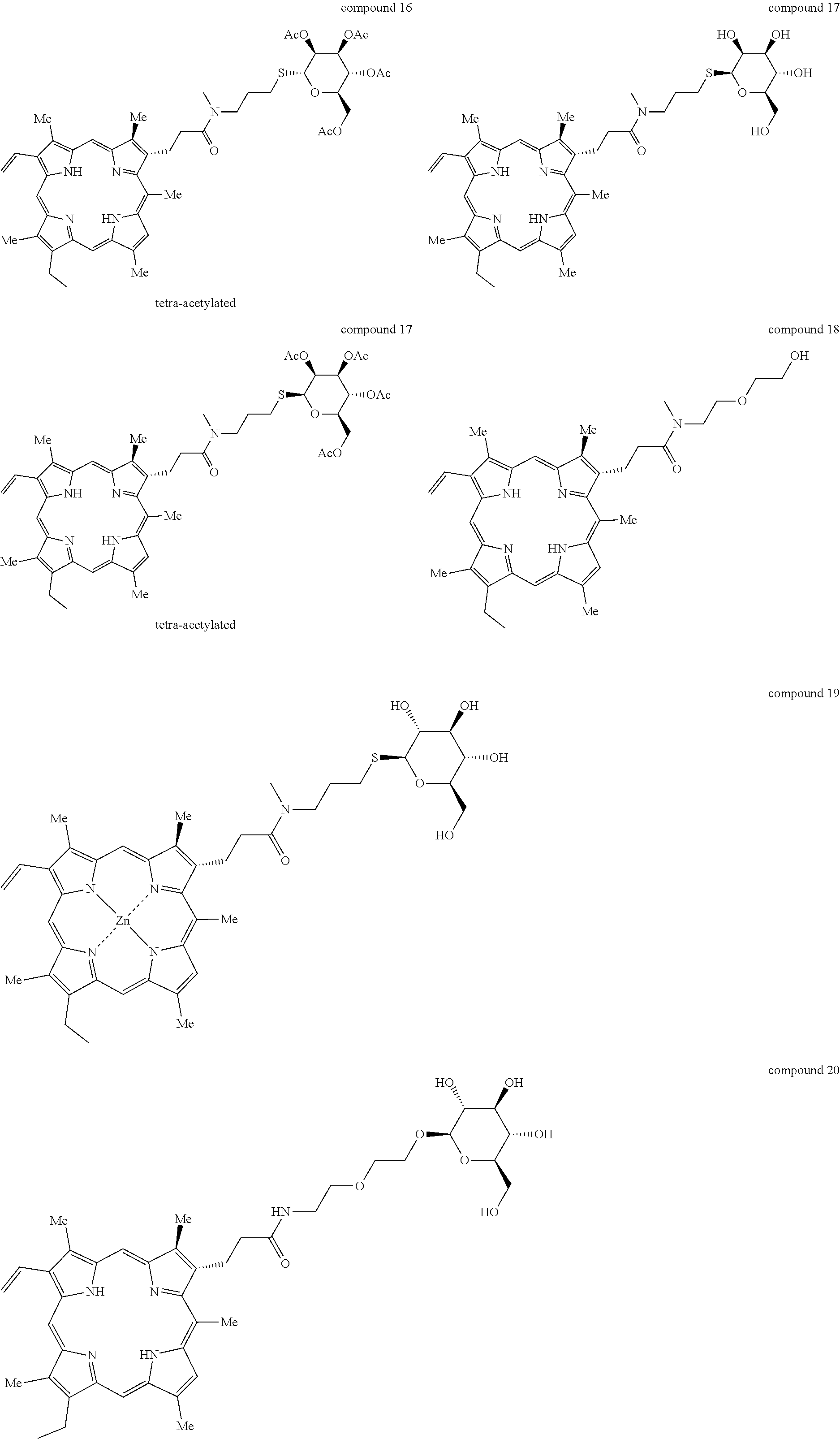

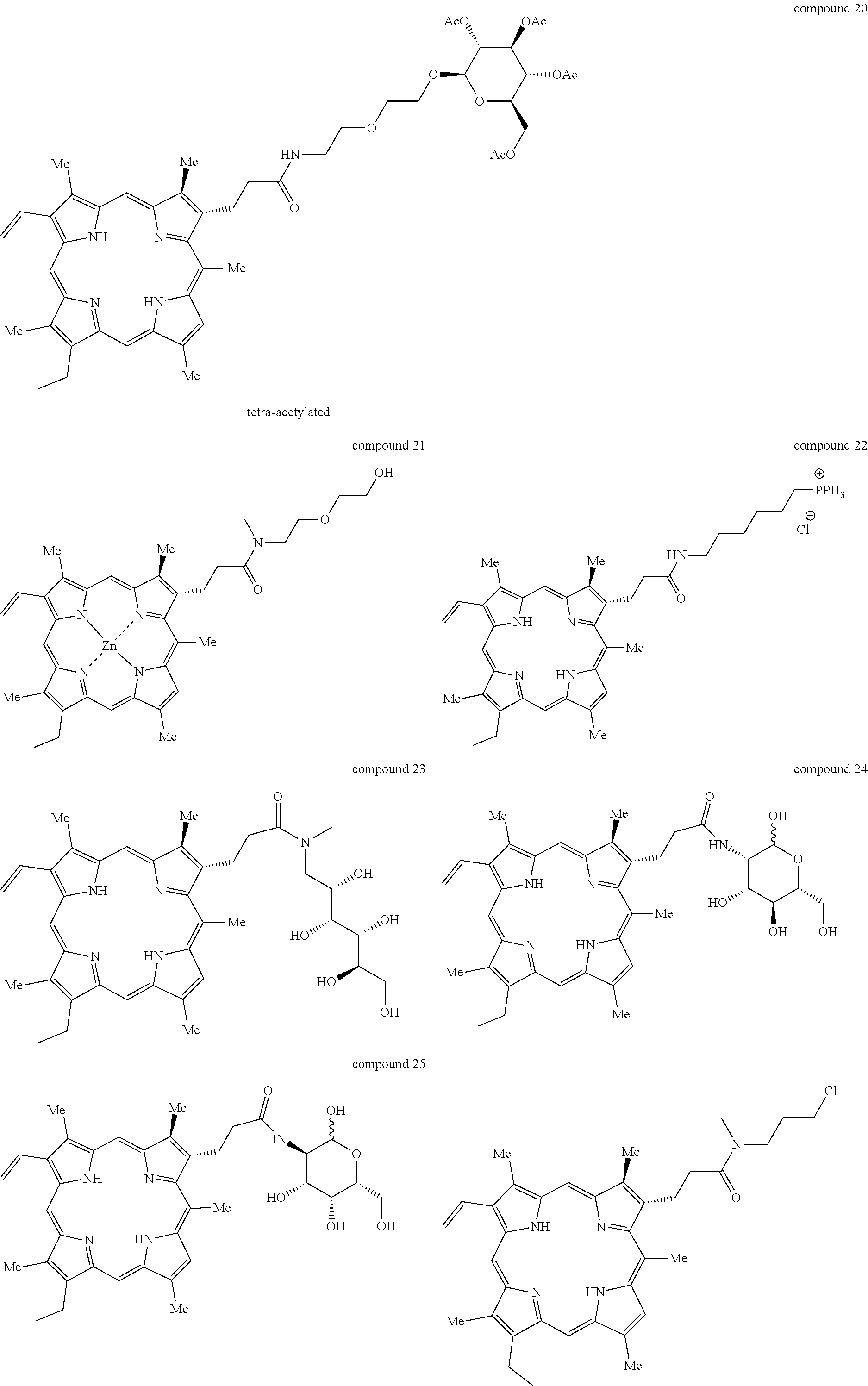

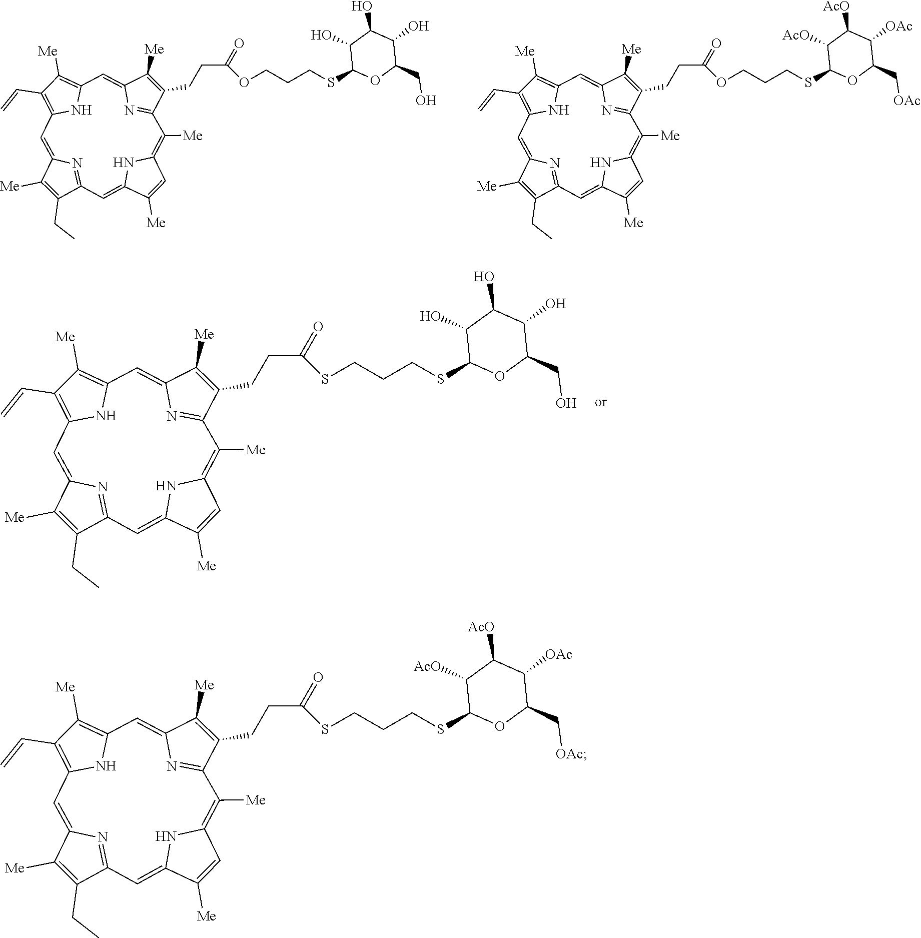

or a complex or a pharmaceutically acceptable salt thereof.

In one embodiment, the compound or complex according to the first or second aspect of the invention is in the form of a pharmaceutically acceptable salt. In one embodiment, the compound or complex is in the form of an inorganic salt such as a lithium, sodium, potassium, magnesium, calcium or ammonium salt. In one embodiment, the compound or complex is in the form of a sodium or potassium salt. In one embodiment, the compound or complex is in the form of a sodium salt. In another embodiment, the compound or complex is in the form of an organic salt such as an amine salt (for example a choline or meglumine salt) or an amino acid salt (for example an arginine salt).

In one embodiment, the compound or complex according to the first or second aspect is phyllochlorin in the form of a pharmaceutically acceptable salt. In one embodiment, the compound or complex is phyllochlorin in the form of a pharmaceutically acceptable inorganic salt such as a lithium, sodium, potassium, magnesium, calcium or ammonium salt. In one embodiment, the compound or complex is phyllochlorin mono-sodium or phyllochlorin mono-potassium. In one embodiment, the compound or complex is phyllochlorin mono-sodium. In another embodiment, the compound or complex is phyllochlorin in the form of a pharmaceutically acceptable organic salt such as an amine salt (for example a choline or meglumine salt) or an amino acid salt (for example an arginine salt).

The compound or complex according to the first or second aspect of the invention has at least two chiral centres. The compound or complex of the first or second aspect of the invention is preferably substantially enantiomerically pure, which means that the compound comprises less than 10% of other stereoisomers, preferably less than 5%, preferably less than 3%, preferably less than 2%, preferably less than 1% by weight, preferably less than 0.5% by weight, as measured by XRPD or SFC.

Preferably, the compound or complex according to the first or second aspect of the invention has a HPLC purity of more than 97%, more preferably more than 98%, more preferably more than 99%, more preferably more than 99.5%, more preferably more than 99.8%, and most preferably more than 99.9%. As used herein the percentage HPLC purity is measured by the area normalisation method.

A third aspect of the invention provides a composition comprising a compound or complex according to the first or second aspect of the invention and a pharmaceutically acceptable carrier or diluent.

In one embodiment, the composition according to the third aspect of the invention further comprises polyvinylpyrrolidone (PVP). In one embodiment, the composition comprises 0.01-10% w/w PVP as percentage of the total weight of the composition, preferably 0.1-5% w/w PVP as a percentage of the total weight of the composition, preferably 0.5-5% w/w PVP as a percentage of the total weight of the composition. In so one embodiment, the PVP is K30.

In one embodiment, the composition according to the third aspect of the invention further comprises dimethylsulfoxide (DMSO). In one embodiment, the composition comprises 0.01-99% w/w DMSO as percentage of the total weight of the composition, preferably 40-99% w/w DMSO as a percentage of the total weight of the composition, preferably 65-99% w/w DMSO as a percentage of the total weight of the composition.

In one embodiment, the composition according to the third aspect of the invention further comprises an immune checkpoint inhibitor. In one embodiment, the immune checkpoint inhibitor is an inhibitor of PD-1 (programmed cell death protein 1), PD-L1 (programmed death ligand 1) or CTLA4 (cytotoxic T-lymphocyte associated protein 4). In one embodiment, the immune checkpoint inhibitor is selected from Pembrolizumab, Nivolumab, Cemiplimab, Atezolizumab, Avelumab, Durvalumab or Ipilimumab.

Preferably the compound or complex according to the first or second aspect of the present invention and the pharmaceutical composition according to the third aspect of the present invention are suitable for use in photodynamic therapy or cytoluminescent therapy.

Preferably the compound or complex according to the first or second aspect of the present invention and the pharmaceutical composition according to the third aspect of the present invention are suitable for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, so gall bladder, spleen, brain, lymphatic system, bones, skin or pancreas.

Preferably the compound or complex according to the first or second aspect of the present invention and the pharmaceutical composition according to the third aspect of the present invention are suitable for the treatment of a disease characterised by benign or malignant cellular hyperproliferation or by areas of neovascularisation.

Preferably the compound or complex according to the first or second aspect of the present invention and the pharmaceutical composition according to the third aspect of the present invention are suitable for the treatment of a benign or malignant tumour.

Preferably the compound or complex according to the first or second aspect of the present invention and the pharmaceutical composition according to the third aspect of the present invention are suitable for the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for use in photodynamic diagnosis.

Preferably the compound or complex according to the first or second aspect of the present invention and the pharmaceutical composition according to the third aspect of the present invention are suitable for the detection of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for the detection of an area that is affected by benign or malignant cellular hyperproliferation or by neovascularisation.

Preferably the compound or complex according to the first or second aspect of the present invention and the pharmaceutical composition according to the third aspect of the present invention are suitable for the detection of a benign or malignant tumour.

Preferably the compound or complex according to the first or second aspect of the present invention and the pharmaceutical composition according to the third aspect of the present invention are suitable for the detection of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas. Preferably the compound or complex according to the first or second aspect of the present invention and the pharmaceutical composition according to the third aspect of the present invention are suitable for the fluorescent or phosphorescent detection of the diseases listed above, preferably for the fluorescent or phosphorescent detection and quantification of the said diseases.

Preferably the compound or complex according to the first or second aspect of the present invention and the pharmaceutical composition according to the third aspect of the present invention are adapted for administration simultaneous with or prior to administration of irradiation or sound, preferably for administration prior to administration of irradiation.

If the compound or complex according to the first or second aspect of the present invention or the pharmaceutical composition according to the third aspect of the present invention are for use in photodynamic therapy or cytoluminescent therapy, then they are preferably adapted for administration 5 to 100 hours before the irradiation, preferably 6 to 72 hours before the irradiation, preferably 24 to 48 hours before the irradiation.

If the compound or complex according to the first or second aspect of the present invention or the pharmaceutical composition according to the third aspect of the present invention are for use in photodynamic diagnosis, then they are preferably adapted for administration 3 to 60 hours before the irradiation, preferably 8 to 40 hours before the irradiation.

Preferably the irradiation used in the photodynamic therapy, cytoluminescent therapy or photodynamic diagnosis is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

The pharmaceutical composition according to the third aspect of the present invention may be in a form suitable for oral, parenteral (including intravenous, subcutaneous, intramuscular, intradermal, intratracheal, intraperitoneal, intratumoral, intraarticular, intraabdominal, intracranial and epidural), transdermal, airway (aerosol), rectal, vaginal or topical (including buccal, mucosal and sublingual) administration. The pharmaceutical composition may also be in a form suitable for administration by enema or for administration by injection into a tumour. Preferably the pharmaceutical composition is in a form suitable for oral, parenteral (such as intravenous, intraperitoneal, and intratumoral) or airway administration, preferably in a form suitable for oral or parenteral administration, preferably in a form suitable for oral administration.

In one preferred embodiment, the pharmaceutical composition is in a form suitable for oral administration. Preferably the pharmaceutical composition is provided in the form of a tablet, capsule, hard or soft gelatine capsule, caplet, troche or lozenge, as a powder or granules, or as an aqueous solution, suspension or dispersion. More preferably the pharmaceutical composition is provided in the form of an aqueous solution, suspension or dispersion for oral administration, or alternatively in the form of a freeze-dried powder which can be mixed with water before administration to provide an aqueous solution, suspension or dispersion for oral administration. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

In another preferred embodiment, the pharmaceutical composition is in a form suitable for parenteral administration. Preferably the pharmaceutical composition is in a form suitable for intravenous administration. Preferably the pharmaceutical composition is provided in the form of an aqueous solution for parenteral administration, or alternatively in the form of a freeze-dried powder which can be mixed with water before administration to provide an aqueous solution for parenteral administration. Preferably the pharmaceutical composition is an aqueous solution or suspension having a pH of from 6 to 8.5. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

In another preferred embodiment, the pharmaceutical composition is in a form suitable for airway administration. Preferably the pharmaceutical composition is provided in the form of an aqueous solution, suspension or dispersion for airway administration, or alternatively in the form of a freeze-dried powder which can be mixed with water before administration to provide an aqueous solution, suspension or dispersion for airway administration. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

A fourth aspect of the present invention provides use of a compound or complex according to the first or second aspect of the present invention in the manufacture of a medicament for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fourth aspect of the present invention also provides use of a compound or complex according to the first or second aspect of the present invention in the manufacture of a phototherapeutic agent for use in photodynamic therapy or cytoluminescent therapy. Preferably the phototherapeutic agent is suitable for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or so by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the medicament or the phototherapeutic agent of the fourth aspect of the present invention is suitable for the treatment of a disease characterised by benign or malignant cellular hyperproliferation or by areas of neovascularisation.

Preferably the medicament or the phototherapeutic agent of the fourth aspect of the present invention is suitable for the treatment of a benign or malignant tumour.

Preferably the medicament or the phototherapeutic agent of the fourth aspect of the present invention is suitable for the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fourth aspect of the present invention also provides use of a compound or complex according to the first or second aspect of the present invention in the manufacture of a photodiagnostic agent for use in photodynamic diagnosis.

Preferably the photodiagnostic agent of the fourth aspect of the present invention is suitable for the detection of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by so benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the photodiagnostic agent of the fourth aspect of the present invention is suitable for the detection of an area that is affected by benign or malignant cellular hyperproliferation or by neovascularisation.

Preferably the photodiagnostic agent of the fourth aspect of the present invention is suitable for the detection of a benign or malignant tumour.

Preferably the photodiagnostic agent of the fourth aspect of the present invention is suitable for the detection of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the photodiagnostic agent of the fourth aspect of the present invention is suitable for the fluorescent or phosphorescent detection of the said diseases, preferably the fluorescent or phosphorescent detection and quantification of the said diseases.

Preferably the medicament, the phototherapeutic agent or the photodiagnostic agent is adapted for administration simultaneous with or prior to administration of irradiation or sound, preferably for administration prior to administration of irradiation.

If the medicament or the phototherapeutic agent is for use in photodynamic therapy or cytoluminescent therapy, then it is preferably adapted for administration 5 to 100 hours before the irradiation, preferably 6 to 72 hours before the irradiation, preferably 24 to 48 hours before the irradiation.

If the photodiagnostic agent is for use in photodynamic diagnosis, then it is preferably adapted for administration 3 to 60 hours before the irradiation, preferably 8 to 40 hours before the irradiation.

Preferably the irradiation used in the photodynamic therapy, cytoluminescent therapy or photodynamic diagnosis is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

A fifth aspect of the present invention provides a method of treating atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas; the method comprising administering a therapeutically effective amount of a compound or complex according to the first or second aspect of the present invention to a human or animal in need thereof.

The fifth aspect of the present invention also provides a method of photodynamic therapy or cytoluminescent therapy of a human or animal disease, the method comprising administering a therapeutically effective amount of a compound or complex according to the first or second aspect of the present invention to a human or animal in need thereof. Preferably the human or animal disease is atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the method of the fifth aspect of the present invention is a method of treating benign or malignant cellular hyperproliferation or areas of neovascularisation.

Preferably the method of the fifth aspect of the present invention is a method of treating a benign or malignant tumour.

Preferably the method of the fifth aspect of the present invention is a method of treating early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fifth aspect of the present invention also provides a method of photodynamic diagnosis of a human or animal disease, the method comprising administering a diagnostically effective amount of a compound or complex according to the first or second aspect of the present invention to a human or animal. Preferably the human or animal disease is atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas. Preferably the human or animal disease is characterised by benign or malignant cellular hyperproliferation or by areas of neovascularisation. Preferably the human or animal disease is a benign or malignant tumour. Preferably the human or animal disease is early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas. Preferably the method of photodynamic diagnosis is suitable for the fluorescent or phosphorescent detection of the said diseases, preferably for the fluorescent or phosphorescent detection and quantification of the said diseases.

In any of the methods of the fifth aspect of the present invention, the human or animal is preferably further subjected to irradiation or sound simultaneous with or after the administration of the compound or complex according to the first or second aspect of the invention. Preferably the human or animal is subjected to irradiation after the administration of the compound or complex according to the first or second aspect of the invention.

If the method is a method of photodynamic therapy or cytoluminescent therapy, then the human or animal is preferably subjected to irradiation 5 to 100 hours after administration of the compound or complex according to the first or second aspect of the invention, preferably 6 to 72 hours after administration, preferably 24 to 48 hours after administration.

If the method is a method of photodynamic diagnosis, then the human or animal is preferably subjected to irradiation 3 to 60 hours after administration of the compound or complex according to the first or second aspect of the invention, preferably 8 to 40 hours after administration.

Preferably the irradiation is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

In any of the methods of the fifth aspect of the present invention, preferably the human or animal is a human.

A sixth aspect of the present invention provides a pharmaceutical combination comprising:
(a) a compound or complex according to the first or second aspect of the present invention; and
(b) an immune checkpoint inhibitor.

In one embodiment, the immune checkpoint inhibitor is an inhibitor of PD-1 (programmed cell death protein 1), PD-L1 (programmed death ligand 1) or CTLA4 (cytotoxic T-lymphocyte associated protein 4). In one embodiment, the immune checkpoint inhibitor is selected from Pembrolizumab, Nivolumab, Cemiplimab, Atezolizumab, Avelumab, Durvalumab or Ipilimumab.

Preferably, the combination of the sixth aspect is for use in the treatment of a disease, disorder or condition, wherein the disease, disorder or condition is responsive to PD-1, PD-L1 or CTLA4 inhibition. Preferably, the combination of the sixth aspect is for use in the treatment of cancer. In one embodiment, the cancer is melanoma, lung cancer (e.g. non small cell lung cancer), kidney cancer, bladder cancer, head and neck cancer, or Hodgkin's lymphoma.

The sixth aspect also provides a use of the combination of the sixth aspect of the invention in the manufacture of a medicament for the treatment of a disease, disorder or condition which is responsive to PD-1, PD-L1 or CTLA4 inhibition. The sixth aspect also provides a use of the combination of the sixth aspect of the invention in the manufacture of a medicament for the treatment of cancer. In one embodiment, the cancer is melanoma, lung cancer (e.g. non small cell lung cancer), kidney cancer, bladder cancer, head and neck cancer, or Hodgkin's lymphoma.

The sixth aspect of the invention also provides a method of treating a disease, disorder or condition which is responsive to PD-1, PD-L1 or CTLA4 inhibition, the method comprising administering a therapeutically effective amount of the combination of the sixth aspect of the present invention to a human or animal in need thereof. The sixth aspect of the invention also provides a method of treating cancer, the method comprising administering a therapeutically effective amount of the combination of the sixth aspect of the present invention to a human or animal in need thereof. In one embodiment, the cancer is melanoma, lung cancer (e.g. non small cell lung cancer), kidney cancer, bladder cancer, head and neck cancer, or Hodgkin's lymphoma.

For the combination of the sixth aspect of the invention, the compound or complex according to the first or second aspect of the invention, and the immune checkpoint inhibitor may be provided together in one pharmaceutical composition or separately in two pharmaceutical compositions. If provided in two pharmaceutical compositions, these may be administered at the same time or at different times.

Preferably the combination of the sixth aspect is adapted for administration simultaneous with or prior to administration of irradiation or sound, preferably for administration prior to administration of irradiation. In one embodiment, the combination of the sixth aspect is adapted for administration 5 to 100 hours before the irradiation, preferably 6 to 72 hours before the irradiation, preferably 24 to 48 hours before the irradiation.

Preferably the irradiation used in the photodynamic therapy or cytoluminescent therapy is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

For the avoidance of doubt, insofar as is practicable any embodiment of a given aspect so of the present invention may occur in combination with any other embodiment of the same aspect of the present invention. In addition, insofar as is practicable it is to be understood that any preferred or optional embodiment of any aspect of the present invention should also be considered as a preferred or optional embodiment of any other aspect of the present invention.

### BRIEF DESCRIPTION OF THE DRAWINGS

FIG. 1 shows the absorbance spectrum of phyllochlorin solutions. The % refers to the volume of DMSO; the remaining solvent is PBS (phosphate buffered saline).

FIG. 2 shows the absorbance spectrum of phyllochlorin solutions in the presence of PVP (1% w/v as a percentage of the total volume of the solution). The % refers to the volume of DMSO; the remaining solvent is PBS (phosphate buffered saline). FIG. 2 also shows a graph of % DMSO against % maximal absorbance for solutions comprising phyllochlorin and for solutions comprising phyllochlorin and 1% PVP w/v (as a percentage of the total volume of the solution).

FIG. 3 shows the cytotoxicity of chlorin e4 disodium without PVP, chlorin e4 disodium with 1% PVP w/v (as a percentage of the total volume of the solution), phyllochlorin without PVP, and phyllochlorin with 1% PVP w/v (as a percentage of the total volume of the solution).

FIG. 4 shows the phototoxicity of chlorin e4 disodium without PVP, chlorin e4 disodium with 1% PVP w/v (as a percentage of the total volume of the solution), phyllochlorin without PVP, and phyllochlorin with 1% PVP w/v (as a percentage of the total volume of the solution).

FIG. 5A shows the uptake and retention time for compounds 1 and 1c in vitro. SKOV3 ovarian cancer cells were incubated with compound 1 or 1c for up to 24 hours. Cellular uptake and loss over time were monitored using the intrinsic fluorescence of phyllochlorin. Each point was measured in triplicate; mean±SD in each case. FIG. 5B shows that there was no apparent difference in cellular localisation between compounds 1 and 1c.

FIG. 6A shows the uptake and retention time for compounds 1 and 2 in vitro. SKOV3 so ovarian cancer cells were incubated with compound 1 or 2 for up to 24 hours. Cellular uptake and loss over time were monitored using the intrinsic fluorescence of phyllochlorin. Each point was measured in triplicate; mean±SD in each case. FIG. 6B shows that compound 2 displayed a distinctly punctate distribution in cells, whereas compound 1 was diffusely distributed throughout the cytoplasm.

FIG. 7A shows that functionalisation with saccharidyl groups enhances cellular uptake of phyllochlorin analogues in vitro. SKOV3 ovarian cancer cells were incubated with compound 2, 6, 8 or 17 and cellular uptake monitored over a 4 hour period. Each point was measured in triplicate; mean±SD in each case. FIG. 7B shows that compound 2 displayed a distinctly punctate distribution in cells, whereas compounds 6, 8 and 17 were diffusely distributed throughout the cytoplasm.

FIG. 8 shows the localisation and retention of compound 6 in tumours. FIG. 8A details the quantitative analysis of tumour-associated fluorescence of compound 6 following oral, IV, and IT or IP administration. Tumours were either primary breast (upper panel) or disseminated peritoneal metastases (lower panel). n=3/group; mean±SD. FIG. 8B shows red fluorescence of compound 6 stimulated by blue light at autopsy. In primary tumour compound 6 was visualised as an intense red fluorescence when illuminated with blue light. In metastatic disease compound 6 was localised to individual metastatic nodules on multiple peritoneal surfaces and in a continuous omental mass consistent with metastatic ovarian cancer. FIG. 8C shows the localisation and retention of compound 6 compared with Talaporfin sodium and 5-ALA following IT injection in primary breast tumours.

FIG. 9 shows that photodynamic therapy with compound 6 resulted in complete regression of established tumours. Mice with established breast tumours were treated (day 6 post-implant) with compound 6 and laser (treated) or compound 6 alone (control). Tumour size was monitored until endpoint (tumour size ≥100 mm²). Treatment regressed established tumours to an undetectable level within 14 days of treatment. n=2/group; mean±SD.

FIG. 10 shows the ¹H NMR of compound 15.

### SYNTHETIC EXPERIMENTAL DETAILS

### Synthesis Example 1—Synthesis of Phyllochlorin Sodium Salt (Compound 1)

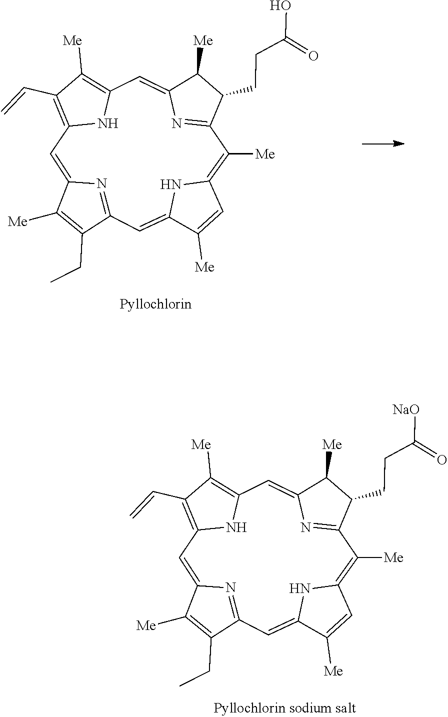

Into a 100 mL pear shaped RBF was weighed phyllochlorin (121 mg, 88.7% pure, 0.211 mmol) followed by distilled deionized water (15 mL) with swirling. Using a graduated pipette, sodium hydroxide solution (2.1 mL, 0.101 M, 1 eq) was added dropwise with hand swirling. The material was sonicated (10 minutes) to give a clear dark brown colour. The solution was subjected to freeze-drying overnight resulting in phyllochlorin sodium salt (compound 1) as a fine black powder (97 mg, 90%).

¹H NMR (400 MHz, d₆-DMSO) δ −2.42 (s, 1H), −2.26 (s, 1H), 1.62 (m, 1H), 1.69 (m, 6H), 2.12 (m, 1H), 2.43 (m, 2H), 3.32 (s, 3H), 3.48 (s, 3H), 3.60 (s, 3H), 3.81 (q, 2H), 3.98 (s, 3H), 4.59 (m, 2H), 6.14 (dd, 1H), 6.40 (dd, 1H), 8.31 (dd, 1H), 9.02 (s, 1H), 9.09 (s, 1H), 9.71 (s, 1H), 9.73 (s, 1H).

### Synthesis Example 1a—Synthesis of Phyllochlorin Potassium Salt (Compound 1a)

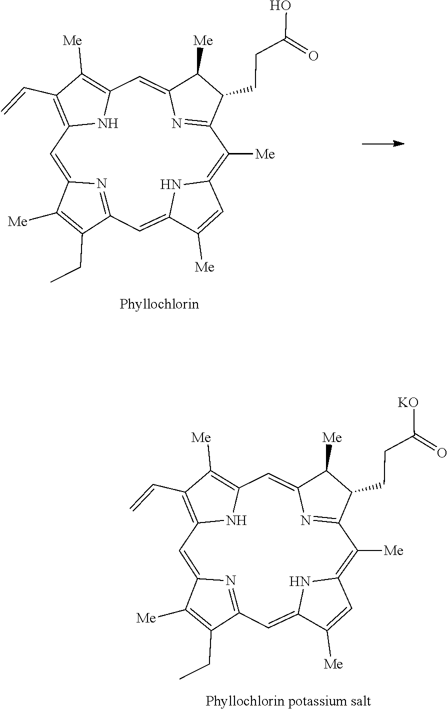

Into a 100 mL pear shaped RBF was weighed phyllochlorin (127 mg, 91.5% pure, 0.230 mmol) followed by distilled deionized water (15 mL) with stirring. Using a graduated pipette, potassium hydroxide solution (2.3 mL, 0.100 M, 1 eq) was added dropwise with stirring. The material was sonicated (15 minutes) to give a clear dark brown colour. The solution was subjected to freeze-drying overnight resulting in phyllochlorin potassium salt (compound 1a) as a fine black powder (127 mg, 93%).

¹H NMR (400 MHz, d₆-DMSO) δ 9.72 (s, 1H), 9.71 (s, 1H), 9.08 (s, 1H), 9.00 (s, 1H), 8.30 (dd, 1H), 6.40 (dd, 1H), 6.13 (dd, 1H), 4.57 (m, 2H), 3.96 (s, 3H), 3.80 (q, 2H), 3.60 (s, 3H), 3.48 (s, 3H), 3.31 (s, 3H), 2.40 (m, 2H), 2.10 (m, 1H), 1.69 (m, 6H), 1.60 (m, 1H), −2.27 (s, 1H), −2.43 (s, 1H).

### Synthesis Example 1b—Synthesis of Phyllochlorin Lithium Salt (Compound 1b)

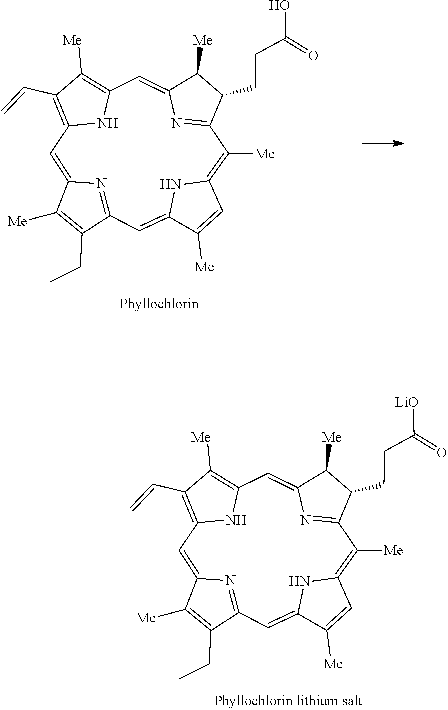

Into a 100 mL pear shaped RBF was weighed phyllochlorin (127 mg, 91.5% pure, 0.230 mmol) followed by distilled deionized water (15 mL) with stirring. Using a graduated pipette, lithium hydroxide solution (2.3 mL, 0.100 M, 1 eq) was added dropwise with stirring. The material was sonicated (15 minutes) to give a clear dark brown colour. The solution was subjected to freeze-drying overnight resulting in phyllochlorin lithium salt (compound 1b) as a fine black powder (115 mg, 90%).

¹H NMR (400 MHz, d₆-DMSO) δ 9.73 (s, 1H), 9.71 (s, 1H), 9.10 (s, 1H), 9.02 (s, 1H), 8.32 (dd, 1H), 6.43 (dd, 1H), 6.16 (dd, 1H), 4.59 (m, 2H), 3.98 (s, 3H), 3.81 (q, 2H), 3.60 (s, 3H), 3.51 (s, 3H), 3.32 (s, 3H), 2.35 (m, 2H), 2.00 (m, 1H), 1.69 (m, 6H), 1.62 (m, 1H), −2.26 (s, 1H), −2.43 (s, 1H).

### Synthesis Example 1c—Synthesis of Phyllochlorin Choline Salt (Compound 1c)

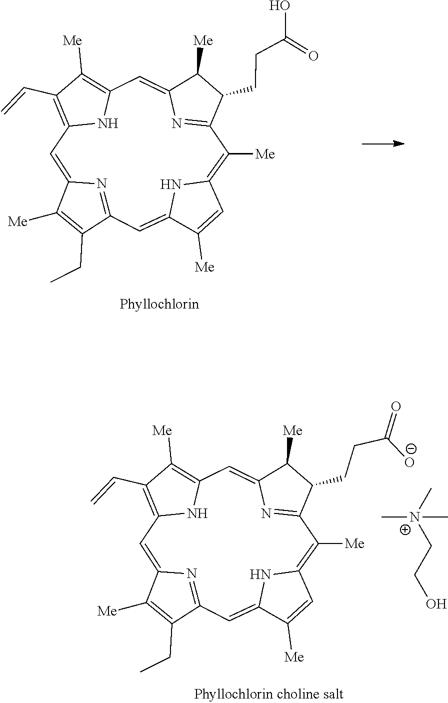

Into a 100 mL pear shaped RBF was weighed phyllochlorin (127 mg, 91.5% pure, 0.230 mmol) followed by distilled deionized water (15 mL) with stirring. Choline hydroxide (20% w/w in H₂O, 139 mg, 0.230 mmol, 1 eq) was added dropwise with stirring. The material was sonicated (5 minutes) to give a clear dark brown colour. The solution was subjected to freeze-drying overnight resulting in phyllochlorin choline salt (compound 1c) as a black powder (152 mg, quantitative).

¹H NMR (400 MHz, d₆-DMSO) δ 9.73 (s, 1H), 9.71 (s, 1H), 9.10 (s, 1H), 9.02 (s, 1H), 8.31 (dd, 1H), 6.41 (dd, 1H), 6.13 (dd, 1H), 4.58 (m, 2H), 3.98 (s, 3H), 3.81 (m, 4H), 3.60 (s, 3H), 3.51 (s, 3H), 3.38 (m, 2H), 3.31 (s, 3H), 3.09 (s, 12H), 2.38 (m, 2H), 2.05 (m, 1H), 1.69 (m, 6H), 1.61 (m, 1H), −2.26 (s, 1H), −2.42 (s, 1H).

### Synthesis Example 1d—Synthesis of Phyllochlorin Arginine Salt (Compound 1d)

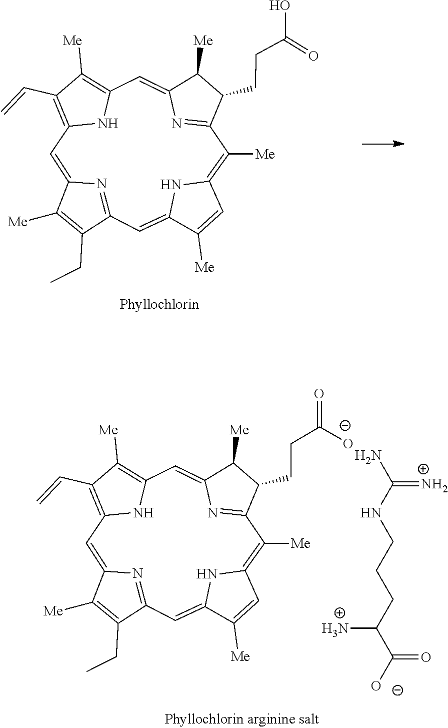 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; C=Cc1c(C)c2cc3nc(c(C)c4cc(C)c(cc5nc(cc1[nH]2)C(C)=C5CC)[nH]4)[C@@H](CCC(=O)[O-])[C@@H]3C ; NC(=[NH2+])NCCCC([NH3+])C(=O)[O-]]

Into a 100 mL pear shaped RBF was weighed phyllochlorin (127 mg, 91.5% pure, 0.230 mmol) followed by distilled deionized water (15 mL) with stirring. Arginine (40 mg, 0.230 mmol, 1 eq) was added. Further water (10 mL) was added and the solution was then heated at 60° C. for 1 hour. Acetone (2 mL) was added and stirring continued for 30 minutes at ambient temperature. The acetone was removed under reduced pressure and the remaining solution was subjected to freeze-drying overnight resulting in phyllochlorin arginine salt (compound 1d) as a black powder (140 mg, 82%).

¹H NMR (400 MHz, d₆-DMSO) δ 9.73 (s, 1H), 9.71 (s, 1H), 9.10 (s, 1H), 9.01 (s, 1H), 8.31 (dd, 1H), 8.20 (br s, 3H), 6.41 (dd, 1H), 6.13 (dd, 1H), 4.58 (m, 2H), 3.95 (s, 3H), 3.80 (m, 2H), 3.59 (m, 5H), 3.51 (s, 3H), 3.32 (s, 3H), 3.08 (m, 2H), 2.39 (m, 1H), 2.20 (m, 1H), 1.70 (m, 8H), 1.58 (m, 2H), −2.28 (s, 1H), −2.44 (s, 1H).

### Synthesis Example 1e—Synthesis of Phyllochlorin Meglumine Salt (Compound 1e)

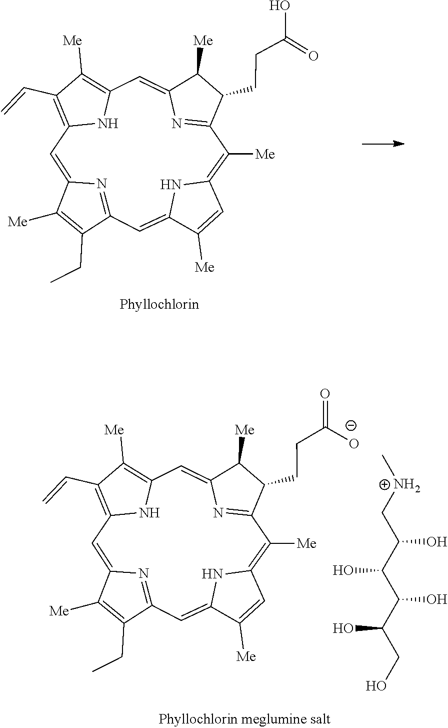 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; C=Cc1c(C)c2cc3nc(c(C)c4cc(C)c(cc5nc(cc1[nH]2)C(C)=C5CC)[nH]4)[C@@H](CCC(=O)[O-])[C@@H]3C ; C[NH2+]C[C@H](O)[C@@H](O)[C@H](O)[C@H](O)CO]

Into a 100 mL pear shaped RBF was weighed phyllochlorin (127 mg, 91.5% pure, 0.230 mmol) followed by distilled deionized water (15 mL) with stirring. Meglumine (45 mg, 0.230 mmol, 1 eq) was added. Further water (10 mL) was added and the solution was then heated at 60° C. for 1 hour. Acetone (2 mL) was added and stirring continued for 30 minutes at ambient temperature. The acetone was removed under reduced pressure and the remaining solution was subjected to freeze-drying overnight resulting in phyllochlorin meglumine salt (compound 1e) as a black powder (140 mg, 80%).

¹H NMR (400 MHz, d₆-DMSO) δ 9.73 (s, 1H), 9.71 (s, 1H), 9.10 (s, 1H), 9.02 (s, 1H), 8.32 (dd, 1H), 6.42 (dd, 1H), 6.16 (dd, 1H), 4.60 (m, 3H), 3.95 (s, 3H), 3.81 (m, 4H), 3.65 (m, 2H), 3.60 (m, 6H), 3.51 (s, 3H), 3.49 (m, 3H), 3.42-3.36 (m, 5H), 3.31 (s, 3H), 2.73 (m, 2H), 2.60 (m, 1H), 2.40 (m, 1H), 2.37 (s, 3H), 2.25 (m, 2H), 1.69 (m, 8H), −2.28 (s, 1H), −2.44 (s, 1H).

### Synthesis Example 2—Synthesis of Phyllochlorin Methyl Ester (Compound 2)

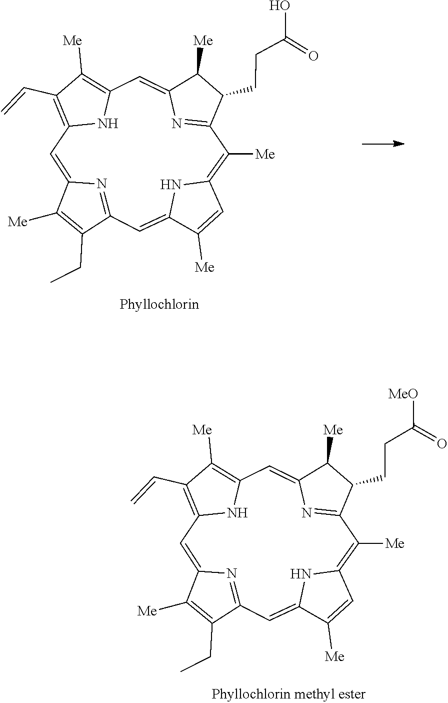 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; C=Cc1c(C)c2cc3nc(c(C)c4cc(C)c(cc5nc(cc1[nH]2)C(C)=C5CC)[nH]4)[C@@H](CCC(=O)OC)[C@@H]3C]

Into a single-neck 100 mL RBF was added phyllochlorin (2.40 g, 4.72 mmol, 1 eq), potassium carbonate (0.78 g, 5.66 mmol, 1.2 eq), DMF (30 mL) and a small sized stirrer bar. The flask was placed under N₂ and stirred at 300 rpm. Methyl iodide (382 μL, 6.13 mmol, 1.3 eq) was then added and the flask stirred at room temperature. HPLC analysis was undertaken after 2 hours and after stirring over the weekend, and confirmed the reaction was complete. The solution was diluted with DCM (30 mL) and filtered through Celite® (1 cm depth) washing with DCM until no more colour eluted. The solvent was removed under reduced pressure to give ˜4 g of a blue solid. The crude material was dissolved in EtOAc (125 mL) and washed with water (2×100 mL), dried (Na₂SO₄) and concentrated under reduced pressure to give crude product as a dark blue/green solid (2.7 g). The crude material was purified by column chromatography (silica, 4×23 cm, graduated solvent of DCM to 3% MeOH/DCM) to give phyllochlorin methyl ester (compound 2) as a dark blue/green solid (1.60 g, 64.8%).

¹H NMR (400 MHz, CDCl₃) δ −2.22 (br, 1H), −2.10 (br, 1H), 1.72-1.80 (m, 6H), 2.20-2.10 (m, 1H), 2.10-2.00 (m, 1H), 2.48-2.62 (m, 2H), 3.38 (s, 3H), 3.53 (s, 3H), 3.58 (s, 3H), 3.64 (s, 3H), 3.84 (q, 2H), 3.98 (s, 3H), 4.50 (q, 1H), 4.56 (d, 1H), 6.13 (dd, 1H), 6.37 (dd, 1H), 8.16 (dd, 1H), 8.83 (br s, 2H), 9.71 (br s, 2H).

### Synthesis Example 3—Synthesis of 3-((7S,8S)-18-ethyl-2,5,8,12,17-pentamethyl-13-vinyl-7H,8H-porphyrin-7-yl)-N-(3-(((2S,3R,4S,5S,6R)-3,4,5-trihydroxy-6-(hydroxymethyl)tetrahydro-2H-pyran-2-yl)thio)propyl)propanamide (Compound 3)

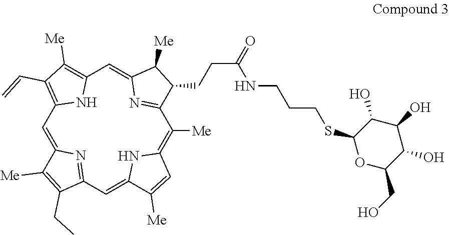 [SMILES: C=Cc1c(C)c2cc3nc(c(C)c4cc(C)c(cc5nc(cc1[nH]2)C(C)=C5CC)[nH]4)[C@@H](CCC(=O)NCCCS[C@@H]1O[C@H](CO)[C@@H](O)[C@H](O)[C@H]1O)[C@@H]3C]

### Synthesis of (2R,3R,4S,5R,6S)-2-(acetoxymethyl)-6-((3-aminopropyl)thio) tetrahydro-2H-pyran-3,4,5-triyl triacetate

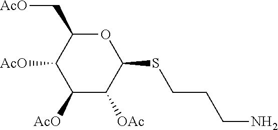 [SMILES: CC(=O)OC[C@H]1O[C@@H](SCCCN)[C@H](OC(C)=O)[C@@H](OC(C)=O)[C@@H]1OC(C)=O]

Step 1: A 2-neck 500 mL RBF fitted with a nitrogen inlet and rubber septa was charged with a solution of 1,2,3,4,6-penta-O-acetyl-β-D-glucose (6.23 g, 15.95 mol, 1 eq) in dry DCM (150 mL) and a stirrer bar, and the mixture was placed under N₂. To this solution was added (9H-fluoren-9-yl)methyl (3-mercaptopropyl)carbamate (ChemBioChem, 2010, 11(6), 778-781) (6.00 g, 19.1 mmol, 1.2 eq), before BF₃·OEt₂ (5.9 mL, 47.9 mmol, 3 eq) was added dropwise via the rubber septa over the course of 2-3 minutes. The mixture was stirred (315 rpm) at room temperature under N₂ overnight. TLC analysis at this point indicated only traces of starting material remaining. The reaction was quenched by the addition of 1 M HCl (50 mL) and transferred to a separatory funnel. The organic phase was collected and washed with brine (50 mL), before being dried (MgSO₄) and concentrated by rotary evaporation to give the crude glycosylated product as a lightly coloured syrup (18 g). The residue was purified by column chromatography (50% EtOAc/hexanes, loaded as a solution in the eluent, R_{f}=0.5) to give N-Fmoc-3′-amino-1-thio-2,3,4,6-tetra-O-acetyl-β-D-glucopyranose as a colourless syrup that solidified upon standing (5.55 g, 54%).

¹H NMR (400 MHz, CDCl₃) δ 7.76 (d, J=7.4 Hz, 2H), 7.60 (d, J=7.4 Hz, 2H), 7.39 (dd, J=7.4, 7.4 Hz, 2H), 7.31 (dd, J=7.4, 7.4 Hz, 2H), 5.22 (dd, J=9.4, 9.4 Hz, 1H), 5.05 (dd, J=9.4, 9.4 Hz, 1H), 5.02 (dd, J=9.4, 9.4 Hz, 1H), 4.93 (br s, 1H), 4.50-4.42 (m, 3H), 4.31-4.08 (m, 3H), 3.69 (ddd, J=10.1, 4.8, 2.7 Hz, 1H), 3.36-3.19 (m, 2H), 2.74 (ddd, J=13.4, 6.7, 6.7 Hz, 1H), 2.64 (ddd, J=13.4, 6.7, 6.7 Hz, 1H), 2.05 (s, 3H), 2.04 (s, 3H), 2.03 (s, 3H), 2.00 (s, 3H), 1.87-1.70 (m, 2H).

Step 2: To a 50 mL flask containing N-Fmoc-3′-amino-1-thio-2,3,4,6-tetra-O-acetyl-β-D-glucopyranose (633 mg, 0.983 mmol, 2 eq) and a stirrer bar was added 20% piperidine/DMF (15 mL), and the resultant solution was stirred (420 rpm) for 10 minutes under ambient atmosphere. An aliquot was taken and concentrated for 1H NMR analysis, which showed cleavage of the Fmoc group. The reaction mixture was concentrated and then reconstituted/concentrated from toluene five times (to remove all piperidine) to give (2R,3R,4S,5R,6S)-2-(acetoxymethyl)-6-((3-aminopropyl)thio)tetrahydro-2H-pyran-3,4,5-triyl triacetate as a gummy beige solid that was used without further purification.

### Synthesis of 3-((7S,8S)-18-ethyl-2,5,8,12,17-pentamethyl-13-vinyl-7H,8H-porphyrin-7-yl)-N-(3-(((2S,3R,4S,5S,6R)-3,4,5-trihydroxy-6-(hydroxymethyl)tetrahydro-2H-pyran-2-yl)thio)propyl)propanamide (Compound 3)

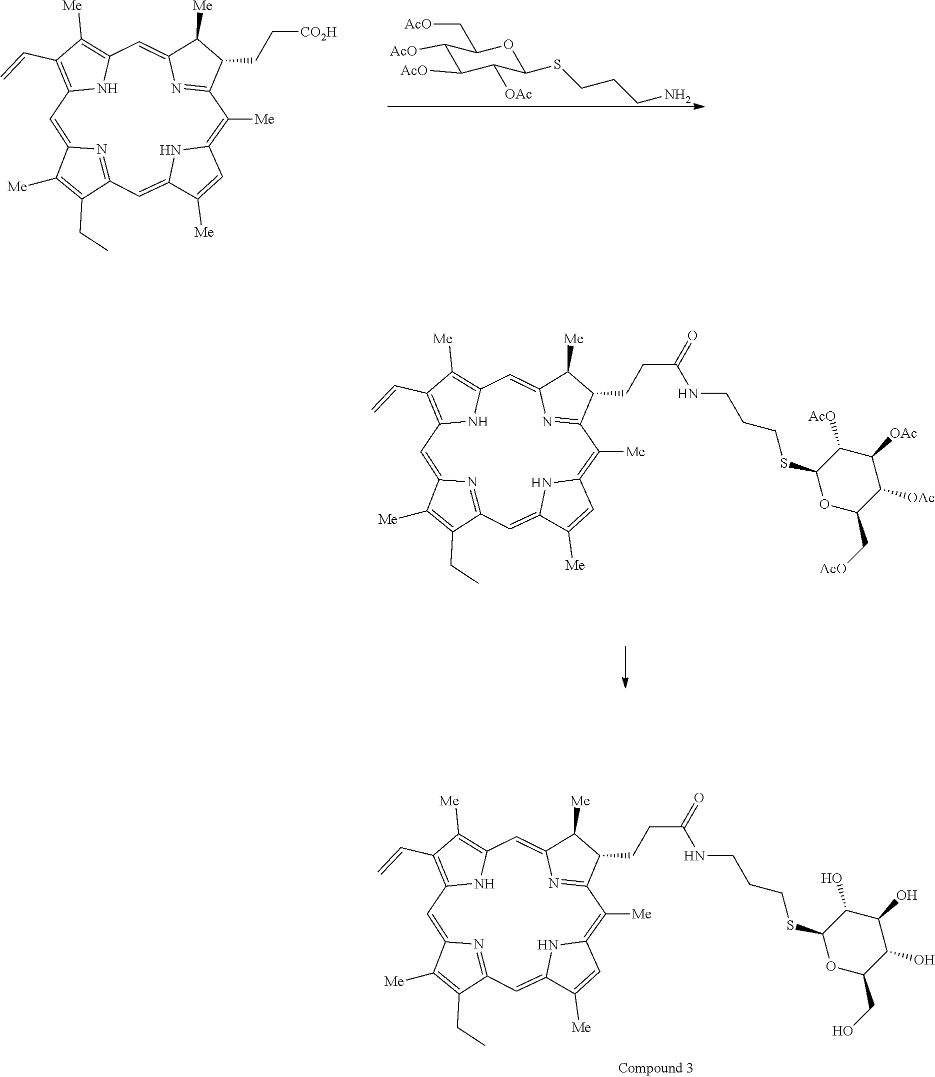 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; CC(=O)OCC1OC(SCCCN)C(OC(C)=O)C(OC(C)=O)C1OC(C)=O ; C=Cc1c(C)c2cc3nc(c(C)c4cc(C)c(cc5nc(cc1[nH]2)C(C)=C5CC)[nH]4)[C@@H](CCC(=O)NCCCS[C@@H]1O[C@H](COC(C)=O)[C@@H](OC(C)=O)[C@H](OC(C)=O)[C@H]1OC(C)=O)[C@@H]3C ; C=Cc1c(C)c2cc3nc(c(C)c4cc(C)c(cc5nc(cc1[nH]2)C(C)=C5CC)[nH]4)[C@@H](CCC(=O)NCCCS[C@@H]1O[C@H](CO)[C@@H](O)[C@H](O)[C@H]1O)[C@@H]3C]

Step 1: To a 50 mL RBF containing (2R,3R,4S,5R,6S)-2-(acetoxymethyl)-6-((3-aminopropyl)thio)tetrahydro-2H-pyran-3,4,5-triyl triacetate fitted with a nitrogen inlet was added phyllochlorin (250 mg, 0.491 mmol, 1 eq), DCM (12 mL) and a stirrer bar. To the resultant dark solution was added PyBOP (307 mg, 0.590 mmol, 1.2 eq), then triethylamine (204 μL, 1.47 mmol, 3 eq), and the mixture stirred (420 rpm) under N₂ for 1 hour. TLC analysis at 30 minutes indicated only traces of starting material remaining, as well the desired product (5% MeOH/DCM, R_{f} (starting material)=0.37, R_{f} (product)=0.70). The reaction mixture was transferred to a 100 mL separatory funnel and washed with 1 M HCl (2×20 mL) (the organic phase became a deep purple at this point), then pH 7 buffer (20 mL) (the organic phase reverted to a green colour). The organic phase was dried (Na₂SO₄) and concentrated by rotary evaporation to give the crude amide as a brown film (˜1.10 g). The residue was purified by Biotage autocolumn chromatography to give phyllochlorin β-1-thioglucose propylamide conjugate peracetate as a blue-black solid (417 mg, 95%). The crude material was deprotected without further purification.

Step 2: To a solution of phyllochlorin β-1-thioglucose propylamide conjugate peracetate (417 mg, 0.457 mmol, 1 eq) in MeOH (4 mL)/DCM (4 mL) was added NaOMe (4.6 M in MeOH, 0.50 mL, 2.286 mmol, 5 eq), and the mixture was stirred (420 rpm) under N₂ for 30 minutes. TLC analysis showed clean conversion to the deacetylated product (5% MeOH/DCM, R_{f} (starting material)=0.6, R_{f} (product)=0). The reaction was quenched with AcOH (5 drops) and concentrated by rotary evaporation. The residue was purified by column chromatography (3×17 cm, packed with 5% MeOH/DCM and using a gradient of 5-10% MeOH/DCM) to give compound 3 as a dark blue solid (255 mg, 70%—over 2 steps).

¹H NMR (400 MHz, d₆-DMSO) δ 9.75 (s, 1H), 9.73 (s, 1H), 9.10 (s, 1H), 9.08 (s, 1H), 8.33 (dd, 1H), 7.90 (t, 1H), 6.45 (d, 1H), 6.18 (d, 1H), 5.10 (d, 1H), 5.01 (d, 1H), 4.92 (d, 1H), 4.60 (m, 1H), 4.50 (m, 2H), 4.20 (d, 1H), 3.92 (s, 3H), 3.80 (m, 2H), 3.61 (s, 3H), 3.55 (s, 3H), 3.20-3.00 (m, 6H), 2.95 (m, 1H), 2.70-2.50 (m, 2H), 2.50-2.40 (m, 2H), 2.10 (m, 1H), 1.80-1.60 (m, 10H), −2.25 (s, 1H), −2.45 (s, 1H).

### Synthesis Example 4—Synthesis of 3-((7S,8S)-18-ethyl-2,5,8,12,17-pentamethyl-13-vinyl-7H,8H-porphyrin-7-yl)-N-(2-(2-(2-hydroxyethoxy)ethoxy)ethyl)-N-methylpropanamide (Compound 4)

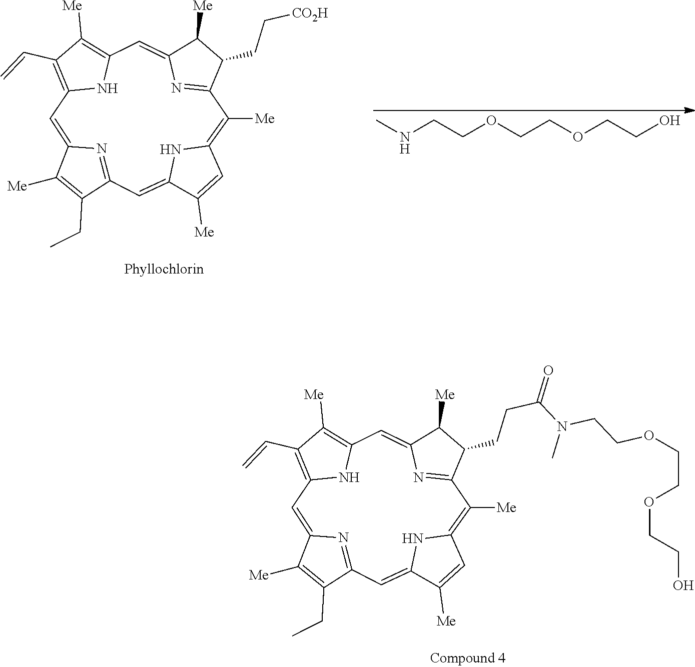 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; CNCCOCCOCCO ; C=Cc1c(C)c2cc3nc(c(C)c4cc(C)c(cc5nc(cc1[nH]2)C(C)=C5CC)[nH]4)[C@@H](CCC(=O)N(C)CCOCCOCCO)[C@@H]3C]

Into a 50 mL RBF fitted with a nitrogen inlet and containing a small stirrer bar was added phyllochlorin (500 mg, 0.983 mmol, 1 eq), dichloromethane (15 mL), PyBOP (563 mg, 1.1 eq), triethylamine (409 μL, 3 eq) and 2-(2-(2-(methylamino)ethoxy)ethoxy)ethanol (193 mg, 1.2 eq). The mixture was stirred at room temperature for 1 hour. Analysis by HPLC showed the reaction to be complete. The reaction mixture was transferred to a separatory funnel and washed with water (2×10 mL) and the organic layer dried (Na₂SO₄) and concentrated by rotary evaporation to give the crude product as a blue/brown film (1.10 g). The crude mixture was loaded directly onto a silica column and eluted with 3-7% MeOH/DCM. Pure fractions containing a green/blue compound by TLC(R_{f} 0.20 in 5% MeOH/DCM) were combined to give the final product, compound 4.

¹H NMR (400 MHz, CDCl₃) δ 9.71 (m, 2H), 8.83 (m, 2H), 8.16 (dd, 1H), 6.38 (dd, 1H), 6.13 (dd, 1H), 4.66 (m, 1H), 4.54 (m, 1H), 4.01 (s, 3H), 3.84 (q, 2H), 3.63 (s, 3H), 3.55-3.50 (m, 4H), 3.47-3.30 (m, 10H), 3.04 (m, 1H), 2.77-2.50 (m, 6H), 2.40-2.20 (m, 4H), 1.93-1.35 (m, 1H), 1.30-1.22 (m, 6H), −2.10 (br, 1H), −2.24 (br, 1H).

### Synthesis Example 5—Synthesis of 3-((7S,8S)-18-ethyl-2,5,8,12,17-pentamethyl-13-vinyl-7H,8H-porphyrin-7-yl)-N-(3-hydroxypropyl)-N-methylpropanamide (Compound 5)

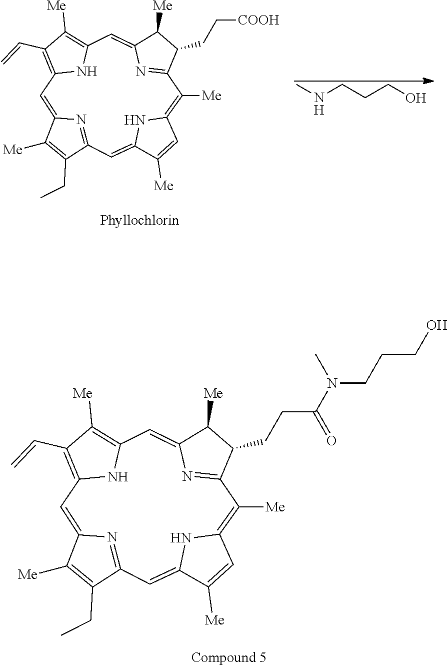

Into a 100 mL RBF fitted with a nitrogen inlet and containing a small stirrer bar was added phyllochlorin (2.00 g, 3.93 mmol, 1 eq), dichloromethane (50 mL), PyBOP (2.26 mg, 1.1 eq), triethylamine (1.64 mL, 3 eq) and 3-(methylamino)-propanol (0.42 g, 1.2 eq). The mixture was stirred at room temperature for 3 hours. Analysis by HPLC showed the reaction to be complete. The reaction mixture was transferred to a separatory funnel and washed with water (2×30 mL). The organic layer was dried (Na₂SO₄) and concentrated by rotary evaporation to give the crude product as a blue/brown film (5.1 g). The crude mixture was loaded directly onto a silica column and eluted with 1.5-2% MeOH/DCM. Pure fractions containing a green/blue spot by TLC with R_{f} 0.30 (5% MeOH/DCM) were combined to give compound 5 (1.29 g, 57%) (HPLC purity: 86.8%).

¹H NMR (400 MHz, CDCl₃) δ 9.75-9.70 (m, 2H), 8.85 (br s, 1H), 8.16 (dd, 1H), 6.38 (dd, 1H), 6.15 (dd, 1H), 4.70-4.65 (m, 1H), 4.58-4.50 (m, 1H), 4.00 (s, 3H), 3.89-3.82 (m, 3H), 3.65 (s, 3H), 3.53 (s, 3H), 3.37 (s, 3H), 3.31-3.15 (m, 4H), 2.65-2.53 (m, 2H), 2.37-2.22 (m, 3H), 2.16 (3, 3H), 1.80-1.70 (m, 7H), 1.48-1.40 (m, 2H), −2.10 (s, 1H), −2.22 (m, 1H).

### Synthesis Example 6—Synthesis of 3-((7S,8S)-18-ethyl-2,5,8,12,17-pentamethyl-13-vinyl-7H,8H-porphyrin-7-yl)-N-methyl-N-(3-(((2S,3R,4S,5S,6R)-3,4,5-trihydroxy-6-(hydroxymethyl)tetrahydro-2H-pyran-2-yl)thio)propyl)propanamide (Also Called phyllochlorin β-D-1-thioglucose-N-methylpropylamide conjugate) (Compound 6)

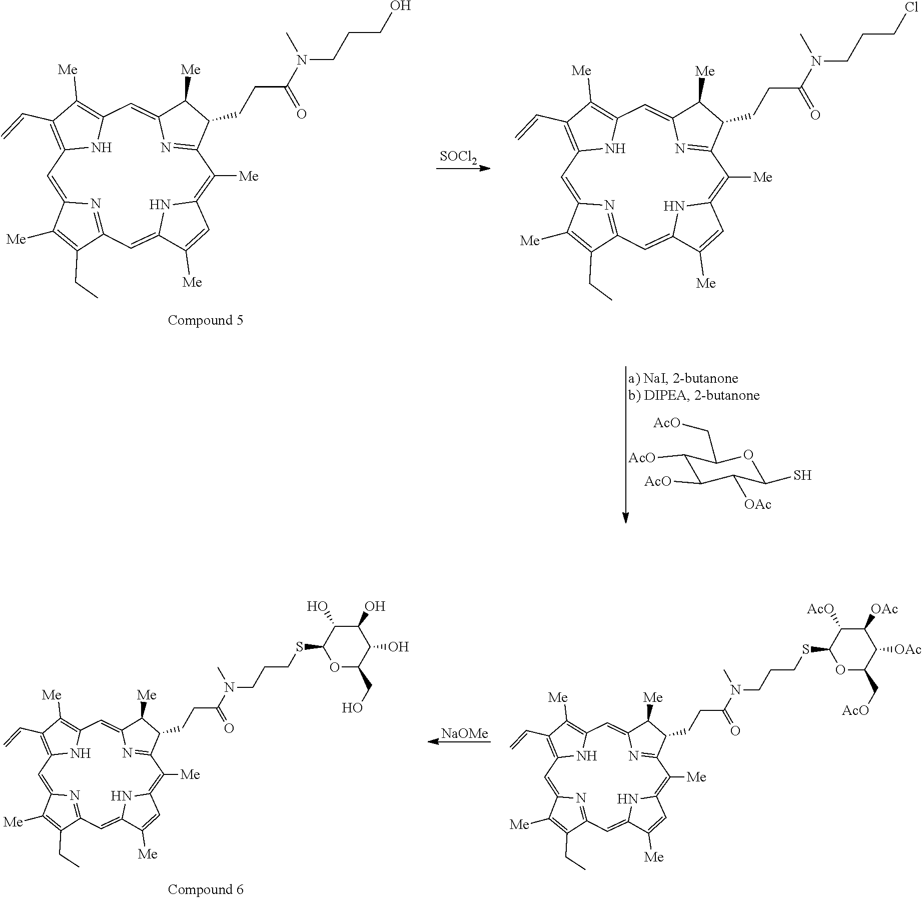

Step 1: A single-neck 50 mL RBF was charged with compound 5 (1.25 g, 2.156 mmol, 1 eq), dichloroethane (10 mL) and DMF (1 drop). Thionyl chloride (0.23 mL, 3.233 mmol, 1.5 eq) was added and the resultant solution was stirred (350 rpm) under N₂ at 40° C. After 2 hours, the flask was heated at 60° C. for a further 2 hours. The reaction was then cooled using an ice/water bath and pH=7 phosphate buffer (10 mL) was added. The mixture was extracted with DCM (3×15 mL), dried (Na₂SO₄) and the solvent was removed under reduced pressure to give ˜1.4 g of the crude product. The residual blue solid was purified by column chromatography using 2-4% MeOH/DCM and fractions containing the first dark band to elute were combined to give phyllochlorin N-3-chloropropyl-N-methyl propylamide as a blue/green solid (0.875 g, 67.8%).

Step 2: A single-neck 25 mL RBF was charged with phyllochlorin N-3-chloropropyl-N-methyl propylamide (145 mg, 0.242 mmol, 1 eq), 2-butanone (5 mL) and sodium iodide (73 mg). The resultant solution was stirred (350 rpm) under N₂ at 90° C. TLC after 3 hours indicated that the reaction was complete and the flask was then cooled using an ice/water bath. To the crude iodide was added thioglucose tetraacetate (106 mg, 0.291 mmol, 1.2 eq) and DIPEA (38 mg, 0.291 mmol, 1.2 eq) and the solution was stirred at 25° C. overnight. TLC analysis showed some starting material was present and the solution was heated at 50° C. for 3 hours. The solvent was removed and the residual blue solid was purified by column chromatography using 2-5% MeOH/DCM and fractions containing the darkest band (R_{f}˜0.6, 5% MeOH/DCM) were combined to give phyllochlorin β-1-thioglucose N-methyl propylamide conjugate peracetate as a blue/green solid (145 mg) that was used directly in the next step.

Step 3: To a solution of phyllochlorin β-1-thioglucose N-methyl propylamide conjugate peracetate (140 mg, 0.1512 mmol, 1 eq) in MeOH (2 mL)/DCM (2 mL) was added NaOMe (4.6 M in MeOH, 0.16 mL, 0.756 mmol, 5 eq), and the mixture was stirred (250 rpm) under N₂ for 30 minutes. TLC analysis showed conversion to the deacetylated product (5% MeOH/DCM, R_{f} (starting material)=0.6, R_{f} (product)=0). The reaction was quenched with acetic acid (10 drops) and concentrated by rotary evaporation. The residue was purified by column chromatography (2-8% MeOH/DCM) to elute compound 6 as a dark blue solid (30 mg, 16%—over 2 steps from the chloride).

¹H NMR (400 MHz, d₆-DMSO) δ 9.76 (s, 1H), 9.74 (s, 1H), 9.10 (d, J=5-7 Hz, 1H), 9.07 (s, 1H), 8.35 (dd, J=17.8, 11.6 Hz, 1H), 6.45 (dd, J=17.8, 1.6 Hz, 1H), 6.18 (dd, J=11.6, 1.5 Hz, 1H), 5.24-4.89 (m, 3H), 4.66 (p, J=7.4 Hz, 1H), 4.59-4.44 (m, 2H), 4.28 (dd, J=32.2, 9.6 Hz, 1H), 3.98 (d, J=6.1 Hz, 3H), 3.83 (q, J=7.6 Hz, 2H), 3.63 (d, J=1.0 Hz, 3H), 3.55 (d, J=1.6 Hz, 3H), 3.26-3.01 (m, 3H), 2.90 (s, 2H), 2.81 (s, 1H), 2.72-2.52 (m, 1H), 2.42 (dt, J=20.1, 5.8 Hz, 1H), 1.81 (ddd, J=22.3, 14.6, 6.9 Hz, 1H), 1.75-1.61 (m, 7H), −2.26 (s, 1H), −2.42 (d, J=2.2 Hz, 1H).

### Synthesis Example 7—Synthesis of 3-((7S,8S)-18-ethyl-2,5,8,12,17-pentamethyl-13-vinyl-7H,8H-porphyrin-7-yl)-N-(5-hydroxypentyl)-N-methylpropanamide (Also Called phyllochlorin N-5-hydroxypentyl-N-methyl propylamide) (Compound 7)

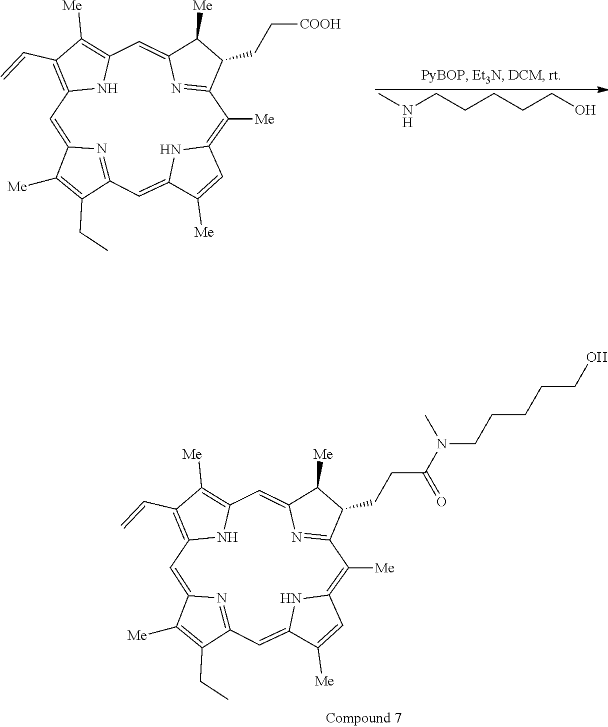

Into a 100 mL RBF fitted with a nitrogen inlet and containing a small stirrer bar was added phyllochlorin (0.87 g, 1.71 mmol, 1 eq), dichloromethane (25 mL), PyBOP (0.98 g, 1.1 eq), triethylamine (0.71 mL, 3 eq) and 5-(methylamino)-pentanol (0.24 g, 1.2 eq). The mixture was stirred at room temperature for 90 minutes. Analysis by HPLC showed the reaction to be complete. The reaction mixture was transferred to a separatory funnel and washed with water (2×30 mL). The organic layer was dried (Na₂SO₄) and concentrated by rotary evaporation to give the crude product as a blue/green film (2.1 g). The crude mixture was loaded directly onto a silica column (3×22 cm, pre-equilibrated with 1% MeOH/DCM) and eluted with the same solvent until the first pale-coloured band eluted and then the solvent was changed to 1.5% MeOH/DCM. When the most intense blue/green band began to elute the solvent was changed to 2% MeOH/DCM. Pure fractions containing a green/blue spot by TLC with R_{f} 0.30 (5% MeOH/DCM) were combined to give compound 7 (0.93 g, 89%) (HPLC purity: 98.2%).

¹H NMR (400 MHz, CDCl₃) δ 9.72 (br s, 2H), 8.87-8.82 (m, 1H), 8.16 (dd, 1H), 6.39 (dd, 1H), 6.15 (dd, 1H), 4.72-4.65 (m, 1H), 4.59-4.50 (m, 1H), 4.00 (s, 3H), 3.88-3.80 (m, 2H), 3.64 (s, 3H), 3.53 (m, 4H), 3.37 (s, 3H), 3.19-3.08 (m, 1H), 2.65-2.47 (m, 4H), 2.40-2.15 (m, 3H), 2.14-2.05 (m, 2H), 1.80-1.70 (m, 6H), 1.52-1.42 (m, 1H), 1.40-1.25 (m, 2H), 1.23-1.16 (m, 1H), 0.58-0.50 (m, 1H), 0.30-0.22 (m, 1H), 0.15-0.07 (m, 1H), −2.10 (s, 1H), −2.25 (m, 1H).

### Synthesis Example 8—Synthesis of 3-((7S,8S)-18-ethyl-2,5,8,12,17-pentamethyl-13-vinyl-7H,8H-porphyrin-7-yl)-N-methyl-N-(5-(((2S,3R,4S,5S,6R)-3,4,5-trihydroxy-6-(hydroxymethyl)tetrahydro-2H-pyran-2-yl)thio)pentyl)propanamide (Compound 8)

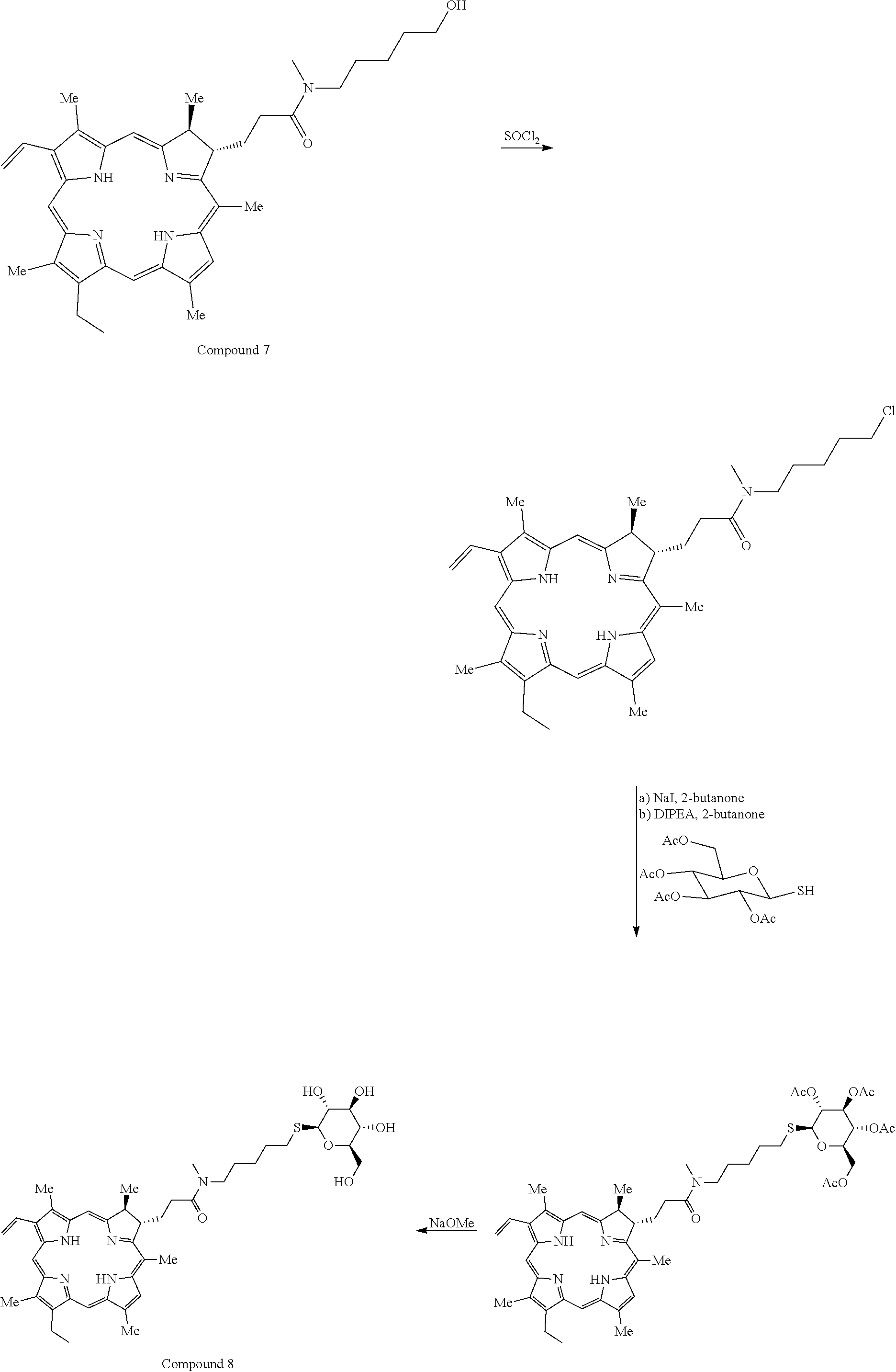

Step 1: A single-neck 50 mL RBF was charged with phyllochlorin N-5-hydroxypentyl-N-methyl propylamide (0.90 g, 1.481 mmol, 1 eq), dichloroethane (10 mL) and DMF (1 drop). Thionyl chloride (0.16 mL, 2.221 mmol, 1.5 eq) was added and the resultant solution was stirred (300 rpm) under N₂ at 40° C. TLC after 30 minutes indicated product (R_{f} 0.6, 5% MeOH/DCM) was the major compound present and the flask was heated at 55° C. for a further 30 minutes. The reaction was then cooled using an ice/water bath and pH=7 phosphate buffer (10 mL) was added. The mixture was extracted with DCM (3×10 mL), dried (Na₂SO₄) and the solvent was removed under reduced pressure to give the crude product. The residual blue solid was purified by column chromatography (3×20 cm of silica) using 1-4% MeOH/DCM. A brown band eluted first and then the product (R_{f}˜0.6-0.7) eluted when ˜3% MeOH/DCM was used. Fractions containing the dark band were combined to give phyllochlorin N-5-chloropentyl-N-methyl propylamide as a blue/green oily solid.

¹H NMR (400 MHz, CDCl₃) δ 9.72 (br s, 2H), 8.82 (br s, 2H), 8.16 (dd, 1H), 6.39 (m, 1H), 6.13 (d, 1H), 4.70-4.65 (m, 1H), 4.59-4.48 (m, 1H), 4.00 (s, 3H), 3.88-3.80 (m, 2H), 3.64 (s, 3H), 3.53 (m, 4H), 3.42 (t, 2H), 3.36 (s, 3H), 3.17-3.08 (m, 2H), 2.78 (t, 1H), 2.65-2.47 (m, 3H), 2.35-2.20 (m, 4H), 2.14-2.05 (m, 2H), 1.80-1.70 (m, 7H), 1.70-1.62 (m, 2H), 1.28-1.22 (m, 2H), 0.78-0.70 (m, 1H), 0.68-0.58 (m, 1H), 0.35-0.28 (m, 1H), −2.10 (s, 1H), −2.25 (m, 1H).

Step 2: A single-neck 250 mL RBF was charged with phyllochlorin N-5-chloropentyl-N-methyl propylamide (0.71 g, 1.13 mmol, 1 eq), 2-butanone (25 mL) and sodium iodide (340 mg). The resultant solution was stirred (350 rpm) under N₂ at 90° C. (external temperature, oil bath). TLC after 3 hours indicated that very little starting material was present and the flask was then cooled using an ice/water bath. To the crude iodide was so added thioglucose tetraacetate (496 mg, 1.36 mmol, 1.2 eq) and DIPEA (176 mg, 1.36 mmol, 1.2 eq) and the solution was stirred at 25° C. for 1 hour. TLC analysis showed some starting material was present and the solution was then heated at 40° C. for 1.5 hours. The solvent was removed and the crude product purified. The residual blue solid was purified by column chromatography (4×21 cm of silica) using 2-4% MeOH/DCM and fractions containing the darkest band (R_{f}˜0.5, 5% MeOH/DCM) were combined to give phyllochlorin β-1-thioglucose N-pentyl N-methyl propylamide conjugate peracetate as a blue/green solid (1.20 g).

Step 3: To a solution of phyllochlorin β-1-thioglucose N-pentyl N-methyl propylamide conjugate peracetate (1.05 g, 1.10 mmol, 1 eq) in MeOH (15 mL)/DCM (15 mL) was added NaOMe (4.6 M in MeOH, 1.20 mL, 5.50 mmol, 5 eq), and the mixture was stirred (250 rpm) under N₂ for 30 minutes. TLC analysis showed conversion to the deacetylated product (5% MeOH/DCM, R_{f} (starting material)=0.6, R_{f} (product)=0). The reaction was quenched with AcOH (30 drops) and concentrated by rotary evaporation. The residue was purified by column chromatography (4×18 cm, packed with 2% MeOH/DCM). After loading, the column was eluted sequentially using 5% MeOH/DCM (to elute high R_{f}), 6% MeOH/DCM (to elute mid R_{f}) and 8% MeOH/DCM to elute compound 8 as a dark blue solid (262 mg, 29%—over 3 steps from the chloride).

### Synthesis Example 9—Synthesis of 3-((7S,8S)-18-ethyl-2,5,8,12,17-pentamethyl-13-vinyl-7H,8H-porphyrin-7-yl)-N-methyl-N-(3-(((2S,3R,4S,5S,6R)-3,4,5-trihydroxy-6-(hydroxymethyl)tetrahydro-2H-pyran-2-yl)thio)propyl)propanamide sulphoxide (Compound 9)

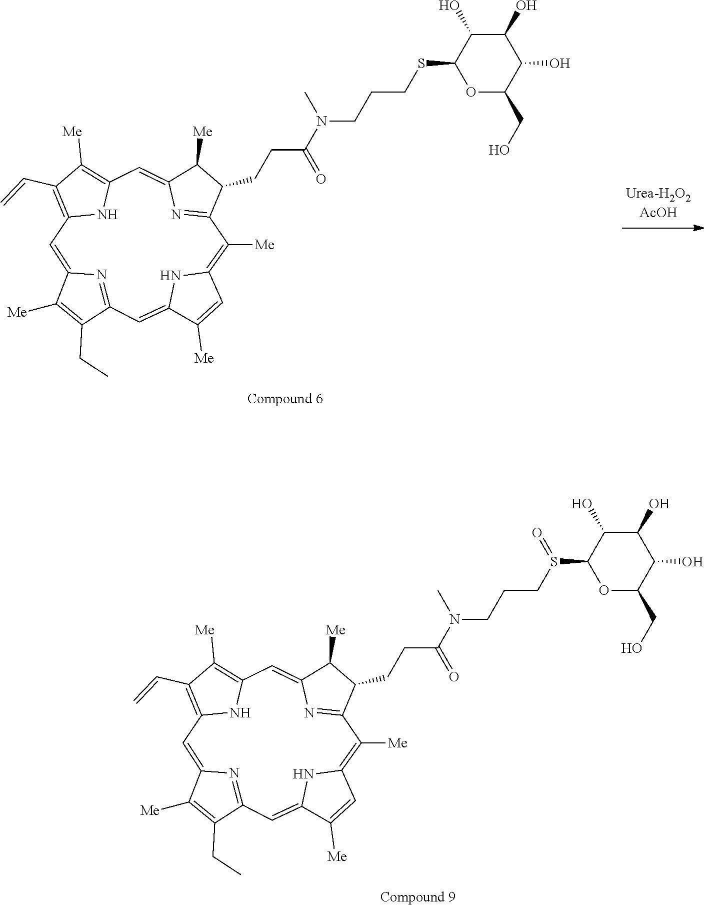 [SMILES: C=Cc1c(C)c2cc3nc(c(C)c4cc(C)c(cc5nc(cc1[nH]2)C(C)=C5CC)[nH]4)[C@@H](CCC(=O)N(C)CCCS[C@@H]1O[C@H](CO)[C@@H](O)[C@H](O)[C@H]1O)[C@@H]3C ; C=Cc1c(C)c2cc3nc(c(C)c4cc(C)c(cc5nc(cc1[nH]2)C(C)=C5CC)[nH]4)[C@@H](CCC(=O)N(C)CCCS(=O)[C@@H]1O[C@H](CO)[C@@H](O)[C@H](O)[C@H]1O)[C@@H]3C]

Into a single-neck 25 mL RBF was added phyllochlorin β-D-1-thioglucose-N-methylpropylamide conjugate (100 mg, 0.132 mmol, 1 eq), urea-hydrogen peroxide (18.6 mg, 0.198 mmol, 1.5 eq) and acetic acid (1.5 mL). The solution was stirred at 55° C. (external temperature, oil bath) for 2 hours. The acetic acid was removed under reduced pressure (rotary evaporator, 40° C., full vacuum) to leave a dark-green viscous oil. The crude product was purified by silica column chromatography (3×20 cm) using 10-16% MeOH/DCM. Fractions containing the product (R_{f} 0.3 in 10% MeOH/DCM) so were combined to give compound 9 as a dark green/blue flaky solid (91 mg, 89%).

¹H NMR (400 MHz, d₆-DMSO) δ 9.76-9.72 (m, 2H), 9.11-9.05 (m, 2H), 8.35 (dd, 1H), 6.44 (dd, 1H), 6.18 (dd, 1H), 5.70-5.30 (m, 1H), 5.24-5.02 (m, 2H), 4.80-4.60 (m, 2H), 4.60-4.52 (m, 1H), 4.31-4.10 (m, 1H), 3.99-3.95 (m, 3H), 3.81 (q, 2H), 3.75-3.65 (m, 1H), 3.62 (s, 3H), 3.55 (s, 3H), 3.51-3.39 (m, 3H), 3.33 (s, 3H), 3.17 (m, 1H), 3.16-2.97 (m, 2H), 2.90 (m, 2H), 2.84 (s, 1H), 2.82-2.55 (m, 2H), 2.47-2.38 (m, 1H), 1.99-1.75 (m, 2H), 1.73-1.66 (m, 7H), −2.25 (s, 1H), −2.43 (s, 1H).

### Synthesis Example 10—Synthesis of 3-((7S,8S)-18-ethyl-2,5,8,12,17-pentamethyl-13-vinyl-7H,8H-porphyrin-7-yl)-N-(2-hydroxyethyl)-N-methylpropanamide (Compound 10)

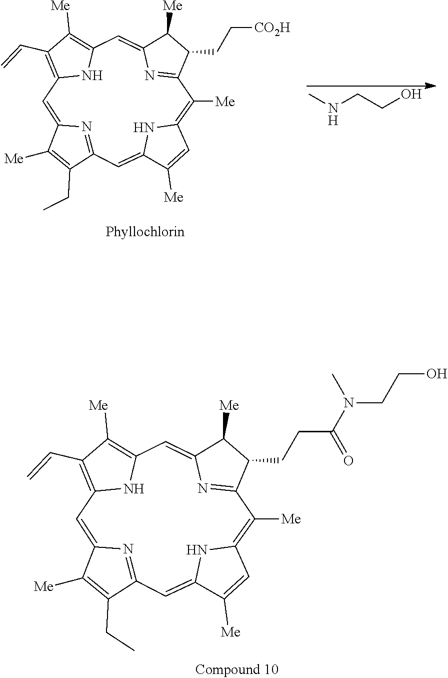

Into a 250 mL RBF fitted with a nitrogen inlet and containing a stirrer bar was added phyllochlorin (3.00 g, 5.90 mmol, 1 eq), dichloromethane (70 mL), PyBOP (3.30 g, 1.1 eq), triethylamine (2.46 mL, 3 eq) and 2-(methylamino)-ethanol (0.53 g, 1.2 eq). The mixture was stirred at room temperature for 3 hours. Analysis by TLC showed the reaction to be complete. The reaction mixture was transferred to a separatory funnel and washed with water (2×30 mL). The organic layer was dried (Na₂SO₄), filtered through Celite® and concentrated by rotary evaporation to give the crude product as a blue/brown film (6.0 g). The crude product was loaded directly onto a silica column and eluted with 1-3% MeOH/DCM. Pure fractions containing a green/blue spot by TLC (R_{f} 0.40 in 5% MeOH/DCM) were combined to give compound 10 (0.62 g, 25%) (HPLC purity: 83.8%).

¹H NMR (400 MHz, CDCl₃) δ 9.78-9.70 (m, 2H), 8.85 (m, 2H), 8.20-8.10 (m, 1H), 6.38 (d, 1H), 6.14 (d, 1H), 4.70-4.65 (m, 1H), 4.58-4.50 (m, 1H), 4.01 (m, 3H), 3.89-3.82 (m, 2H), 3.65 (s, 3H), 3.53 (s, 3H), 3.37 (m, 4H), 3.31-3.22 (m, 1H), 3.14-3.08 (m, 2H), 2.65-2.51 (m, 3H), 2.37-2.22 (m, 3H), 2.09 (s, 2H), 1.88-1.70 (m, 8H), 1.62-1.50 (br s, 2H), −2.05-−2.32 (m, 2H).

### Synthesis Example 11—Synthesis of (2R,3R,4S,5R,6S)-2-(acetoxymethyl)-6-((3-(3-((7S,8S)-18-ethyl-2,5,8,12,17-pentamethyl-13-vinyl-7H,8H-porphyrin-7-yl)-N-methylpropanamido)propyl)thio)tetrahydro-2H-pyran-3,4,5-triyl triacetate (Compound 11)

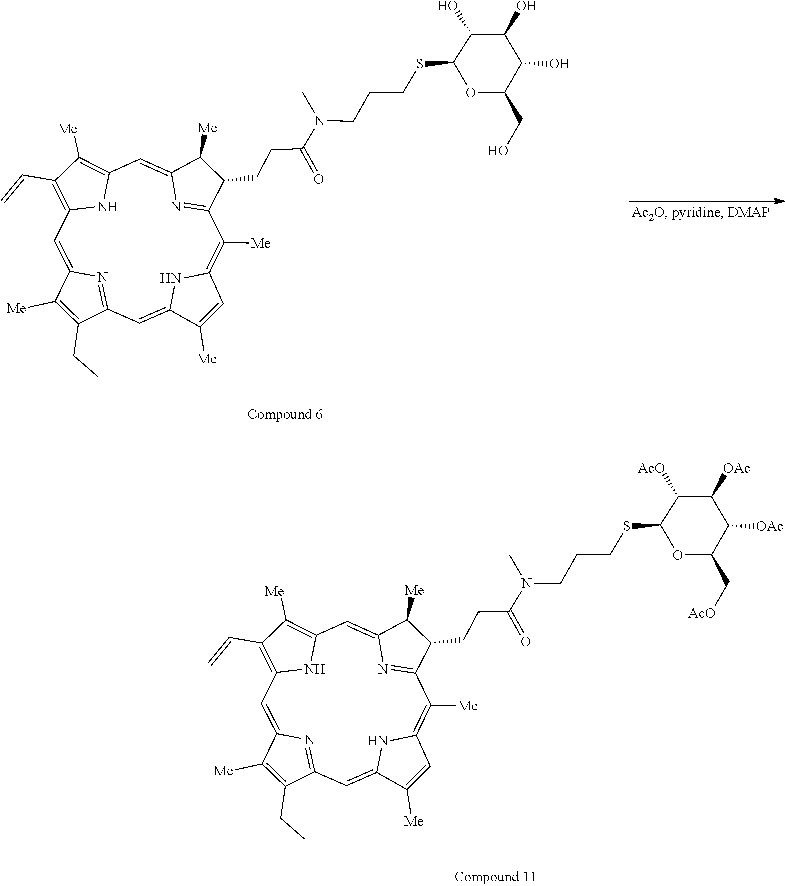 [SMILES: C=Cc1c(C)c2cc3nc(c(C)c4cc(C)c(cc5nc(cc1[nH]2)C(C)=C5CC)[nH]4)[C@@H](CCC(=O)N(C)CCCS[C@@H]1O[C@H](CO)[C@@H](O)[C@H](O)[C@H]1O)[C@@H]3C ; C=Cc1c(C)c2cc3nc(c(C)c4cc(C)c(cc5nc(cc1[nH]2)C(C)=C5CC)[nH]4)[C@@H](CCC(=O)N(C)CCCS[C@@H]1O[C@H](COC(C)=O)[C@@H](OC(C)=O)[C@H](OC(C)=O)[C@H]1OC(C)=O)[C@@H]3C]

Into a single-neck 100 mL RBF was added phyllochlorin β-D-1-thioglucose-N-methylpropylamide conjugate (2.00 g, 2.64 mmol, 1 eq), pyridine (15 mL), acetic anhydride (2.5 mL, 26.4 mmol, 10 eq) and DMAP (10 mg). The solution was stirred at 35° C. (external temperature, heat block) for 90 minutes. Analysis by TLC and HPLC indicated the reaction was complete. Ethyl acetate (40 mL) and water (30 mL) were added and the mixture stirred vigorously for 10 minutes. The layers were separated and the ethyl acetate layer washed with 0.5M HCl (4×30 mL), saturated NaHCO₃ (3×30 mL), dried (Na₂SO₄) and concentrated to give the crude product as a dark green solid (2.2 g). The crude product was purified by column chromatography (4×30 cm of silica) using 1-3% MeOH/DCM. Fractions containing the product (R_{f} 0.6 in 5% MeOH/DCM) were combined to give compound 11 as a dark green/blue flaky solid (2.25 g, 92%) (HPLC purity: 98.4%).

¹H NMR (400 MHz, CDCl₃) δ 9.72 (m, 2H), 8.90-8.85 (m, 2H), 8.23-8.11 (m, 1H), 6.45-6.35 (m, 1H), 6.19-6.15 (m, 1H), 5.12 (t, 0.5H), 5.01 (t, 0.5H), 4.93 (t, 0.5H), 4.78 (brt, 0.5H), 4.69 (br s, 0.5H), 4.60-4.50 (m, 1H), 4.30 (d, 0.5H), 4.20-4.05 (m, 1H), 4.01 (m, 4H), 3.92-3.78 (m, 2H), 3.65 (s, 3H), 3.54 (m, 4H), 3.41 (m, 3H), 3.25-3.08 (m, 1H), 2.75-2.65 (m, 0.5H), 2.60-2.50 (m, 4H), 2.50-2.35 (m, 1H), 2.30-2.15 (m, 3H), 2.00 (s), 1.97 (s), 1.94 (2×s), 1.88 (s), 1.83-1.72 (m, 9H), 1.50-1.40 (m, 1H), 1.35-1.20 (m, 1H), 0.90-0.70 (m, 1H), 0.50-0.25 (m, 1H), −2.10 (br, 1H), −2.25 (br, 1H).

### Synthesis Example 12—Synthesis of ((2R,3S,4S,5R,6S)-6-((3-(3-((7S,8S)-18-ethyl-2,5,8,12,17-pentamethyl-13-vinyl-7H,8H-porphyrin-7-yl)-N-methylpropanamido)propyl)thio)-3,4,5-trihydroxytetrahydro-2H-pyran-2-yl)methyl L-valinate hydrochloride (Compound 12)

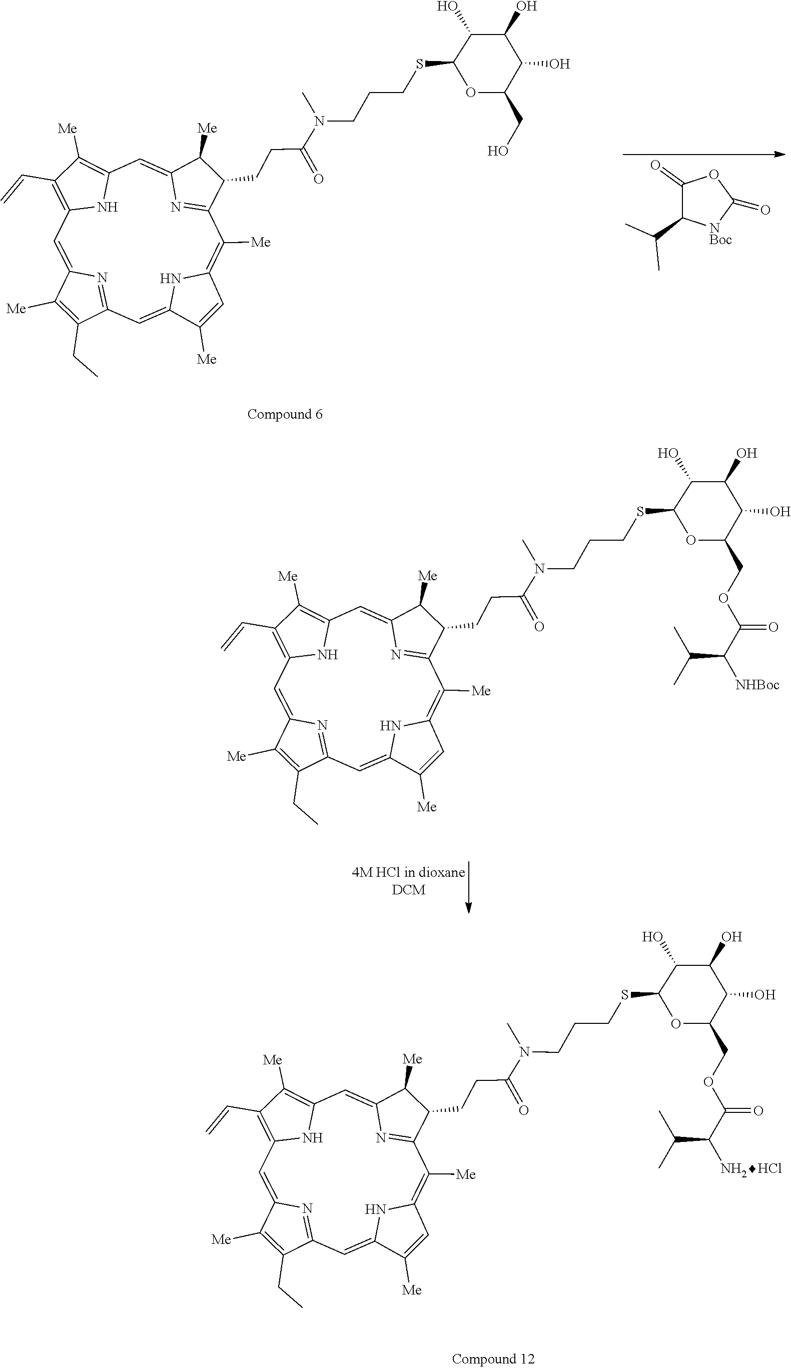

Step 1: Into a single-neck 25 mL RBF was added phyllochlorin β-D-1-thioglucose-N-methylpropylamide conjugate (283 mg, 0.374 mmol, 1 eq), N-Boc-L-valine-N-carboxyanhydride (100 mg, 0.411 mmol, 1.1 eq), DMF (6 mL) and DMAP (crystal). The solution was stirred at 350 rpm in the dark under nitrogen for 5 hours. The DMF was removed under reduced pressure (rotary evaporator, 50° C.) to leave a dark/green viscous oil which was purified by column chromatography (4×20 cm of silica) using 2-6% MeOH/DCM. Fractions containing the Boc-protected product (R_{f} 0.1-0.2 in 5% MeOH/DCM) were combined to give a dark green/blue oil (113 mg, 32%) (HPLC purity: 94% as mixture of regioisomers).

¹H NMR (400 MHz, CDCl₃) δ 9.74-9.69 (m, 2H), 8.94-8.83 (m, 2H), 8.20-8.08 (m, 1H), 6.46 (m, 1H), 6.14 (m, 1H), 5.10-4.95 (m, 1H), 4.75-4.50 (m, 2H), 4.27-4.05 (m, 2H), 4.03-3.97 (m, 3H), 3.82 (brq, 2H), 3.62 (m, 4H), 3.52 (m, 4H), 3.37-3.32 (m, 4H), 3.30-3.05 (m, 3H), 2.70-2.35 (m, 6H), 2.35-2.12 (m, 4H), 1.90-1.70 (m, 10H), 1.45-1.36 (m, 10H), 1.00-0.73 (m, 7H), −2.21-−2.45 (m, 2H).

Step 2: Into a single-neck 25 mL RBF was added the Boc-protected product from step 1 (113 mg, 0.118 mmol, 1 eq), 4M HCl in dioxane (0.3 mL, 1.20 mmol, 10 eq), dioxane (1 mL) and DCM (5 mL). The mixture was stirred in the dark under nitrogen at room temperature overnight. The solvent was removed under reduced pressure to leave a dark green/purple viscous oil which was purified by Biotage autocolumn chromatography (Reverse Phase, Sfar C18D 30 g column) using 5-50% MeCN/0.1M aqueous HCl. Fractions containing the product (R_{f} 0.2 in 20% MeOH/DCM) were combined to give compound 12 as a dark green/purple solid (41 mg, 39%) (HPLC purity: 97.7%).

### Synthesis Example 13—Synthesis of phyllochlorin 2-deoxyglucosamine-propylamide (Compound 13)

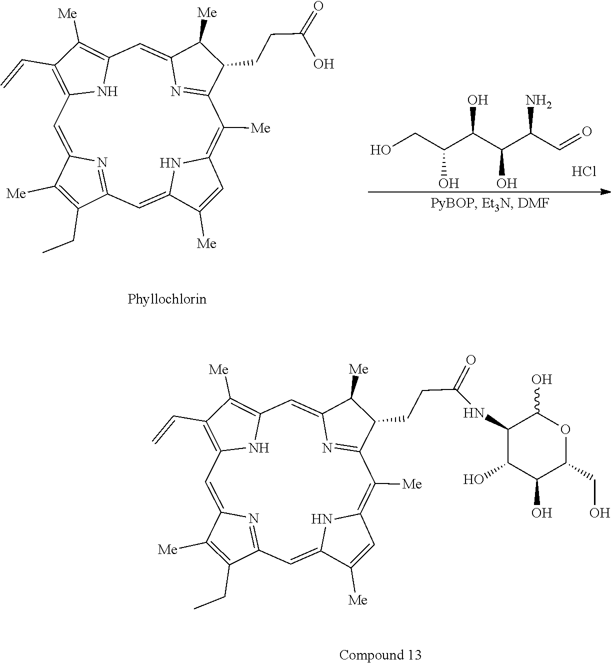 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; N[C@@H](C=O)[C@@H](O)[C@H](O)[C@H](O)CO ; Cl ; C=Cc1c(C)c2cc3nc(c(C)c4cc(C)c(cc5nc(cc1[nH]2)C(C)=C5CC)[nH]4)[C@@H](CCC(=O)N[C@H]1C(O)O[C@H](CO)[C@@H](O)[C@@H]1O)[C@@H]3C]

To a 10 mL RBF containing a stirrer bar was added phyllochlorin (200 mg, 0.3952 mmol, 1 eq), PyBOP (225 mg, 0.4325 mmol, 1.1 eq), DMF (4.0 mL) and triethylamine (120 μL, 0.8650 mmol, 2.2 eq). The resultant mixture was stirred (420 rpm) under nitrogen at ambient temperature for 5 minutes, then D-glucosamine hydrochloride (93 mg, 0.4325 mmol, 1.1 eq) was added in one portion. The resultant mixture was stirred for 30 minutes, by which time the reaction was complete as monitored by HPLC. The reaction mixture was concentrated by rotary evaporation to give a black residue which was dissolved in a minimum of DMSO and purified by Biotage autocolumn chromatography using a C-18 column. Fractions containing the product were combined to give compound 13 as a dark green/black solid (38.4 mg, 15%).

¹H NMR (400 MHz, d₆-DMSO) δ 9.76 (s, 1H), 9.74 (s, 1H), 9.13-9.06 (m, 2H), 8.36 (dd, J=17.8, 11.6 Hz, 1H), 7.74-7.66 (m, 1H), 6.50-6.42 (m, 1H), 6.31 (dd, J=4.5, 1.2 Hz, 1H), 6.18 (dd, J=11.6, 1.5 Hz, 1H), 4.92-4.84 (m, 2H), 4.67-4.34 (m, 4H), 3.94 (d, J=2.3 Hz, 3H), 3.83 (q, J=7.6 Hz, 2H), 3.68-3.58 (m, 4H), 3.58-3.50 (m, 4H), 3.50-3.39 (m, 2H), 3.15-2.98 (m, 1H), 2.46-2.31 (m, 1H), 1.79-1.64 (m, 7H), −2.27 (s, 1H), −2.43 (s, 1H). The product in solution exists as a mixture of epimers which causes two sets of signals in an ˜3:1 ratio.

### Synthesis Example 14—Synthesis of 3-((7S,8S)-18-ethyl-2,5,8,12,17-pentamethyl-13-vinyl-7H,8H-porphyrin-7-yl)-N-methyl-N-(2-(2-(((2R,3R,4S,5S,6R)-3,4,5-trihydroxy-6-(hydroxymethyl)tetrahydro-2H-pyran-2-yl)oxy)ethoxy)ethyl)propanamide (Compound 14)

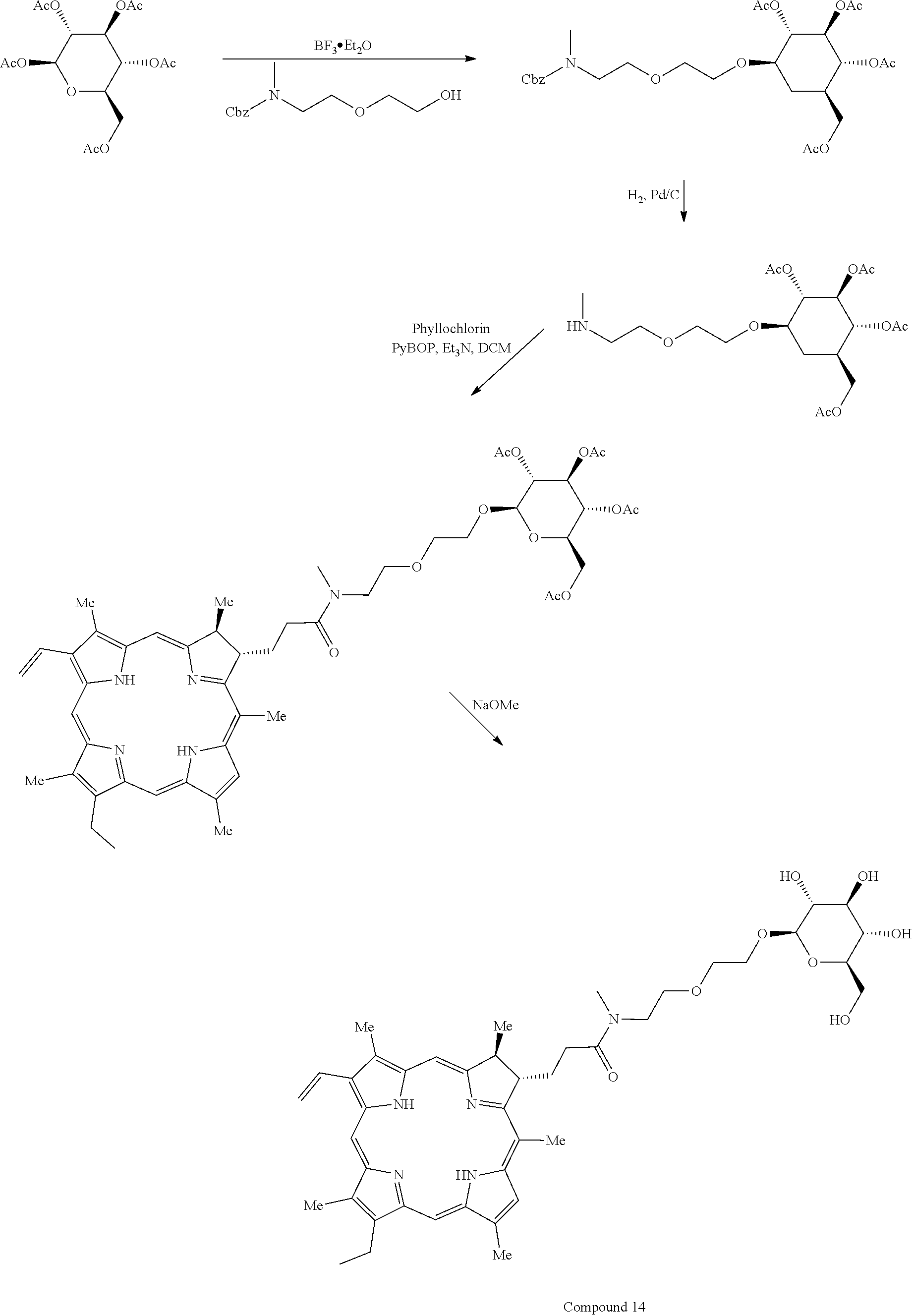

Step 1: A solution of N-Cbz-2-(2-methylamino-ethoxy)-ethanol (1.35 g, 5.33 mmol, 1 eq) and penta-acetyl glucose (2.29 g, 1.1 eq) in DCM (30 mL) under nitrogen was cooled in an ice/water bath and BF₃·Et₂O (3.78 g, 3.29 mL, 5 eq) was added dropwise via syringe. The solution was stirred (420 rpm) at 0-5° C. (external) for 1 hour and then at room temperature overnight. The reaction progress was checked by NMR. On completion of the reaction, the solution was washed with saturated NaHCO₃ (2×20 mL) and then the combined aqueous washes were extracted with DCM (20 mL). The so combined organic layers were dried (MgSO₄) and evaporated to give Cbz-protected PEG glucose as a pale yellow oil (˜4 g) which was partially purified by column chromatography (4×25 cm of silica) eluting using a gradient of 0.5-3.5% MeOH/DCM. The resulting oil (2.85 g) was used directly in the next step.

Step 2: To a 3-neck 250 mL RBF was added Cbz-protected PEG glucose (2.85 g), methanol (100 mL) and 10% Pd/C (140 mg, 5% w/w). A hydrogen balloon was attached to the flask and the flask was evacuated and re-filled with nitrogen three times. The flask was then evacuated and re-filled with hydrogen. The solution was stirred at 325 rpm overnight. After evacuating the flask and re-filling with nitrogen the solution was filtered (Celite®), washed with methanol (20 mL) and concentrated under reduced pressure. The concentrated residue was taken up in DCM (25 mL), washed with water (2×20 mL), dried (MgSO₄) and evaporated to give the amine PEG glucose (1.5 g) as a yellow oil which was used without further purification.

Step 3: To a 50 mL RBF was added phyllochlorin (1.04 g, 2.05 mmol, 1 eq), PyBOP (1.28 g, 2.46 mmol, 1.2 eq), DCM (15 mL) and triethylamine (1.92 mL, 13.9 mmol, 6.7 eq). The resultant mixture was stirred (250 rpm) under nitrogen at ambient temperature for 30 minutes, then the amine PEG glucose in DCM (10 mL) was added in one portion. The resultant mixture was stirred overnight in the dark under nitrogen. The reaction mixture was transferred to a separatory funnel and washed with 1M HCl (2×30 mL), then pH 7 buffer (1×30 mL). The organic phase was dried (Na₂SO₄) and concentrated by rotary evaporation to give a blue/black oil which was purified by column chromatography (4×26 cm of silica) eluting using a gradient of 1%→2.5% MeOH/DCM. Fractions containing phyllochlorin β-D-1-glucose-N-methyl ethoxyethyl amide conjugate tetraacetate (R_{f}˜0.4 in 5% MeOH/DCM) were combined to give a blue/black oil (˜0.7 g) which was further purified using Biotage autocolumn chromatography. Fractions with R_{f}-0.4 in 5% MeOH/DCM were combined to give phyllochlorin β-D-1-glucose-N-methyl ethoxyethyl amide conjugate tetraacetate (0.38 g) (HPLC purity: 88%) which was deprotected in the next step without further purification.

Step 4: To a solution of phyllochlorin β-D-1-glucose-N-methyl ethoxyethyl amide conjugate tetraacetate (350 mg, 0.372 mmol, 1 eq) in MeOH (5 mL) and DCM (5 mL) was added NaOMe (4.6M in MeOH, 0.40 mL, 1.862 mmol, 5 eq) and the mixture stirred (420 rpm) under nitrogen for 1 hour. TLC analysis showed conversion to the deacetylated product (10% MeOH/DCM, R_{f} (starting material)=0.85, R_{f} (product)=0.25). The reaction was quenched with AcOH (112 mg, 1.862 mmol, 5 eq) and concentrated by rotary evaporation to give a black film which was purified by column chromatography (3×21 cm of silica) eluting using a gradient of 3-10% MeOH/DCM to give compound 14 as a blue/black solid (204 mg, 71%) (HPLC purity: 91%).

¹H NMR (400 MHz, d₆-DMSO) δ 9.75 (s, 1H), 9.73 (s, 1H), 9.10 (s, 1H), 9.06 (s, 1H), 8.33 (dd, 1H), 6.44 (d, 1H), 6.18 (d, 1H), 5.30-4.70 (br m, 3H), 4.65 (m, 1H), 4.60-4.53 (m, 2H), 4.13 (m, 1H), 3.98 (d, 3H), 3.81 (m, 4H), 3.68-3.62 (m, 2H), 3.62 (s, 3H), 3.54 (d, 3H), 3.50-3.30 (m, 14H), 3.18-3.05 (m, 4H), 3.00-2.94 (m, 3H), 2.88-2.78 (m, 2H), 2.55-2.45 (m, 1H), 2.45-2.38 (m, 1H), 1.75-1.65 (m, 7H), −2.25 (s, 1H), −2.42 (s, 1H).

### Synthesis Example 15—Synthesis of 3-((7S,8S)-18-ethyl-2,5,8,12,17-pentamethyl-13-vinyl-7H,8H-porphyrin-7-yl)-N-methyl-N-(3-(((2R,3R,4S,5S,6R)-3,4,5-trihydroxy-6-(hydroxymethyl)tetrahydro-2H-pyran-2-yl)oxy)propyl)propanamide (Also Called phyllochlorin β-D-glucosyl-N-methylpropylamide conjugate) (Compound 15)

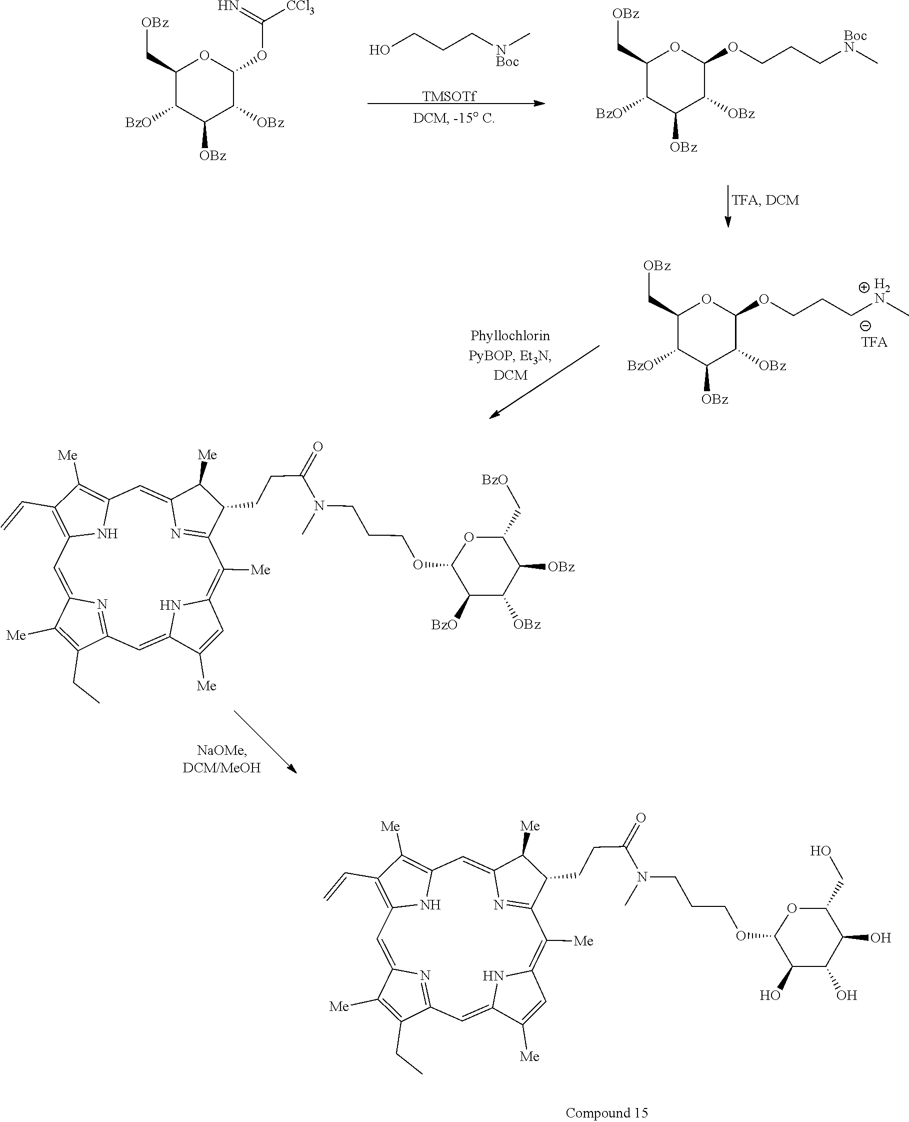

Step 1: A 250 mL 3-neck RBF fitted with an internal thermometer, nitrogen inlet and rubber septum was charged with tert-butyl (3-hydroxypropyl)(methyl)carbamate (2.68 g, 14.17 mmol, 1.05 eq), a stirrer bar, 2,3,4,6-tetra-O-benzoyl-α-D-glucopyranosyl trichloroacetimidate (10.00 g, 13.5 mmol, 1 eq), dry DCM (100 mL) and ground 4 A molecular sieves (5.0 g). The resultant suspension was placed under an atmosphere of nitrogen and stirred (420 rpm) for 0.5 hours before cooling to −15° C. (internal temperature) with the aid of an EtOH/ice/NaCl bath. A solution of TMSOTf (0.2M in DCM, 3.3 mL, 0.67 mmol) was added dropwise over the course of a minute, and stirring continued at low temperature for 0.5 hours, at which point TLC analysis indicated complete reaction (25% EtOAc/hexanes, R_{f} (starting material)=0.4, R_{f}(product)=0.2, visualised by UV). The reaction was quenched with triethylamine (8 drops) and filtered through Celite®, washing through with a further portion of DCM. The filtrate was concentrated to give the crude glycosylated product as a yellow syrup (15.4 g) which was dissolved in minimum DCM and the solution purified by column chromatography (4×22 cm of silica) using a gradient solvent of 25-35% EtOAc in hexane to give 2,3,4,6-tetra-O-benzoyl-β-D-glucopyranose-N-Boc-N-methylproploxyamine conjugate as a colourless syrup (7.95 g, 77%) (R_{f} 0.4 in 35% EtOAc in hexane).

¹H NMR (400 MHz, CDCl₃) δ 8.05-7.99 (m, 2H), 7.98-7.93 (m, 2H), 7.92-7.88 (m, 2H), 7.86-7.79 (m, 2H), 7.59-7.46 (m, 3H), 7.46-7.24 (m, 9H), 5.90 (dd, J=9.7, 9.7 Hz, 1H), 5.68 (dd, J=9.7, 9.7 Hz, 1H), 5.52 (dd, J=9.7, 7.8 Hz, 1H), 4.84 (d, J=7.8 Hz, 1H), 4.64 (dd, J=12.2, 3.2 Hz, 1H), 4.49 (dd, J=12.2, 5.2 Hz, 1H), 4.20-4.13 (m, 1H), 3-95 (ddd, J=9.7, 5.2, 3.2 Hz, 1H), 3.61-3.50 (m, 1H), 3.24-2.99 (m, 2H), 2.66 (s, 3H), 1.82-1.68 (m, 2H), 1.39 (s, 9H).

Step 2: To a 500 mL RBF containing 2,3,4,6-tetra-O-benzoyl-β-D-glucopyranose-N-Boc-N-methylproploxyamine conjugate (7.95 g, 10-35 mmol, 1 eq) was added DCM (50 mL) and a stirrer bar. The mixture was stirred (250 rpm) briefly until a solution had formed before TFA (10 mL) was added. The mixture was stirred for 0.5 hours and monitored by TLC (30% EtOAc/hexanes, R_{f} (starting material)=0.4, R_{f} (product)=0), visualised by UV). The reaction was concentrated by rotary evaporation and then reconcentrated from CHCl₃ (2×30 mL) to give 2,3,4,6-tetra-O-benzoyl-β-D-glucopyranose-N-methylproploxyammonium trifluoroacetate conjugate as lightly coloured syrup (11.0 g) that was used without further purification.

¹H NMR (400 MHz, CDCl₃) δ 8.69 (br s, 1H), 8.15 (br s, 1H), 8.08-8.02 (m, 2H), 7.99-7.88 (m, 4H), 7.87-7.80 (m, 2H), 7.67 (br s, 1H), 7.63-7.48 (m, 3H), 7.52-7.24 (m, 9H), 5.99 (dd, J=9.8, 9.8 Hz, 1H), 5.71 (dd, J=9.8, 9.8 Hz, 1H), 5.38 (dd, J=9.8, 7.8 Hz, 1H), 4.82 (d, J=7.8 Hz, 1H), 4.74 (dd, J=12.3, 2.8 Hz, 1H), 4.47 (dd, J=12.3, 5.0 Hz, 1H), 4.22-4.06 (m, 2H), 3.73 (app. p, J=5.1 Hz, 1H), 3.31-3.18 (m, 1H), 3.14-3.01 (m, 1H), 2.79 (t, J=5.3 Hz, 3H), 2.04 (app. p, J=5.4 Hz, 2H).

Step 3: A 500 mL RBF was charged with a stirrer bar, phyllochlorin (4.05 g, 7.96 mmol, 1 eq) and DCM (60 mL). 2,3,4,6-Tetra-O-benzoyl-β-D-glucopyranose-N-methylpropyloxyammonium trifluoroacetate conjugate (6.91 g, 10.35 mmol, 1.3 eq) was dissolved in DCM (20 mL) and transferred into the RBF. Triethylamine (6.62 mL, 47.8 mmol, 6 eq) was added, followed by PyBOP (4.97 g, 9.55 mmol, 1.2 eq), and the mixture stirred for 0.5 hours. TLC analysis showed consumption of the starting material and the presence of the product (5% MeOH/DCM, R_{f} (phyllochlorin)=0.25, R_{f} (product)=0.95, visualised by UV). The mixture was transferred to a separatory funnel and washed with 1M HCl (2×75 mL), then pH 7 phosphate buffer (100 mL), before being dried (Na₂SO₄) and concentrated by rotary evaporation to give the crude product as a green film (16.3 g). The crude product was purified by Biotage autocolumn chromatography (0-2% MeOH/DCM) to give phyllochlorin β-D-glucosyl-N-methylpropylamide conjugate tetrabenzoate as a green film (4.75 g, 52%) (HPLC purity: 95.9%).

Step 4: To a 500 mL RBF containing phyllochlorin β-D-glucosyl-N-methylpropylamide conjugate tetrabenzoate (4.65 g, 4.01 mmol, 1 eq) was added DCM (50 mL), MeOH (50 mL) and a stirrer bar. The mixture was stirred (300 rpm) briefly until a dark solution had formed, whereupon a solution of NaOMe (4.6M in MeOH, 0.44 mL, 2.01 mmol, 0.5 eq) was added, and the mixture stirred for 2 hours. TLC analysis at this point showed complete reaction (10% MeOH/DCM, R_{f} (starting material)=0.95, R_{f} (product)=0). The reaction mixture was concentrated and the residue purified by Biotage autocolumn chromatography (5-12% MeOH/DCM) to give compound 15 as a blue/green flaky solid (2.34 g, 79%) (HPLC purity: 99.4%). The ¹H NMR (400 MHz, CD₃OD) is shown in FIG. 10.

### Synthesis Example 16—Synthesis of phyllochlorin α-D-1-thiomannose-N-methylpropylamide conjugate (Compound 16)

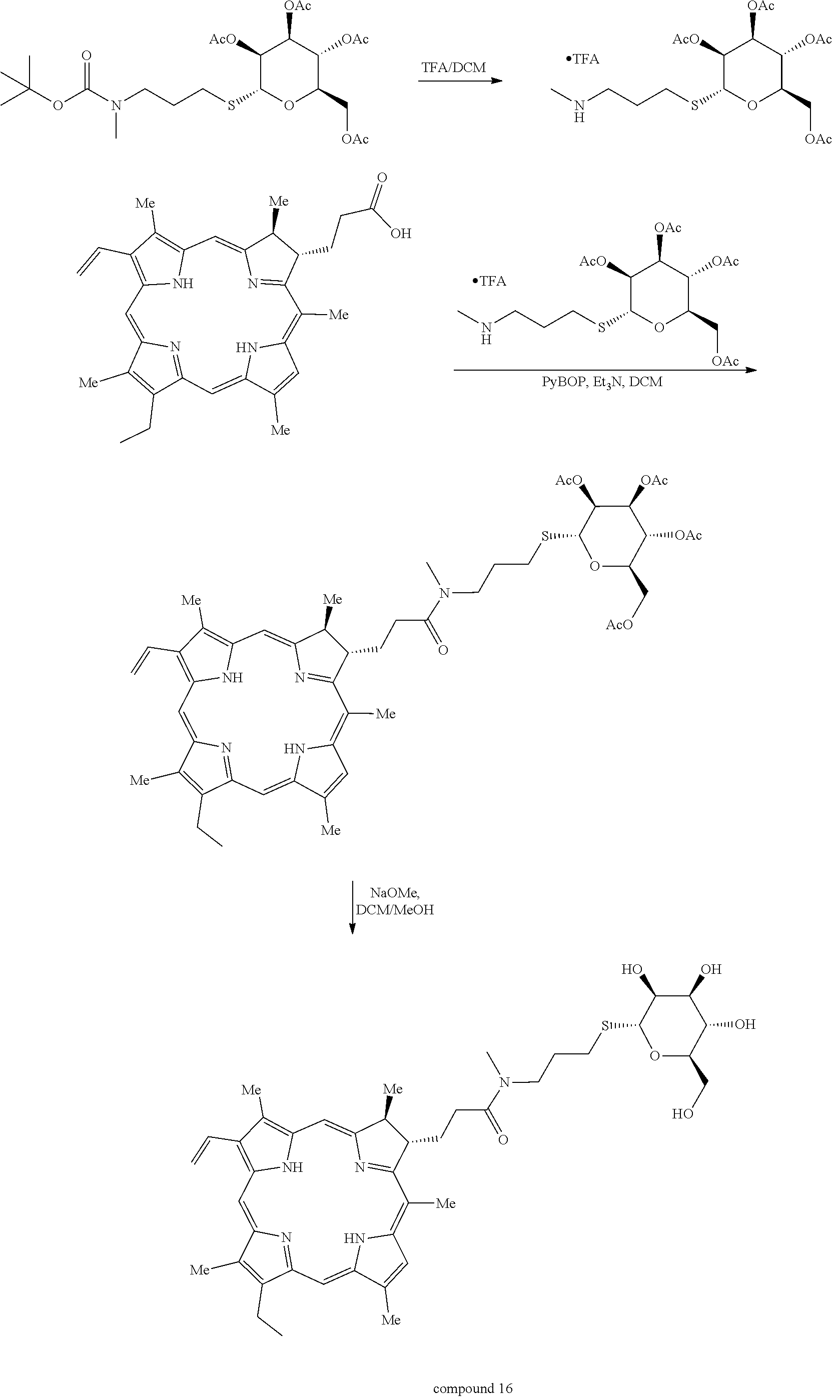

Step 1: To a solution of (2R,3R,4S,5S,6R)-2-(acetoxymethyl)-6-((3-((tert-butoxycarbonyl)(methyl)amino)propyl)thio)tetrahydro-2H-pyran-3,4,5-triyl triacetate (250 mg, 0.4668 mmol, 1.3 eq) in DCM (2.3 mL) was added TFA (0.6 mL). The resultant solution was stirred (420 rpm) for 1 hour at ambient temperature, and then concentrated by rotary evaporation. The residue was resuspended and concentrated twice from chloroform (2×3 mL) to give the deprotected amine as a viscous oil that was dissolved in DCM (1 mL) for the subsequent coupling reaction.

Step 2: To a 10 mL RBF was added phyllochlorin (182.6 mg, 0.3590 mmol, 1 eq), PyBOP (242.9 mg, 0.46678 mmol, 1.3 eq), DCM (2 mL) and triethylamine (0.39 mL, 2.5005 mmol, 6 eq). The resultant mixture was stirred (420 rpm) under nitrogen at ambient temperature for 15 minutes, and then the prepared solution of the deprotected amine in DCM was added in one portion. The resultant mixture was stirred for 90 minutes. The reaction mixture was transferred to a separatory funnel and washed with 1M HCl (2×3 mL), then pH 7 buffer (1×5 mL). The organic phase was dried (Na₂SO₄) and concentrated by rotary evaporation to give a bubbly blue/black film. The crude product was purified by Biotage autocolumn chromatography (0-5% MeOH/DCM) to afford phyllochlorin α-D-1-thiomannose-N-methylpropylamide conjugate peracetate as a blue/black solid (0.16 g, 48%).

Step 3: To a solution of phyllochlorin α-D-1-thiomannose-N-methylpropylamide conjugate peracetate (0.18 g, 0.1728 mmol, 1 eq) in MeOH (2 mL) and DCM (2 mL) was added NaOMe (4.6M in MeOH, 38 μL, 0.1728 mmol, 1 eq), and the mixture stirred (420 rpm) under N₂ for 90 minutes. The reaction was quenched with AcOH (10.0 μL, 0.1728 mmol, 1 eq) and concentrated by rotary evaporation to give a black film. The crude amide was purified by Biotage autocolumn chromatography to afford compound 16 as a blue/black solid (71.8 mg, 55%).

¹H NMR (400 MHz, d₆-DMSO) δ 9.75 (s, 1H), 9.74 (s, 1H), 9.10 (d, J=9.5 Hz, 1H), 9.06 (s, 1H), 8.34 (dd, J=17.8, 11.6 Hz, 1H), 6.44 (dd, J=17.7, 1.6 Hz, 1H), 6.17 (dd, J=11.6, 1.5 Hz, 1H), 5.15 (dd, J=40-4, 1.4 Hz, 1H), 5.09-4.37 (m, 5H), 3.98 (d, J=6.7 Hz, 3H), 3.82 (q, J=7.4 Hz, 2H), 3.77-3.64 (m, 2H), 3.64-3.59 (m, 4H), 3.54 (d, J=2.2 Hz, 3H), 3.34-3.24 (m, 4H), 2.96-2.91 (m, 1H), 2.88 (s, 2H), 2.80 (s, 1H), 2.65-2.53 (m, 1H), 2.48-2.33 (m, 1H), 1.90-1.76 (m, 2H), 1.69 (t, J=7.6 Hz, 7H), −2.27 (s, 1H), −2.43 (s, 1H).

### Synthesis Example 17—Synthesis of phyllochlorin β-D-1-thiomannose-N-methylpropylamide conjugate (Compound 17)

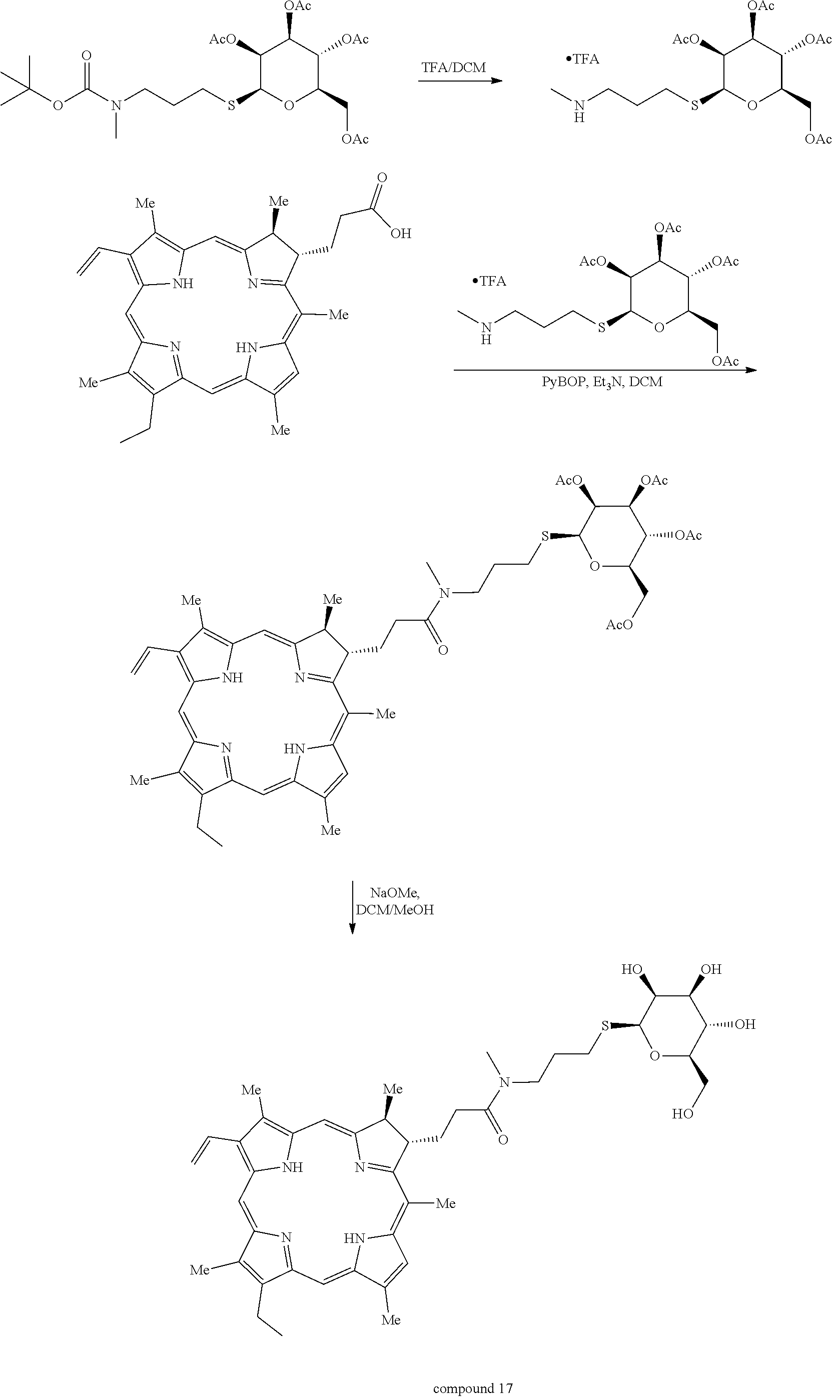

Step 1: To a solution of (2R,3R,4S,5S,6S)-2-(acetoxymethyl)-6-((3-((tert-butoxycarbonyl)(methyl)amino)propyl)thio)tetrahydro-2H-pyran-3,4,5-triyl triacetate (250 mg, 0.4668 mmol, 1.3 eq) in DCM (2.3 mL) was added TFA (0.6 mL). The resultant solution was stirred (420 rpm) for 1 hour at ambient temperature, and then concentrated by rotary evaporation. The residue was resuspended and concentrated twice from chloroform (2×3 mL) to give the deprotected amine as a viscous oil that was dissolved in DCM (1 mL) for the subsequent coupling reaction.

Step 2: To a 10 mL RBF was added phyllochlorin (182.6 mg, 0.3590 mmol, 1 eq), PyBOP (242.9 mg, 0.46678 mmol, 1.3 eq), DCM (2 mL) and triethylamine (0.39 mL, 2.5005 mmol, 6 eq). The resultant mixture was stirred (420 rpm) under nitrogen at ambient temperature for 15 minutes, and then the prepared solution of the deprotected amine in DCM was added in one portion. The resultant mixture was stirred for 90 minutes. The reaction mixture was transferred to a separatory funnel and washed with 1M HCl (2×3 mL), then pH 7 buffer (1×5 mL). The organic phase was dried (Na₂SO₄) and concentrated by rotary evaporation to give a bubbly blue/black film. The crude product was purified by Biotage autocolumn chromatography (0-5% MeOH/DCM) to afford phyllochlorin β-D-1-thiomannose-N-methylpropylamide conjugate peracetate as a blue/black solid (0.18 g, 54%).

Step 3: To a solution of phyllochlorin β-D-1-thiomannose-N-methylpropylamide conjugate peracetate (0.18 g, 0.1944 mmol, 1 eq) in MeOH (2 mL) and DCM (2 mL) was added NaOMe (4.6M in MeOH, 43 μL, 0.1944 mmol, 1 eq), and the mixture stirred (420 rpm) under N₂ for 90 minutes. The reaction was quenched with AcOH (11.1 μL, 0.1944 mmol, 1 eq) and concentrated by rotary evaporation to give a black film. The crude amide was purified by Biotage autocolumn chromatography to afford compound 17 as a blue/black solid (107.9 mg, 73%).

¹H NMR (400 MHz, d₆-DMSO) δ 9.74 (s, 1H), 9.73 (s, 1H), 9.11 (d, J=16.9 Hz, 1H), 9.06 (s, 1H), 8.33 (dd, J=17.8, 11.6 Hz, 1H), 6.43 (dd, J=17.9, 1.6 Hz, 1H), 6.16 (dd, J=11.6, 1.5 Hz, 1H), 4.88-4.52 (m, 7H), 4.47 (dt, J=22.0, 5.8 Hz, 1H), 3.98 (d, J=5.7 Hz, 3H), 3.80 (q, J=7.8 Hz, 2H), 3.77-3.71 (m, 1H), 3.64-3.58 (m, 3H), 3.54 (d, J=3.8 Hz, 3H), 3.32 (s, 5H), 2.89 (s, 2H), 2.78 (s, 1H), 2.55 (q, J=7.6 Hz, 2H), 2.46-2.35 (m, 1H), 1.89-1.75 (m, 1H), 1.75-1.64 (m, 7H), −2.26 (s, 1H), −2.42 (d, J=2.1 Hz, 1H).

### Synthesis Example 18—Synthesis of 3-((7S,8S)-18-ethyl-2,5,8,12,17-pentamethyl-13-vinyl-7H,8H-porphyrin-7-yl)-N-(2-(2-hydroxyethoxy)ethyl)-N-methylpropanamide (also called phyllochlorin N-(2-(2-hydroxyethoxy)ethyl)-N-methylpropylamide) (Compound 18)

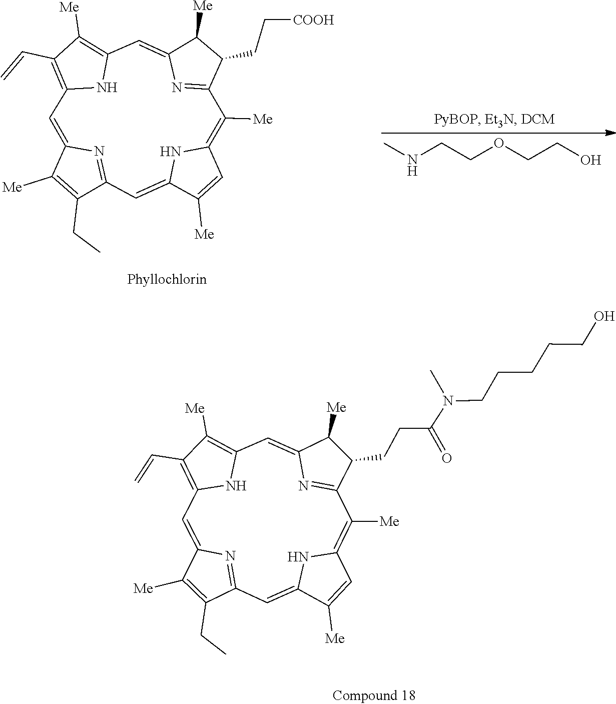

Into a 100 mL RBF fitted with a nitrogen inlet and containing a stirrer bar was added phyllochlorin (2.00 g, 3.93 mmol, 1 eq), dichloromethane (40 mL), PyBOP (2.46 g, 4.72 mmol, 1.2 eq) and triethylamine (0.87 mL, 3 eq). The mixture was stirred at room temperature for 30 minutes and then 2-(2-methylaminoethoxy)ethanol (0.61 g, 1.3 eq) in DCM (5 mL) was added in one portion. The mixture was stirred at room temperature overnight. Analysis by HPLC showed the reaction to be complete. The reaction mixture was transferred to a separatory funnel and washed with 1M HCl (2×50 mL), then pH 7 buffer (2×50 mL). The organic layer was dried (Na₂SO₄) and concentrated by rotary evaporation to give the crude product as a blue/green film (4.2 g) which was purified by column chromatography (5×25 cm of silica) eluting with 1-3% MeOH/DCM. Pure fractions by TLC(R_{f} 0.40 in 5% MeOH/DCM) were combined to give compound 18 (1.40 g, 58%) (HPLC purity: 94.4%).

¹H NMR (400 MHz, CDCl₃) δ 9.72 (m, 2H), 8.87-8.82 (m, 1H), 8.16 (dd, 1H), 6.39 (dd, 1H), 6.15 (dd, 1H), 4.72-4.65 (m, 1H), 4.59-4.50 (m, 1H), 4.01 (s, 3H), 3.85 (q, 2H), 3.65 (s, 3H), 3.60-3.55 (m, 2H), 3.53 (s, 3H), 3.41-3.38 (m, 2H), 3.37 (s, 3H), 3.35-3.30 (m, 2H), 2.76-2.71 (m, 1H), 2.65-2.50 (m, 4H), 2.50-2.40 (m, 1H), 2.38-2.30 (m, 3H), 2.30-2.20 (m, 2H), 1.90-1.80 (m, 1H), 1.80-1.73 (m, 7H), −2.09 (br s, 1H), −2.22 (br s, 1H).

### Synthesis Example 19—Synthesis of 3-((7S,8S)-18-ethyl-2,5,8,12,17-pentamethyl-13-vinyl-7H,8H-porphyrin-7-yl)-N-methyl-N-(3-(((2S,3R,4S,5S,6R)-3,4,5-trihydroxy-6-(hydroxymethyl)tetrahydro-2H-pyran-2-yl)thio)propyl)propanamide zinc complex (Compound 19)

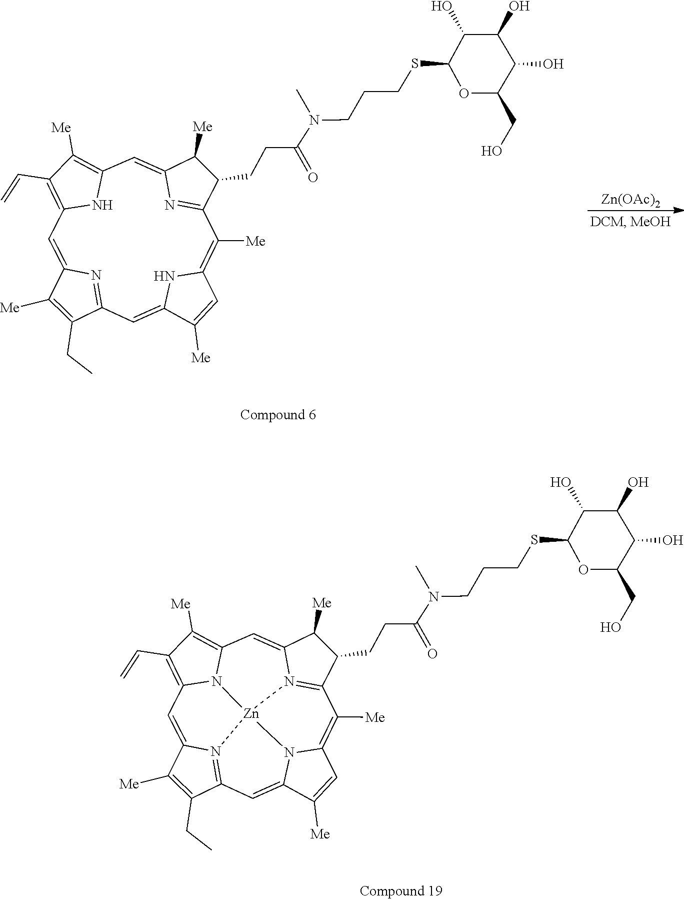

To a 25 mL single-neck RBF was added phyllochlorin β-D-1-thioglucose-N-methylpropylamide conjugate (50 mg, 0.066 mmol, 1 eq) and DCM (2 mL). A solution of zinc acetate (24 mg, 0.132 mmol, 2 eq) in methanol (1 mL) was added and the mixture was stirred under nitrogen in the dark for 2 hours. The solution was then diluted with DCM (20 mL) and washed with water (3×25 mL), dried (Na₂SO₄) and concentrated to give compound 19 as a blue solid (54 mg, quantitative) (HPLC purity: 98.5%).

¹H NMR (400 MHz, CDCl₃) δ 9.80-9.20 (br s, 2H), 8.70-8.50 (br s, 2H), 8.20-8.00 (br s, 1H), 6.25-5.90 (br m, 2H), 4.50-4.00 (br s, 2H), 4.00-3.20 (br m, 14H), 3.10 (br m, 2H), 2.70-2.10 (br m, 10H), 2.00-1.30 (br m, 28H), 1.20-0.80 (br m, 5H).

### Synthesis Example 20—Synthesis of 3-((7S,8S)-18-ethyl-2,5,8,12,17-pentamethyl-13-vinyl-7H,8H-porphyrin-7-yl)-N-(2-(2-(((2R,3R,4S,5S,6R)-3,4,5-trihydroxy-6-(hydroxymethyl)tetrahydro-2H-pyran-2-yl)oxy)ethoxy)ethyl)propanamide (Compound 20)

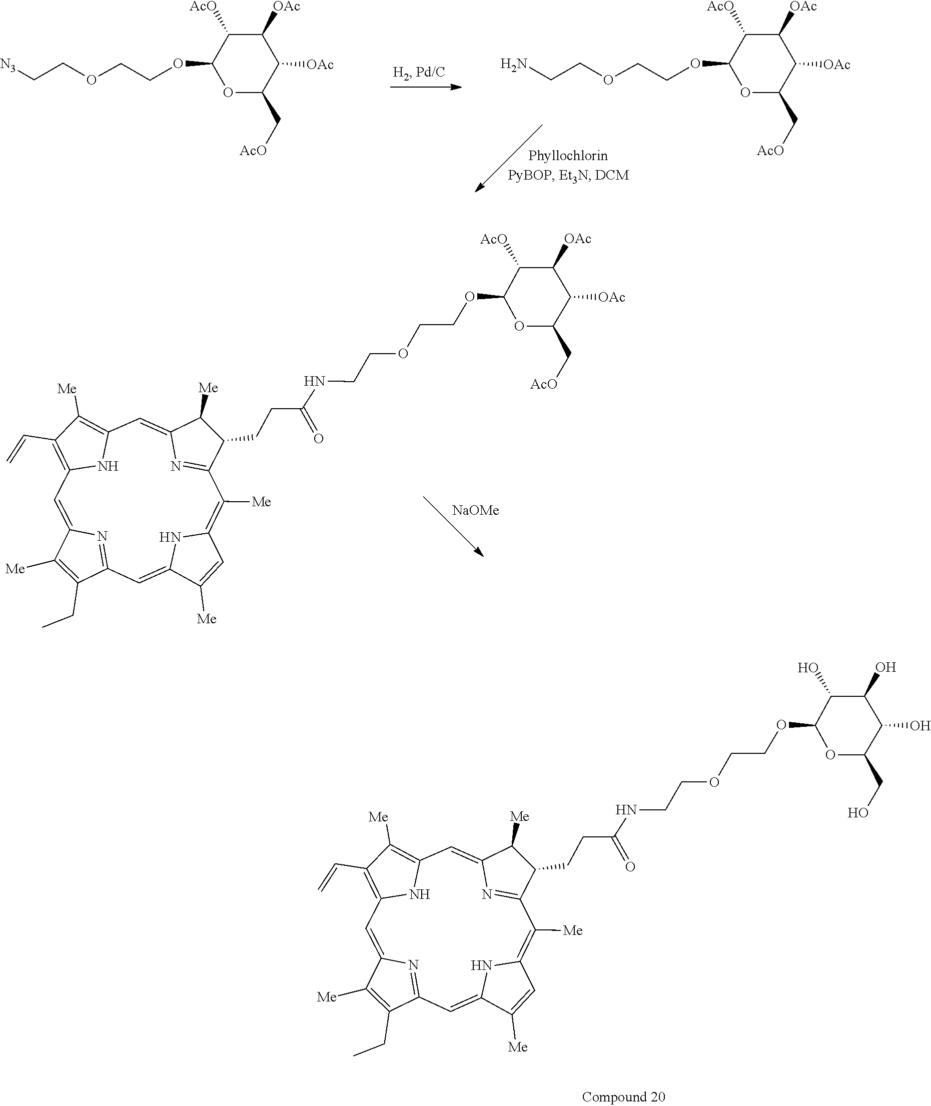 [SMILES: CC(=O)OC[C@H]1O[C@@H](OCCOCCN=[N+]=[N-])[C@H](OC(C)=O)[C@@H](OC(C)=O)[C@@H]1OC(C)=O ; CC(=O)OC[C@H]1O[C@@H](OCCOCCN)[C@H](OC(C)=O)[C@@H](OC(C)=O)[C@@H]1OC(C)=O ; C=Cc1c(C)c2cc3nc(c(C)c4cc(C)c(cc5nc(cc1[nH]2)C(C)=C5CC)[nH]4)[C@@H](CCC(=O)NCCOCCO[C@@H]1O[C@H](COC(C)=O)[C@@H](OC(C)=O)[C@H](OC(C)=O)[C@H]1OC(C)=O)[C@@H]3C ; C=Cc1c(C)c2cc3nc(c(C)c4cc(C)c(cc5nc(cc1[nH]2)C(C)=C5CC)[nH]4)[C@@H](CCC(=O)NCCOCCO[C@@H]1O[C@H](CO)[C@@H](O)[C@H](O)[C@H]1O)[C@@H]3C ; "NaOMe"]

Step 1: A 3-neck 100 mL RBF was charged with (2R,3R,4S,5R,6R)-2-(acetoxymethyl)-6-(2-(2-azidoethoxy)ethoxy)tetrahydro-2H-pyran-3,4,5-triyl triacetate (1.00 g, 2.167 mmol, 1 eq), 10% Pd/C (25 mg), methanol (100 mL) and a stirrer bar. A hydrogen balloon was connected and the flask was evacuated and then re-filled with nitrogen (3 times), evacuated and re-filled with hydrogen (2 times). The resulting solution was then stirred (550 rpm) under the hydrogen atmosphere for 1 hour. The solution was filtered through Celite® (0.5×3 cm), washing with DCM (˜30 mL) and the solvent then so removed under reduced pressure to give 1.02 g of crude amine product that was used directly in the next step.

Step 2: To a 50 mL RBF was added phyllochlorin (0.85 g, 1.67 mmol, 1 eq), PyBOP (1.04 g, 2.01 mmol, 1.2 eq), DCM (20 mL) and triethylamine (1.39 mL, 10.03 mmol, 6 eq). The resultant mixture was stirred (250 rpm) under nitrogen at ambient temperature for 15 minutes, and then the amine (1.02 g) in DCM (5 mL) was added in one portion. The resultant mixture was stirred overnight in the dark under nitrogen. The reaction mixture was diluted with DCM (30 mL), transferred to a separatory funnel and washed with 1M HCl (2×75 mL), then pH 7 buffer (1×100 mL). The organic phase was dried (Na₂SO₄) and concentrated by rotary evaporation to give a blue/black oil (˜2.5 g) which was purified by column chromatography (4×25 cm of silica) eluting with 1-2.5% MeOH/DCM. Fractions containing the product (R_{f}˜0.4 in 5% MeOH/DCM) were combined to give phyllochlorin β-D-1-glucose-N-ethoxyethyl amide conjugate tetraacetate as a blue/black solid (0.65 g, 42%) (HPLC purity: 83%) which was deprotected without further purification.

Step 3: To a solution of phyllochlorin β-D-1-glucose-N-ethoxyethyl amide conjugate tetraacetate (600 mg, 0.648 mmol, 1 eq) in MeOH (7 mL) and DCM (7 mL) was added NaOMe (4.6M in MeOH, 0.14 mL, 0.648 mmol, 1 eq), and the mixture stirred (300 rpm) under nitrogen for 90 minutes. HPLC analysis showed conversion to the deacetylated product. The reaction was quenched with AcOH (19 mg, 0.5 eq) and concentrated by rotary evaporation to give a black film which was purified by column chromatography (4×24 cm of silica) eluting with 5-12% MeOH/DCM. Fractions having R_{f} 0.20 in 10% MeOH/DCM were combined to give compound 20 as a blue/black solid (310 mg, 63%).

### Synthesis Example 21—Synthesis of 3-((7S,8S)-18-ethyl-2,5,8,12,17-pentamethyl-13-vinyl-7H,8H-porphyrin-7-yl)-N-(2-(2-hydroxyethoxy)ethyl)-N-methylpropanamide zinc complex (Compound 21)

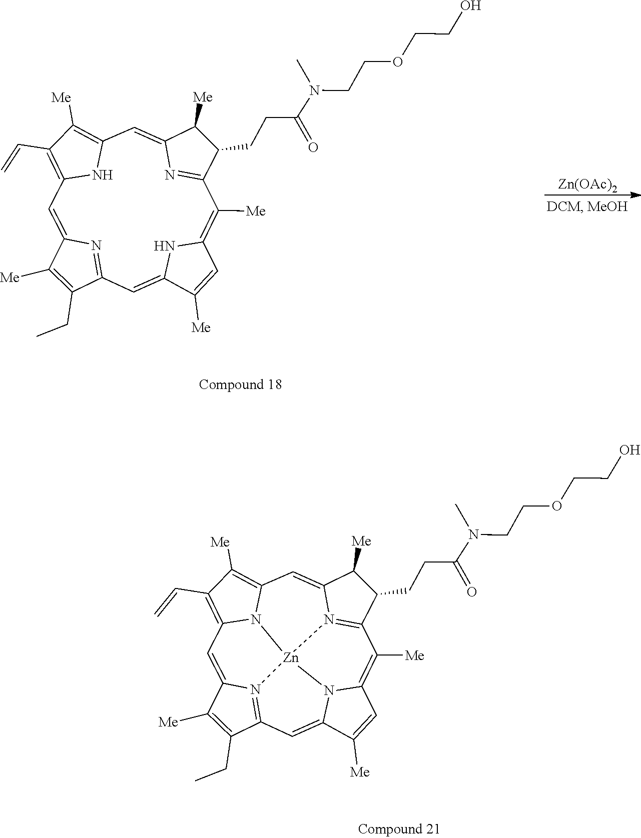

To a 50 mL single-neck RBF was added phyllochlorin N-(2-(2-hydroxyethoxy)ethyl)-N-methylpropylamide (200 mg, 0.328 mmol, 1 eq) and DCM (10 mL). A solution of zinc acetate (120 mg, 0.656 mmol, 2 eq) in methanol (5 mL) was added and the mixture was stirred under nitrogen in the dark for 2 hours. The solution was then diluted with DCM (20 mL) and washed with water (3×25 mL), dried (Na₂SO₄) and concentrated to give crude product as a blue solid. The crude product was purified by column chromatography (3×18 cm of silica) using a 3% MeOH/DCM solvent mixture. The pure fractions (R_{f}=0.45 in 5% MeOH/DCM) were combined to give compound 21 as a blue flaky solid (203 mg, 92%) (HPLC purity: 93.2%).

¹H NMR (400 MHz, CDCl₃) δ 9.80-9.25 (br s, 2H), 8.80-8.50 (br s, 2H), 8.20-8.00 (br s, 1H), 6.30-6.15 (br m, 1H), 6.10-5.95 (br m, 1H), 4.80-4.10 (br m, 2H), 4.00-3.70 (br m, 9H), 3.70-3.20 (br m, 14H), 2.85-2.60 (br m, 4H), 2.50 (br s, 3H), 2.50-2.30 (br m, 5H), 2.25-2.20 (m, 5H), 1.85-1.60 (m, 12H), 1.50-1.40 (m, 5H), 1.20-1.00 (br m, 2H).

### Synthesis Example 22—Synthesis of phyllochlorin (6-hexyl)triphenylphosphonium chloride propylamide (Compound 22)

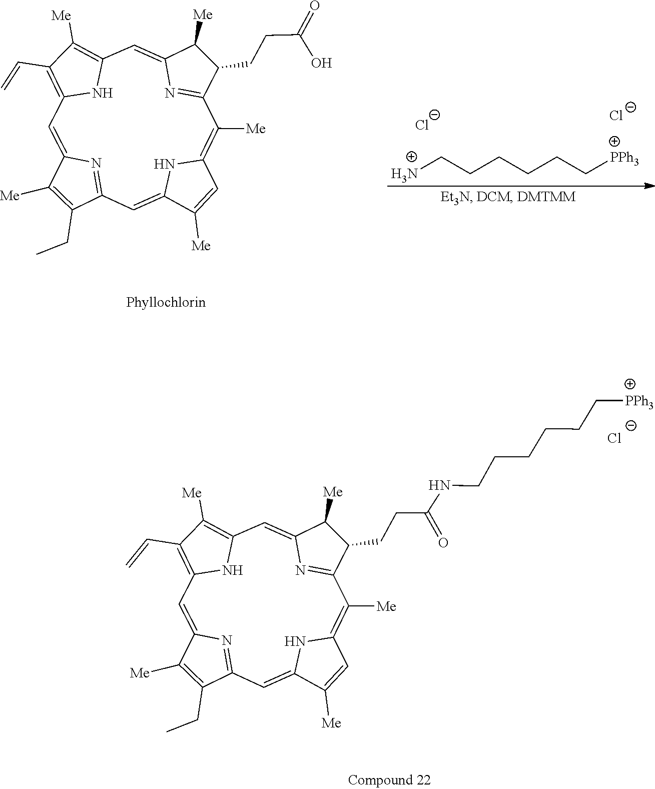

To a 25 mL RBF was added phyllochlorin (100 mg, 0.1966 mmol, 1 eq), 4-(4,6-dimethoxy-1,3,5-triazin-2-yl)-4-methylmorpholin-4-ium chloride (DMTMM) (76 mg, 0.2752 mmol, 1.4 eq), DCM (5 mL), triethylamine (44 μL, 0.3145 mmol, 1.6 eq) and (6-aminohexyl)triphenylphosphonium chloride hydrochloride (120 mg, 0.2752 mmol, 1.4 eq). The resultant mixture was stirred (420 rpm) under nitrogen at ambient temperature for 30 minutes. The reaction mixture was concentrated using a rotary evaporator and the crude residue purified by column chromatography (silica, 5-7% MeOH/DCM) to give compound 22 as a dark blue/green solid (180 mg, quantitative).

¹H NMR (400 MHz, CDCl₃) δ 9.67 (m, 2H), 8.89 (s, 1H), 8.78 (s, 1H), 8.14 (dd, 1H), 7.83 (t, 1H), 7.62 (m, 6H), 7.48 (m, 9H), 6.35 (d, 1H), 6.10 (d, 1H), 4.70 (q, 1H), 4.51 (m, 1H), 3.98 (m, 7H), 3.82 (q, 2H), 3.60 (s, 3H), 3.49 (m, 5H), 3.35 (s, 3H), 3.26 (m, 2H), 2.88 (m, 1H), 2.61-2.48 (m, 2H), 1.90-1.80 (m, 1H), 1.78-1.70 (m, 6H), 1.60-1.40 (m, 8H), −2.11 (br s, 1H), −2.24 (br s, 1H).

### Synthesis Example 23—Synthesis of phyllochlorin N-meglumine-propylamide (Compound 23)

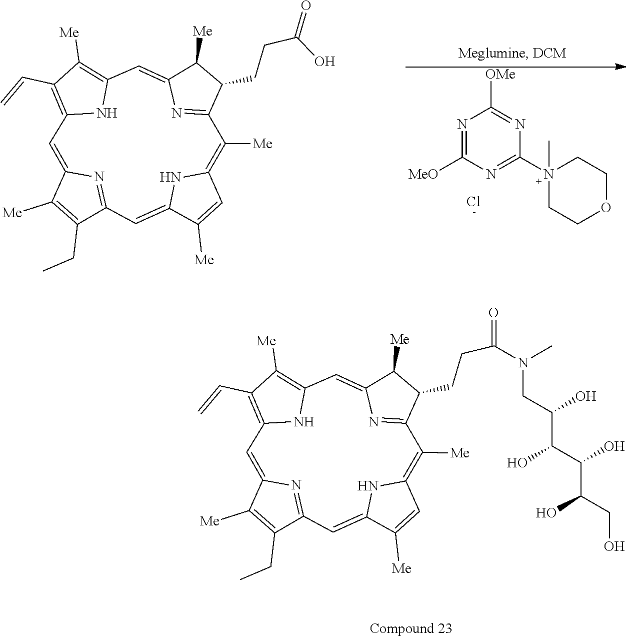

To a 50 mL RBF was added phyllochlorin (0.50 g, 0.983 mmol, 1 eq), DMTMM (0.30 g, 1.081 mmol, 1.1 eq), DCM (15 mL) and meglumine (0.23 g, 1-179 mmol, 1.2 eq). The resultant mixture was stirred under nitrogen at ambient temperature for 1 hour. A further portion of meglumine (0.10 g, 0.51 mmol, 0.5 eq) was added and the solution so stirred for a further 3 hours. The reaction mixture was transferred to a separatory funnel, diluted with chloroform (30 mL) and washed with 0.5M HCl (50 mL). The aqueous layer was re-extracted with chloroform and the combined organics washed with pH 7 buffer. The organic phase was dried (Na₂SO₄) and concentrated by rotary evaporation to give a blue/black film, which was purified by column chromatography (silica) using a gradient of 5% MeOH/DCM (50 mL), then 7% MeOH/DCM (100 mL), then 9% MeOH/DCM (100 mL), and then 10% MeOH/DCM (200 mL) to give compound 23 as a dark blue/green solid (432 mg, 64%).

¹H NMR (400 MHz, d₆-DMSO) δ 9.74 (s, 1H), 9.72 (s, 1H), 9.11 (s, 1H), 9.07 (s, 1H), 8.34 (dd, 1H), 6.44 (d, 1H), 6.17 (d, 1H), 5.10 & 4.75 (2×d, 1H), 4.65-4.40 (m, 5H), 4.30 (m, 1H), 4.00 (m, 3H), 3.90 (m, 1H), 3.80 (m, 2H), 3.68 (m, 1H), 3.62 (s, 3H), 3.60-3.50 (m, 5H), 3.50-3.40 (m, 2H), 3.30-3.08 (m, 1H), 3.00 (s, 1H), 2.90 (s, 2H), 2.86-2.60 (m, 1H), 2.45-2.35 (m, 1H), 1.75-1.65 (m, 6H), 1.75-1.50 (m, 1H), −2.26 (s, 1H), −2.42 (s, 1H).

### Synthesis Example 24—Synthesis of phyllochlorin 2-deoxy-D-mannosamine-propylamide (Compound 24)

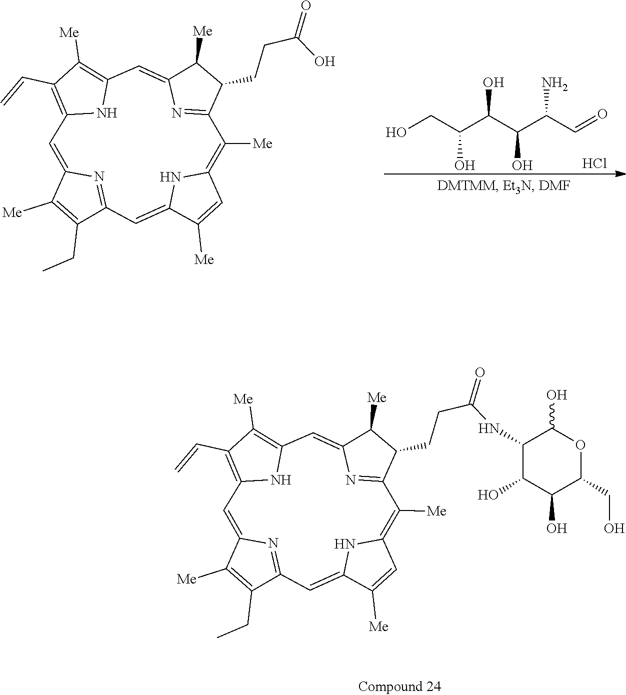 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; N[C@H](C=O)[C@@H](O)[C@H](O)[C@H](O)CO ; Cl ; C=Cc1c(C)c2cc3nc(c(C)c4cc(C)c(cc5nc(cc1[nH]2)C(C)=C5CC)[nH]4)[C@@H](CCC(=O)N[C@@H]1C(O)O[C@H](CO)[C@@H](O)[C@@H]1O)[C@@H]3C]

To a 25 mL RBF containing a stirrer bar was added phyllochlorin (250 mg, 0.491 mmol, 1 eq), DMTMM (127 mg, 0.541 mmol, 1.1 eq), DMF (3 mL), triethylamine (75 μL, 0.541 mmol, 1.1 eq) and (D)-mannosamine hydrochloride (117 mg, 0.541 mmol, 1.1 eq). The resultant mixture was stirred for 30 minutes. Further DMTMM (13 mg, 0.1 eq), triethylamine (7 μL, 0.1 eq) and (D)-mannosamine hydrochloride (10 mg, 0.1 eq) were added and the solution stirred for a further 30 minutes. The reaction mixture was concentrated by rotary evaporation to give a black residue. The residue was taken up in DCM (20 mL) and washed with 0.5M HCl (10 mL). The DCM layer was collected and washed with pH 7 phosphate buffer. The mixture was extracted with 1:1 DCM/MeOH (3×10 mL), dried (Na₂SO₄) and concentrated to give a dark solid (˜220 mg), which was subjected to column chromatography (silica). The column was eluted using a gradient of 8% MeOH/DCM, then 12% MeOH/DCM, and then 14% MeOH/DCM. Fractions containing compound 24 were combined and dried under vacuum to give a dark blue solid (105 mg, 32%).

¹H NMR (400 MHz, d₆-DMSO) δ 9.76 (s, 1H), 9.74 (s, 1H), 9.05-9.12 (m, 2H), 8.34 (dd, 1H), 7.54 & 7.18 (2×d, 1H), 6.60-6.42 (m, 2H), 6.17 (d, 1H), 4.93 (m, 1H), 4.72-4.50 (m, 4H), 4.30-4.20 (m, 1H), 4.01 (m, 1H), 3.95 (m, 3H), 3.82 (q, 2H), 3.80-3.72 (m, 1H), 3.65-3.60 (m, 4H), 3.58-3.52 (m, 4H), 3.50-3.40 (m, 2H), 3.17-3.02 (m, 1H), 2.68-2.57 (m, 1H), 2.48-2.38 (m, 1H), 2.25-2.12 (m, 1H), 1.82-1.67 (m, 7H), −2.26 (brs, 1H), −2.43 (brs, 1H). The product exists as a mixture of epimers which causes two sets of signals in an ˜3:1 ratio.

### Synthesis Example 25—Synthesis of phyllochlorin 2-deoxy-D-galactosamine-propylamide (Compound 25)

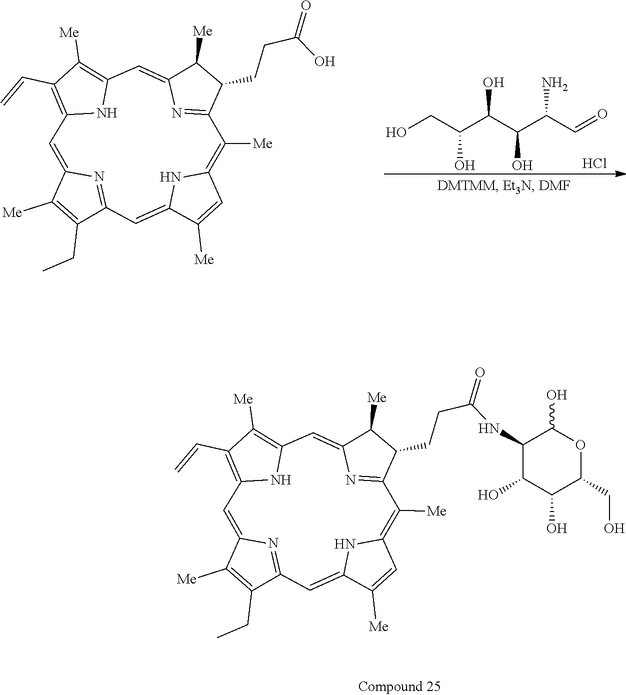 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; N[C@H](C=O)[C@@H](O)[C@H](O)[C@H](O)CO ; Cl ; C=Cc1c(C)c2cc3nc(c(C)c4cc(C)c(cc5nc(cc1[nH]2)C(C)=C5CC)[nH]4)[C@@H](CCC(=O)N[C@H]1C(O)O[C@H](CO)[C@H](O)[C@@H]1O)[C@@H]3C]

To a 25 mL RBF containing a stirrer bar was added phyllochlorin (250 mg, 0.491 mmol, 1 eq), DMTMM (149 mg, 0.541 mmol, 1.1 eq), DMF (3 mL), triethylamine (75 μL, 0.541 mmol, 1.1 eq) and D-(+)-galactosamine hydrochloride (116 mg, 0.541 mmol, 1.1 eq). The resultant mixture was stirred for 30 minutes, at which point HPLC analysis showed ˜4% phyllochlorin remained. Further DMTMM (14 mg, 0.1 eq), triethylamine (7 μL, 0.1 eq) and D-(+)-galactosamine hydrochloride (11 mg, 0.1 eq) were added and the solution stirred for a further 30 minutes. The reaction mixture was concentrated by rotary so evaporation to give a black residue. The residue was subjected to column chromatography. The column was eluted using a gradient of 10% MeOH/DCM (300 mL), then 15% MeOH/DCM (300 mL), and then 20% MeOH/DCM. Fractions containing the product were combined and the solvent removed by rotary evaporation. The solid was dried under vacuum at 60° C. to give compound 25 as a dark blue solid (275 mg, 84%).

¹H NMR (400 MHz, d₆-DMSO) δ 9.74 (d, J=6.2 Hz, 2H), 9.13-8.95 (m, 2H), 8.33 (dd, J=17.8, 11.6 Hz, 1H), 7.66 (dd, J=36.3, 8.5 Hz, 1H), 6.50-6.33 (m, 1H), 6.27-6.07 (m, 2H), 4.92 (t, J=3.9 Hz, 1H), 4.72-4.22 (m, 5H), 4.00-3.90 (m, 3H), 3.88-3.67 (m, 4H), 3.62 (d, J=0.9 Hz, 4H), 3.55 (s, 4H), 3.33 (s, 24H), 2.53-2.47 (m, 4H), 2.20-1.99 (m, 1H), 1.84-1.62 (m, 8H), −2.25 (s, 1H), −2.41 (s, 1H). The product exists as a mixture of epimers which causes two sets of signals in an ˜3:1 ratio.

### Biological Experimental Details

### Example 1—Determination of Solubility of Phyllochlorin Analogues

Absorbance maxima (660±2 nm for phyllochlorin) were used as a surrogate measure of solubility. The relevant phyllochlorin analogue was diluted to 50 μM in PBS (phosphate buffered saline) solutions containing decreasing amounts of DMSO from 100% to 0%.

As PBS buffer (pH 7.4) is used in the experiments and the carboxylic acid in phyllochlorin (and the same carboxy group in other related chlorins) is estimated to have a pKa of less than 5, phyllochlorin in the PBS buffer will exist predominantly as the sodium and potassium salts with very little to no free acid.

Where required, polyvinylpyrrolidone (K30) was added to a final concentration of 1% w/v. Absorbance was measured using a Cytation 3 Multimode Plate Reader (Biotek) in spectral scanning mode, with spectra captured between 500-800 nm in 2 nm increments. Equivalent blank solutions were also measured and subtracted accordingly. Each spectrum was normalized to have a minimum signal of 0, and a maximum signal in pure DMSO solution (the most soluble state) of 100%.

Results—Solubility and Absorbance Maxima of Phyllochlorin

The solubility of phyllochlorin was assessed in solutions containing DMSO as an organic solvent. Phyllochlorin (50 μM final concentration) was resuspended in 100% DMSO to fully dissolve the phyllochlorin. With phyllochlorin concentrations maintained at 50 μM the % DMSO was decreased in a stepwise manner from 100% to 0%. All solutions were mixed by vortexing, then centrifuged for 2 mins to pellet any insoluble material. A 150 μl aliquot was transferred to individual wells of a 96-well clear microplate, and absorbance spectra collected between 500-800 nm in 2 nm increments. Equivalent solutions containing decreasing % DMSO were used to control for background.

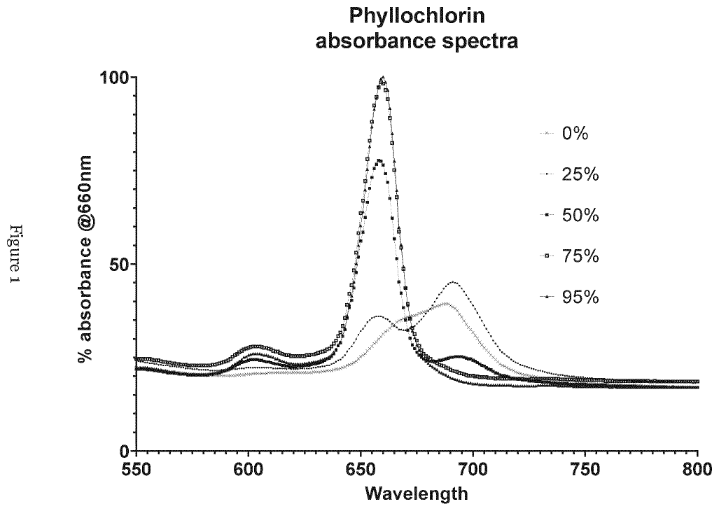

In aqueous solvent phyllochlorin displayed a single broad absorbance peak with maxima at 688±2 nm, which resolved into 2 distinct peaks with maxima at 688 and 660±2 nm, respectively (FIG. 1). The absorbance peak at 688 nm reduced with increasing DMSO concentration and at 75% DMSO only a single absorbance maxima at 660±2 nm was observed. Thus, the solubility of phyllochlorin was dependent on organic solvent concentration; and when fully solubilized in organic solvent, the absorbance maxima was found at 660±2 nm.

Results—Solubility of Phyllochlorin is Improved by Addition of PVP

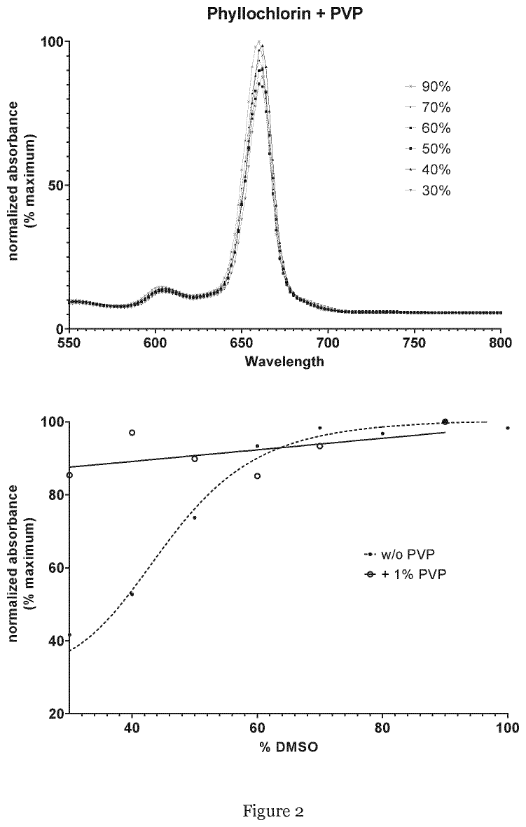

To improve solubility of phyllochlorin, the addition of PVP at 1% w/v was tested. PVP increased solubility substantially, and phyllochlorin remained soluble in aqueous solvent (without DMSO) when in the presence of 1% PVP. Moreover, the phyllochlorin absorbance peak at 688 nm was absent when PVP was introduced demonstrating the improved solubility of phyllochlorin in the presence of PVP (FIG. 2).

Results—Solubility and Absorbance Maxima of Phyllochlorin Analogues

The absorbance spectra of several phyllochlorin analogues (compounds 1-3) were so measured in the presence of 1% PVP (w/v) as previously described. The majority of phyllochlorin analogues tested had absorbance maxima at 660±2 nm, as previously determined in the presence of 1% PVP (w/v).

### Example 2—Cytotoxicity, Phototoxicity and Therapeutic Index

Preparation of Chlorin Stock Solutions

The photosensitizer (e.g. phyllochlorin, phyllochlorin analogue, chlorin e4 disodium (provided by Advanced Molecular Technologies, Scoresby) or Talaporfin sodium (purchased from Focus Bioscience cat #HY-16477-5MG)) was resuspended in 100% dimethylsulfoxide (DMSO) at a concentration of 5.5 mM. The samples were stored at 4° C. protected from light.

Cell Culture

Human ovarian cancer cell line SKOV3 (ATCC #HTB-77) was maintained in Dulbecco's Modified Eagle Medium/Nutrient Mixture F-12 (DMEM/F-12), supplemented with 10% v/v Fetal Bovine Serum, 100 U/mL penicillin and 100 g/mL streptomycin. Monolayer cultures were grown in a humidified incubator at 37° C. with 5% CO₂.

Chlorin Preparations for In Vitro Studies

For in vitro experiments, photosensitizers (stock solution 5.5 mM in 100% DMSO) were diluted in cell culture media (Dulbecco's Modified Eagle Medium/Nutrient Mixture F-12) supplemented with 10% v/v Fetal Bovine Serum, 100 U/mL penicillin, 100 g/mL streptomycin and 10% w/v Kollidon-12. Once cells had reached ˜80% confluence, spent media was replaced with media containing photosensitizer and cells were incubated for the desired period of time to allow photosensitizer uptake.

Statistical Analyses

All data were analysed using GraphPad PRISM v 8.3.1 (549) (GraphPad Software, CA). Spectral absorbance and viability measurements were normalized in the range 0-100%, with a minimum of 0 and a maximum value determined from the dataset. Dose response was determined using a sigmoidal four-point non-linear regression with variable slope, and IC50s calculated for each compound. All data are shown as mean±SD (where appropriate).

Cytotoxicity

SKOV3 cells were seeded in 96-well black wall plates (Greiner #655090) at a cell density of 5000 cells in 100 μl culture medium per well. On reaching ˜60% confluence, media was aspirated and replaced with fresh media containing the relevant phyllochlorin analogue from 0-100 μM in DMSO. Cells were incubated for a further 24 hours, allowing uptake of phyllochlorin analogues.

To test for inherent cytotoxicity (i.e. “dark toxicity”) of the phyllochlorin analogue the culture media was replaced after 24 hours with fresh media containing 10% (v/v) AlamarBlue Cell Viability Reagent (ThermoFisher) and cells incubated at 37° C. for 6 hours. Untreated cells were used as a control. Fluorescence (Ex 555 nm/Em 596 nm) was measured using a Cytation 3 Multimode Plate Reader (Biotek), and cytotoxicity assessed according to the % viable cells remaining. All measurements were made in quadruplicate.

Results—Cytotoxicity of Phyllochlorin Compared to Chlorin e4 Disodium

To assess the inherent cytotoxicity (i.e. “dark toxicity”) of phyllochlorin, SKOV3 ovarian cancer cells were incubated with concentrations of phyllochlorin from 0-100 μM (in DMSO) for a period of 24 hours. Cell viability was assessed using AlamarBlue Cell Viability Reagent, and compared to an untreated control. Measurements were made using phyllochlorin in both the presence or absence of PVP.

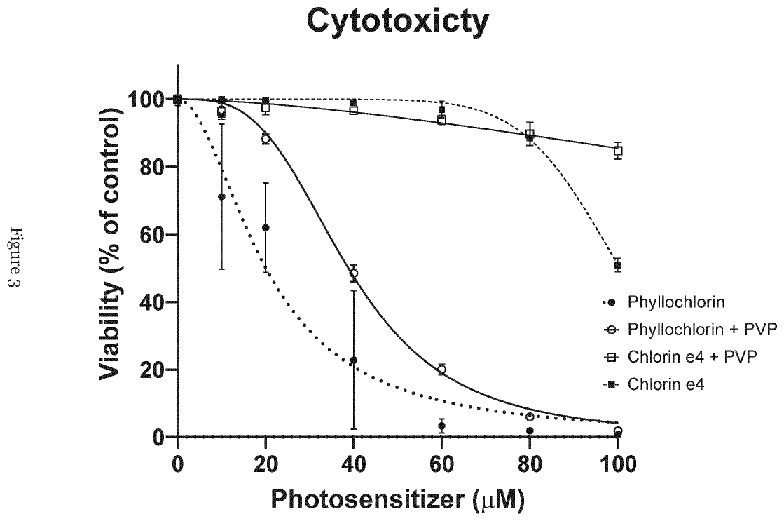

In the absence of PVP, cells treated with chlorin e4 disodium up to 80 μM remained >80% viable after 24 hours. With addition of PVP, viability was retained at 100 μM. Cells treated with phyllochlorin in the absence of PVP had viability >80% even at 10 μM; however, with the addition of PVP, cells retained >80% viability at concentrations up to 25 μM (FIG. 3). The addition of PVP therefore reduced inherent toxicity of both compounds, with increased solubility resulting in an improved tolerance of cells to phyllochlorin.

Surprisingly phyllochlorin with added PVP has lower dark toxicity than phyllochlorin without added PVP resulting in a better therapeutic index.

Phototoxicity

SKOV3 cells were seeded in 96-well black wall plates (Greiner #655090) at a cell density of 5000 cells in 100 μl culture medium per well. On reaching ˜60% confluence, so media was aspirated and replaced with fresh media containing the relevant phyllochlorin analogue from 0-100 μM in DMSO. Cells were incubated for a further 24 hours, allowing uptake of phyllochlorin analogues.

To test for phototoxicity, cells incubated with phyllochlorin analogues (0-10 μM in DMSO) had culture media replaced after 24 hours (as above) and were then exposed to a 652 nm laser (Invion) with laser density at 50 mW/cm² for 5 mins (total 15 J/cm²). Following activation, cells were cultured for a further 24 hours. Media was then replaced with fresh media containing AlamarBlue, and % viable cells remaining assessed as above. Controls included cells treated with phyllochlorin analogues but not activated by laser light; cells without phyllochlorin analogue treatment but with laser light; and untreated controls. All measurements were made in quadruplicate.

For comparative purposes, phyllochlorin analogues were tested and compared against both chlorin e4 disodium and Talaporfin sodium, a clinically approved photosensitizer used in the photodynamic treatment of lung cancers. Phototoxicity IC90 values and dark toxicity IC10 values were calculated using a log[inhibitor]-vs normalized response dose curve with variable slope, using the formula Y=100/(1+(IC90/X)\hat{ }HillSlope (phototoxicity IC90) or Y=100/(1+(IC10/X)\hat{ }HillSlope (dark toxicity IC10).

Results—Phototoxicity of Phyllochlorin Compared to Chlorin e4 Disodium

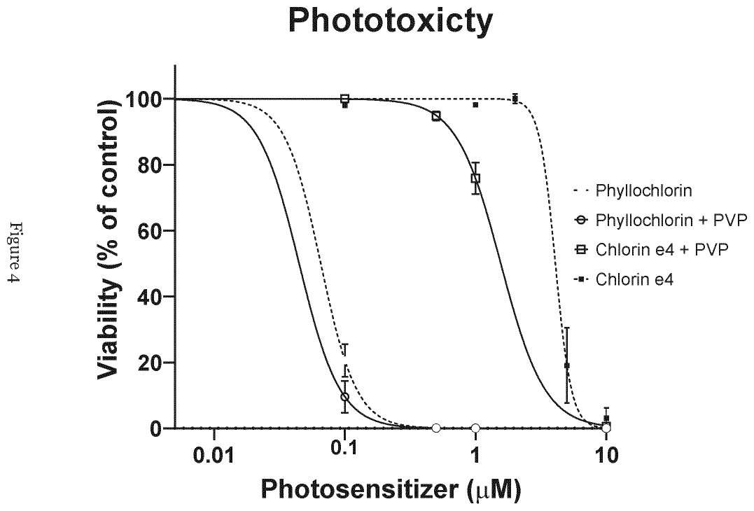

To assess phototoxicity, cells were treated with increasing concentrations of phyllochlorin or chlorin e4 disodium (0-10 μM in DMSO, prepared in the presence or absence of PVP) and subsequent laser (652 nm) activation. Chlorin e4 disodium was included to assess their comparative efficacy. IC50 values were estimated from fitted curves in each case. In the absence of PVP, chlorin e4 disodium returned an IC50 of 4.53 μM; this was improved by addition of PVP to 1.35 μM. By contrast, phyllochlorin had a calculated IC50 of 63.07 nM; addition of PVP reduced this to 53.85 nM (FIG. 4).

Surprisingly phyllochlorin is some 75 times more potent as a phototoxic agent than chlorin e4 disodium salt in ovarian cancer cells in head to head tests (0.06 μM to 4.53 μM). Phyllochlorin thus has substantially better phototoxicity than chlorin e4 disodium in vitro.

Toxicity Profile for Phyllochlorin Analogues

The phototoxicity and inherent cytotoxicity (i.e. “dark toxicity”) of several phyllochlorin analogues were assessed as previously using SKOV3 ovarian cancer cells. For comparative purposes, phyllochlorin analogues were compared against chlorin e4 disodium and Talaporfin sodium, a clinically approved photosensitizer used in the photodynamic treatment of lung cancers. Phototoxicity IC90 values and dark toxicity IC10 values were calculated using a log[inhibitor]-vs normalized response dose curve with variable slope, using the formula Y=100/(1+(IC90/X)\hat{ }HillSlope (phototoxicity IC90) or Y=100/(1+(IC10/X)\hat{ }HillSlope (dark toxicity IC10). The results are summarised in Table 1.

Both phyllochlorin and phyllochlorin monosodium salt (compound 1) displayed similar photo- and dark-toxicity profiles, as expected with conversion of the free acid to salt form in the sodium carbonate buffering system used in cell culture. Chlorin e4 disodium and Talaporfin sodium had substantially lower phototoxicity in vitro than all phyllochlorin analogues tested, with IC90 values in the μM range (versus nM range for phyllochlorin analogues). Compounds 3, 5, 6, 7 and 8 had greater phototoxicity than all other species tested with apparent IC90<100 nM in each case. Thus, phyllochlorin analogue species achieved ˜450 fold increase in phototoxicity compared to Talaporfin sodium, a clinically approved photosensitizer.

Therapeutic Index for Phyllochlorin Analogues

To evaluate the therapeutic potential of phyllochlorin analogues, the therapeutic index (TI) was calculated for each compound tested. TI provides a quantitative measurement to describe relative drug safety, by comparing the drug concentration required for desirable effects versus the concentration resulting in undesirable off-target toxicity. TI in each case was calculated using phototoxicity IC90 vs dark toxicity IC10.

The TI values for all compounds tested are provided in Table 1. Talaporfin sodium had the lowest calculated therapeutic index (TI=0.49) with chlorin e4 disodium only marginally better (TI=1.89), indicating that the potential therapeutic window for their use is small. The phyllochlorin analogues of the present invention had comparatively improved TIs with substantially greater phototoxicity (Table 1).

Thus, phyllochlorin analogues have a desirable therapeutic index that is better than a clinically applied photosensitizer. Moreover the greater phototoxicity of phyllochlorin analogues suggests their potential use at a greatly reduced dose in vivo. Phyllochlorin analogues therefore have an acceptable therapeutic profile for clinical application.

TABLE 1
Toxicity profile and therapeutic index for phyllochlorin analogues:

| | | | Cellular Toxicity | | |
|---|---|---|---|---|---|
| | | | | Dark | |
| | Singlet Oxygen | | | toxicity | |
| | Production (slope) | | Phototoxicity | IC10 | Therapeutic |
| Compound | ¹O₂ | ±SD | IC90 [μM] | [μM] | Index |
| Talaporfin | 736.7 | 60.7 | 22.83 | 11.10 | 0.49 |
| sodium | | | | | |
| Chlorin e4 | 998.2 | 128.4 | 21.32 | 40.23 | 1.89 |
| disodium | | | | | |
| Phyllochlorin | 1335.5 | 218.5 | 0.29 | 13.81 | 47.62 |
| free acid | | | | | |
| Compound 1 | 1445.0 | 50.1 | 0.35 | 21.96 | 62.74 |
| Compound 1a | 760.9 | 45.6 | 0.47 | 12.41 | 26.40 |
| Compound 1b | 841.7 | 7.3 | 1.40 | 22.13 | 15.81 |
| Compound 1c | 1229.0 | 7.7 | 0.21 | 58.01 | 276.24 |
| Compound 1d | 833.9 | 25.9 | 1.42 | 18.15 | 12.78 |
| Compound 1e | 938.8 | 13.6 | 1.79 | 20.73 | 11.58 |
| Compound 2 | 2141.0 | 36.0 | 0.26 | 39.62 | 152.38 |
| Compound 3 | 2054.0 | 275.8 | 0.08 | 20.66 | 258.25 |
| Compound 4 | 2191.0 | 55.3 | 0.12 | 16.41 | 136.75 |
| Compound 5 | 2385.0 | 47.3 | 0.05 | 20.82 | 416.40 |
| Compound 6 | 1700.0 | 41.3 | 0.06 | 9.49 | 158.17 |
| Compound 7 | 1494.0 | 4.2 | 0.05 | 14.27 | 285.40 |
| Compound 8 | 1314.0 | 192.3 | 0.06 | 12.55 | 209.17 |
| Compound 9 | 1396.0 | 24.3 | 0.19 | 70.24 | 369.68 |
| Compound 10 | 1691.0 | 17.5 | 0.16 | 14.94 | 93.38 |
| Compound 11 | 1434.0 | 23.6 | 0.50 | 29.00 | 58.00 |
| Compound 12 | 1121.0 | 11.2 | 0.18 | 28.36 | 157.56 |
| Compound 13 | 1423.0 | 19.6 | 0.12 | 13.02 | 108.50 |
| Compound 14 | 1293.0 | 48.5 | 0.17 | 22.79 | 134.06 |
| Compound 15 | 976.4 | 13.1 | 0.23 | 14.60 | 63.48 |
| Compound 16 | 884.1 | 40.6 | 0.25 | 28.64 | 114.56 |
| Compound 17 | 1265.0 | 13.1 | 0.22 | 76.27 | 346.68 |
| Compound 18 | 1060.0 | 18.3 | 0.10 | 32.30 | 323.00 |
| Compound 19 | 404.7 | 10.8 | 12.84 | 20.20 | 1.57 |
| Compound 20 | 1210.0 | 19.3 | 0.23 | 46.23 | 201.00 |
| Compound 21 | 423.4 | 7.4 | 4.77 | 8.17 | 1.71 |

Spectral Characteristics of Phyllochlorin Analogues

The phyllochlorin analogues prepared all displayed 4 absorption peaks, with a Soret band at 404±4 nm and Q bands in the green, red and far-red respectively. Chlorin e4 disodium and Talaporfin sodium had similar spectral characteristics. Complexation with a Zn²⁺ ion (compounds 19 and 21) resulted in spectral compression, with a red-shift in Soret and Q1 bands and a blue-shift in Q2 and Q3 bands.

Phyllochlorin Analogues Out-Perform Chlorin e4 Disodium and Clinically Approved Talaporfin Sodium as Photosensitizing Agents

The production of singlet oxygen (¹O₂), cellular phototoxicity and light-independent cytotoxicity (“dark toxicity”) of a number of phyllochlorin analogues were compared against chlorin e4 disodium and Talaporfin sodium (mono-L-aspartyl chlorin e6), the active ingredient of Laserphyrin™.

All phyllochlorin analogues displayed a similar or significantly enhanced rate of ¹O₂ generation and phototoxicity against SKOV3 cancer cells compared to both chlorin e4 disodium and Talaporfin sodium (Table 1). The exception to this rule occurred when Zn²⁺ was complexed with phyllochlorin analogues, which resulted in reduced ROS for compounds 19 and 21.

In some cases, the rate of ROS production was more than double that of either comparator (e.g. compound 5); and several compounds (e.g. compounds 3, 5, 6, 7 and 8) achieved cellular phototoxicities below 100 nM (Table 1). Non-specific cytotoxicity IC10 was similar to or better than Talaporfin sodium in all cases, suggesting that phyllochlorin analogues offer an improved therapeutic profile for use.

Phyllochlorin analogues therefore out-performed both chlorin e4 disodium and the clinically approved photosensitizer Talaporfin sodium, with (i) higher rates of singlet oxygen production; (ii) substantially greater phototoxicity against cancer cells; and (iii) similar or reduced non-specific cytotoxicity, suggesting phyllochlorin analogues should have lower off-target effects for therapeutic applications.

Metal Ions Alter the Spectral Properties and Activity of Phyllochlorin Analogues

Compound 18 was complexed with a Zn²⁺ ion to give compound 21, and their activities compared. Complexation with Zn²⁺ altered the spectral characteristics of compound 18, with a 14-16 nm red-shift evident in the Soret and Q1 bands; contrastingly, Q3 and Q4 bands were blue-shifted by 8 nm and 22 nm respectively. Inclusion of a Zn²⁺ ion also caused an ˜2.5 fold decrease in the rate of ROS production, and a major loss of phototoxic activity with an increase in non-specific dark toxicity (Table 1). Metal ions thus have a substantial impact on the activity and spectral characteristics of phyllochlorin and can be used to modulate activity as required.

Functionalisation with Saccharidyl Groups Enhances Phototoxicity

Phyllochlorin was functionalised with saccharidyl groups (glucose or mannose) using various configurations to give glucose-linked species (e.g. compounds 6 and 8) and mannose-linked species (e.g. compound 17). Whilst rate of ¹O₂ production was similar across all species, there was a marked increase in cellular phototoxicity for each saccharidyl-conjugated phyllochlorin analogue compared to compound 2 (Table 1). In particular, compounds 6 and 8 achieved a remarkable cellular phototoxicity IC90 of 0.06 μM, an approximately 4-fold increase compared to compound 2 (Table 1).

### Example 3—Cellular Uptake and Retention

Salt Substitution Does Not Alter Cellular Uptake or Retention In Vitro

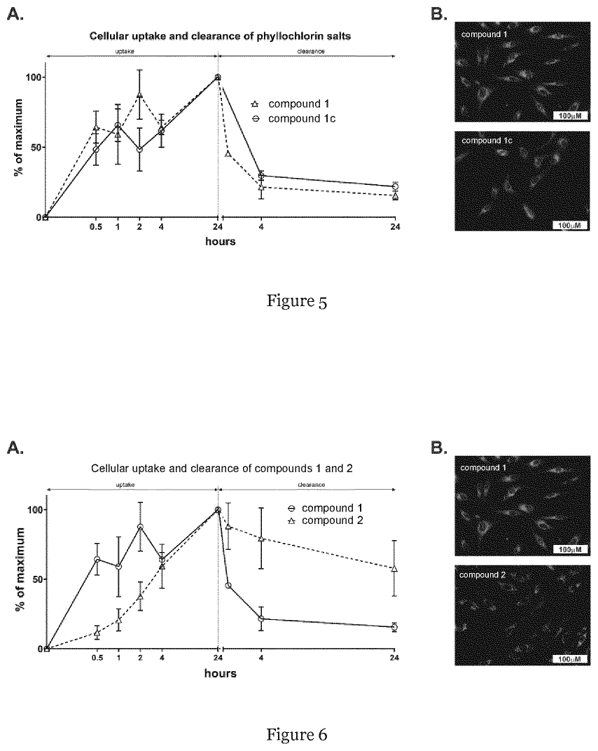

Based on the increased phototoxicity of compound 1c versus compound 1, it was assessed whether this salt substitution alters cellular uptake or retention over time. SKOV3 cells were incubated for up 24 hours in the presence of either compound 1 or 1c (as previously described). At defined time intervals (FIG. 5) the media was aspirated, replaced with sterile PBS, and cells imaged for fluorescence (Ex 405 nm/Em 660 nm) using a Cytation 3 Multimode Plate Reader (Biotek). After 24 hours the media was aspirated, replaced with fresh media and the loss of cellular fluorescence similarly monitored. All images (minimum 100 cells per time point) were then quantitatively analysed (Gen5.0 software) and an average fluorescence/cell/time point determined. Average fluorescence measurements were normalised against the maximum average value measured for each photosensitizer, and plotted as percent total fluorescence (mean±SD) overtime.

The relative uptake of compounds 1 and 1c, and their clearance from cells over 24 hours is shown in FIG. 5. There was no significant difference in either the uptake or clearance between the two salt forms (FIG. 5A). There was also no obvious difference in cellular localisation in either case (FIG. 5B), suggesting that the choline substitution did not affect cellular affinity or uptake of phyllochlorin.

Therefore, salt substitutions in phyllochlorin can alter its activity against cancer cells; and in particular, phyllochlorin choline salt (compound 1c) had an unexpectedly improved performance relative to other salts, despite no apparent change in ROS production rate or cellular uptake.

Compound 2 Exhibits Prolonged Cellular Retention

The uptake and retention of compounds 1 and 2 was compared in SKOV3 cancer cells over a 48 hour period. SKOV3 cells were incubated for up 24 hours in the presence of either compound 1 or 2 (as previously described). At defined time intervals (FIG. 6) the media was aspirated, replaced with sterile PBS, and cells imaged for fluorescence (Ex 405 nm/Em 660 nm) using a Cytation 3 Multimode Plate Reader (Biotek). After 24 hours the media was aspirated, replaced with fresh media and the loss of cellular fluorescence similarly monitored. All images (minimum 100 cells per time point) were then quantitatively analysed (Gen5.0 software) and an average fluorescence/cell/time point determined. Average fluorescence measurements were normalised against the maximum average value measured for each photosensitizer, and plotted as percent total fluorescence (mean±SD) over time.

The relative uptake of compounds 1 and 2, and their clearance from cells over 24 hours is shown in FIG. 6. There was no significant difference in the rate of uptake between the two compounds. However, compound 2 was retained at significantly higher levels in cells after 24 hours compared to compound 1, for which 80% of fluorescence was lost within 4 hours; by contrast, after 24 hours >50% of compound 2 remained within cells (FIG. 6). Compound 2 also displayed a distinctly punctate distribution in cells, suggesting a vesicular localisation different to that of compound 1 (FIG. 6B).

Functionalisation with Saccharidyl Groups Enhances Cellular Uptake

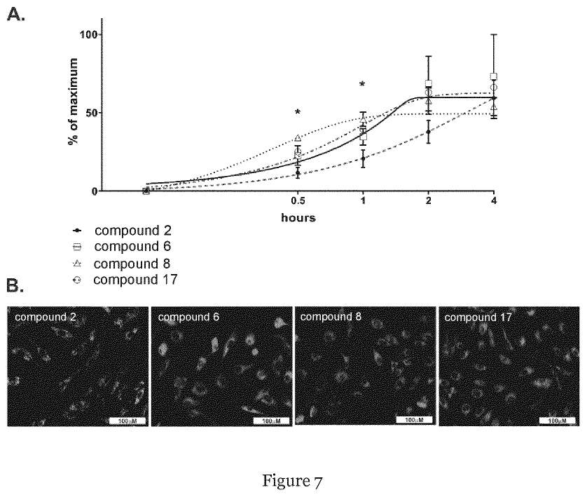

Phyllochlorin was functionalised with saccharidyl groups (glucose or mannose) using various configurations, and the effects on rate of uptake by cells assessed over a 4 hour incubation period as above. The rate of uptake of saccharidyl-conjugated phyllochlorin analogues exceeded that of unconjugated compound 2 (examples shown in FIG. 7); after 2 hours, saccharidyl-conjugated phyllochlorin analogues (compounds 6, 8 and 17) had reached 57-68% of their maximal uptake compared to 37% for compound 2 (FIG. 7A). By 4 hours total uptake was similar, and there was no apparent difference in cellular distribution between glucose (compounds 6 and 8) and mannose (compound 17) linked species (FIG. 7B). Of note, functionalisation also increased relative solubility resulting in a diffuse distribution pattern more similar to compound 1 than compound 2 (FIG. 7B).

### Example 4—Phyllochlorin Analogues are Active Against Multiple Cancer Cell Types

The activity of phyllochlorin analogues against multiple cancer cell types was assessed as above, using compounds 3 and 6 as examples. Phototoxicity is reported in Table 2. Compounds 3 and 6 showed exceptional activity against multiple cancer cell types, with phototoxicity IC90 as low as 0.03 μM in some cases (Table 2). These data demonstrate the potential of phyllochlorin analogues for use in pan-cancer therapies.

TABLE 2
Pan-cancer activity of compounds 3 and 6.

| | | Compound 3 | | Compound 6 | |
|---|---|---|---|---|---|
| | | Phototoxicity | Cytotoxicity | Phototoxicity | Cytotoxicity |
| Cell Line | Cancer Type | IC90 [μM] | IC10 [μM] | IC90 [μM] | IC10 [μM] |
| OVCAR3 | ovarian | 0.17 | 29.97 | not tested | |
| | adenocarcinoma | | | | |
| SKOV3 | ovarian clear cell | 0.11 | 20.66 | 0.19 | 9.49 |
| CaOV3 | ovarian | 0.05 | 5.43 | not tested | |
| | adenocarcinoma | | | | |
| A549 | lung | 0.20 | 4.22 | 0.14 | 29.52 |
| | adenocarcinoma | | | | |
| HH | T cell lymphoma | 0.11 | 14.80 | 0.03 | 10.25 |
| DLD-1 | colorectal | 0.36 | 42.28 | 0.33 | 9.20 |
| | adenocarcinoma | | | | |
| HEK293 | renal carcinoma | 0.22 | 8.11 | 0.20 | 8.16 |
| MDA- | breast | 0.07 | 6.00 | 0.07 | 13.25 |
| MB-468 | adenocarcinoma | | | | |
| | (triple negative) | | | | |
| LP9 | immortalised | 0.04 | 18.01 | 0.03 | 19.63 |
| | mesothelial cells | | | | |

| | | | | |
|---|---|---|---|---|
| B16F10 | melanoma | not tested | 0.64 | 22.85 |

### Example 5—Phyllochlorin Analogues are Non-Toxic and Localise to Tumour Tissues In Vivo

The potential application of compound 6 as a representative phyllochlorin species was explored in vivo. Wild type mice (Balb/C and C57BL/6) received compound 6 by intravenous or intraperitoneal injection, with doses ranging from 0.5 to 5 mg/kg. There were no adverse toxic events observed at any dose, demonstrating a good safety profile in agreement with in vitro data.

Tumour localisation and retention was explored using two models.

Model 1 used Balb/C wild type mice implanted subcutaneously with 10⁴×4T1 murine breast cancer cells. When tumours reached a palpable size (˜3-6 mm diameter) mice received compound 6 by either oral gavage (ORAL: 2.5 mg/kg), intravenous injection (IV: 1.0 mg/kg), or intratumoral injection (IT: 1.0 mg/kg in 501). To establish compound 6 localisation, mice were imaged using an IVIS Lumina III In Vivo Imaging System (Perkin Elmer) to detect fluorescence at 660 nm when illuminated under blue (400-460 nm) light.

Model 2 used C57BL/6 wild type mice implanted intraperitoneally with 1×10⁶ ID8 murine ovarian cancer cells. Following a 4 week incubation (at which point metastatic peritoneal disease with macroscopically visible tumour deposits was established), mice received compound 6 by either oral gavage (ORAL: 2.5 mg/kg), intravenous injection (IV: 1.0 mg/kg), or intraperitoneal injection (IP: 1.0 mg/kg). Quantitative imaging was performed as above; however, due to the disseminated nature of ovarian cancer, imaging was performed at autopsy, using blue light (400 nm) illumination to visualise red fluorescence associated with compound 6 in tumour deposits.

In all cases, compound 6 was specifically retained in tumour tissues for at least 24 hours following administration; whilst clearance from healthy tissues was typically achieved within 4-16 hours dependent on the tissue type. Timing and accumulation varied according to the model and administration route as below.

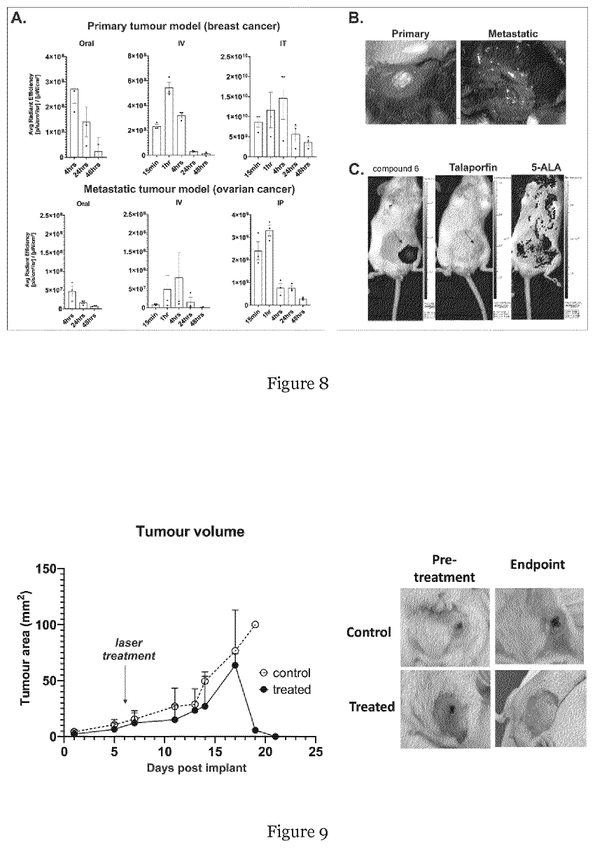

Oral administration: In primary subcutaneous tumours (model 1), peak levels of compound 6 occurred 4 hours post-administration and remained above background for at least 24 hours (FIG. 8A). A similar pattern was observed in metastatic peritoneal tumours (model 2) (FIG. 8A).

IV administration: In primary subcutaneous tumours (model 1), compound 6 was identified immediately after injection and peaked at 1 hour post-administration (FIG. 8A). Fluorescence was retained for at least 4 hours post-administration, and became undetectable at 24 hours post-injection. In metastatic peritoneal tumours (model 2), tumour distribution peaked at 4 hours post-injection. Compound 6 was retained for at least 24 hours following injection (FIG. 8A).

Intratumoral administration (model 1 only): IT delivery of compound 6 directly into tumour tissue resulted in strong localisation and retention in tumour tissue for at least 48 hours (FIG. 8A).

Intraperitoneal administration (model 2 only): IP administration resulted in rapid and highly selective localisation of compound 6 into metastatic nodules in vivo (FIG. 8A).

Compound 6 fluorescence was also readily imaged under blue light, and was visible to the naked eye (FIG. 8B). At autopsy, primary tumours were visualised as bright red mass (FIG. 8B, left). In mice with metastatic disease, multiple deposits could be seen on peritoneal surfaces and particularly extended to the liver, diaphragm and intestine, and contiguous peritoneal mass, consistent with ovarian cancer (FIG. 8B, right).

In a second experiment, the localisation of compound 6 at 24 hours post-administration was compared to that of Talaporfin sodium and 5-Aminolevulinic acid (5-ALA), with all compounds injected at 0.1 mg/kg IT. Fluorescence measurements were made using an IVIS Lumina III instrument (as above). Compound 6 localised strongly to tumour, with no obvious involvement of surrounding non-tumour tissue (FIG. 8C). By contrast, both Talaporfin sodium and 5-ALA could not be accurately detected at 24 hours; and in each case (particularly for 5-ALA) a diffuse, non-localised pattern of distribution was apparent (FIG. 8C).

Compound 6 thus specifically localised and retained in tumour tissues, and could be administered via multiple clinically relevant routes. Localisation of compound 6 was superficially superior to comparable photosensitizers already in clinical use. Moreover, the intrinsic fluorescence of compound 6 when illuminated under blue light could be used to more accurately identify or define tumour deposits and cancerous tissue margins in situ.

### Example 6—Phyllochlorin Analogues Regress Established Tumour Deposits In Vivo

Balb/C wild type mice with established 4T1 orthotopically implanted breast tumours were administered compound 6 by IT injection. At 24 hours post-injection (allowing compound 6 to clear from non-tumour tissues), mice were placed in clear perspex cages and illuminated (whole body) under red (660 nm) light for a period of 20 minutes. Light (660 nm) was delivered at an intensity of 100 mW/cm² for 20 mins continuously, to achieve a total light dose of 120 J/cm². Tumour size and appearance was subsequently monitored for a total of 2 weeks.

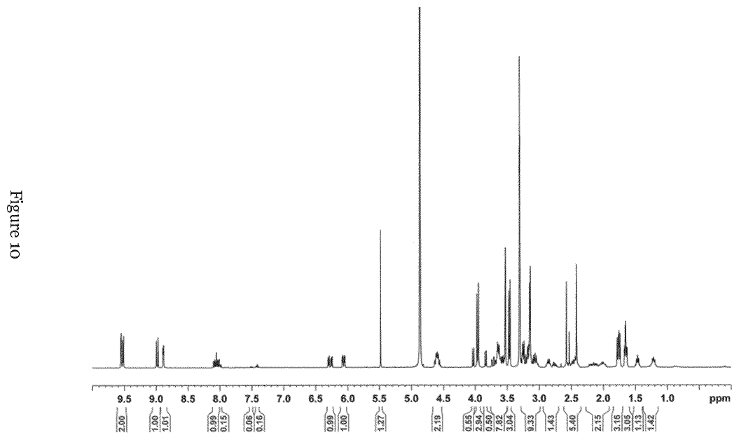

In mice that received compound 6 alone, tumours grew rapidly and mice reached endpoint within 13 days (FIG. 9). By contrast, in mice that received compound 6 plus laser treatment, tumours had regressed to an undetectable level after 14 days (FIG. 9). There was no evidence of regrowth even after 3 weeks (not shown), nor was there visible scarring following treatment (FIG. 9, right).

Compound 6 is thus an effective photosensitizer for use in photodynamic therapy, and results in complete tumour regression with no evidence of scarring of tissue damage.

### Example 7—Phyllochlorin is Non-Toxic when Injected into Mice

To assess acute toxicity, phyllochlorin or chlorin e4 disodium (each prepared with 1% PVP in PBS+20 mM HEPES) were administered at a dose of 5 mg/kg to C57BL/6 mice by intraperitoneal injection.

As PBS buffer (pH 7.4) is used in the experiments and the carboxylic acid in phyllochlorin (and the same carboxy group in other related chlorins) is estimated to have a pKa of less than 5, phyllochlorin in the PBS buffer will exist predominantly as the sodium and potassium salts with very little to no free acid.

Mice were observed for 30 mins for any clinical signs of acute toxicity (e.g. squinting, piloerection, loss of motility, general behaviour). No side effects were noted following administration of either compound, suggesting that phyllochlorin delivered at a dose of 5 mg/kg is safe for injection.

It will be understood that the present invention has been described above by way of example only. The examples are not intended to limit the scope of the invention. Various modifications and embodiments can be made without departing from the scope and spirit of the invention, which is defined by the following claims only.

1.-56. (canceled)

57. A compound of formula (I) or a complex of formula (II): 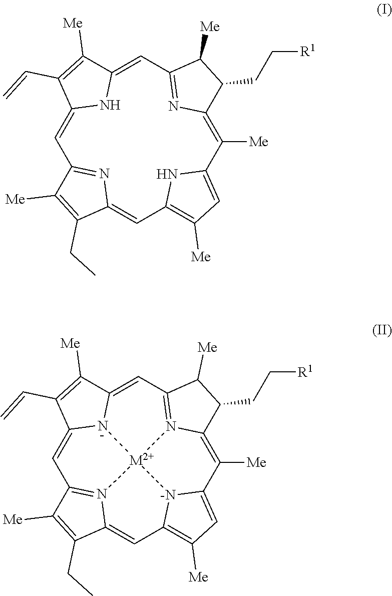
wherein
—R¹ is selected from —C(O)—OR³, —C(O)—SR³, —C(O)—N(R³)₂, —C(S)—OR³, —C(S)—SR³ or —C(S)—N(R³)₂;
—R³, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y or —R^{α}—[R⁶]Y;
—R^{α}—, each independently, is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P or Se in its carbon skeleton;
—R⁵, each independently, is selected from C₁-C₄ alkyl, C₁-C₄ haloalkyl, halo, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
—R⁶ is —[NC₅H₅] optionally substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or O—(CH₂CH₂O)ₙ—CH₃ groups;
n is 1, 2, 3 or 4;
Y is a counter ion;
X is a halo group;
M²⁺ is a metal ion;
or a pharmaceutically acceptable salt thereof;
provided that the compound is not:
(1) phyllochlorin free acid; or
(2) phyllochlorin methyl ester.

58. The compound or complex according to claim 57, wherein each —R^{α}— is independently selected from C₁-C₆ alkylene.

59. The compound or complex according to claim 57, wherein at least one —R³ is selected from —R^{α}—OR^{β}, —R^{α}—SR^{β}, —R^{α}—S(O)R^{β} or —R^{α}—S(O)₂R^{β}, and —R^{β} is a saccharidyl group.

60. The compound or complex according to claim 59, wherein —R^{β} is a saccharidyl group selected from:

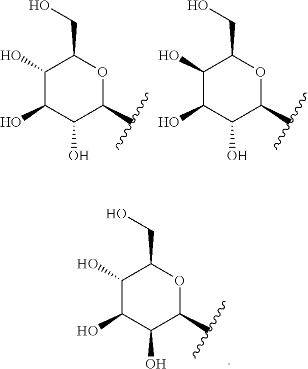

61. The compound or complex according to claim 60, wherein the saccharidyl group is:

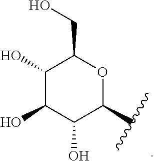 [SMILES: *[C@@H]1O[C@H](CO)[C@@H](O)[C@H](O)[C@H]1O]

62. The compound or complex according to claim 59, wherein —R^{β} is a saccharidyl group selected from: 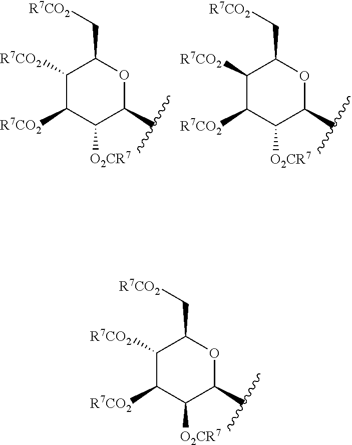
wherein —R⁷ is selected from C₁-C₄ alkyl.

63. The compound or complex according to claim 62, wherein —R⁷ is methyl.

64. The compound or complex according to claim 57, wherein —R¹ is —C(O)—N(R³)₂.

65. The compound or complex according to claim 64, wherein —R¹ is —C(O)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl.

66. The compound or complex according to claim 57, wherein the compound or complex is: 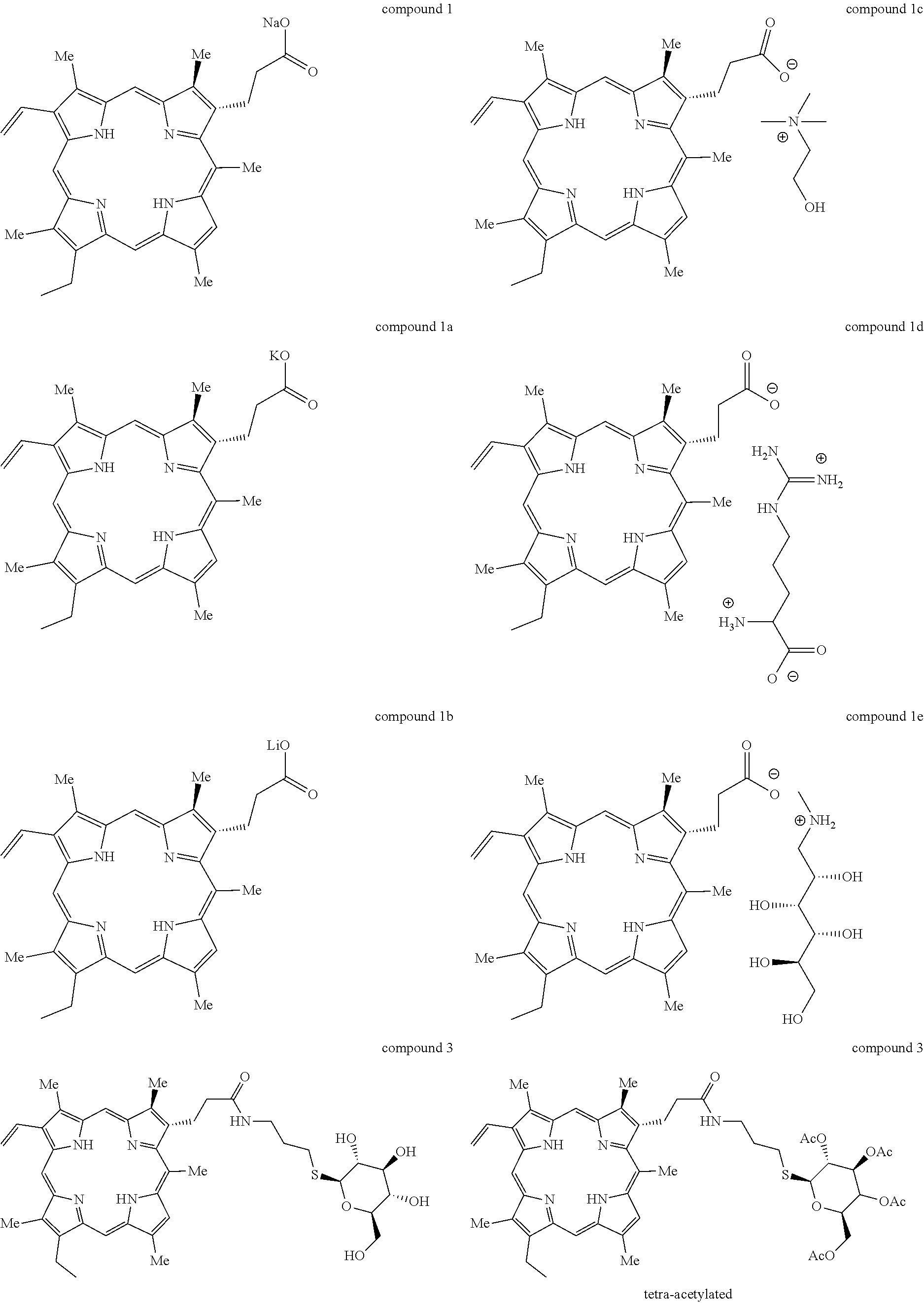 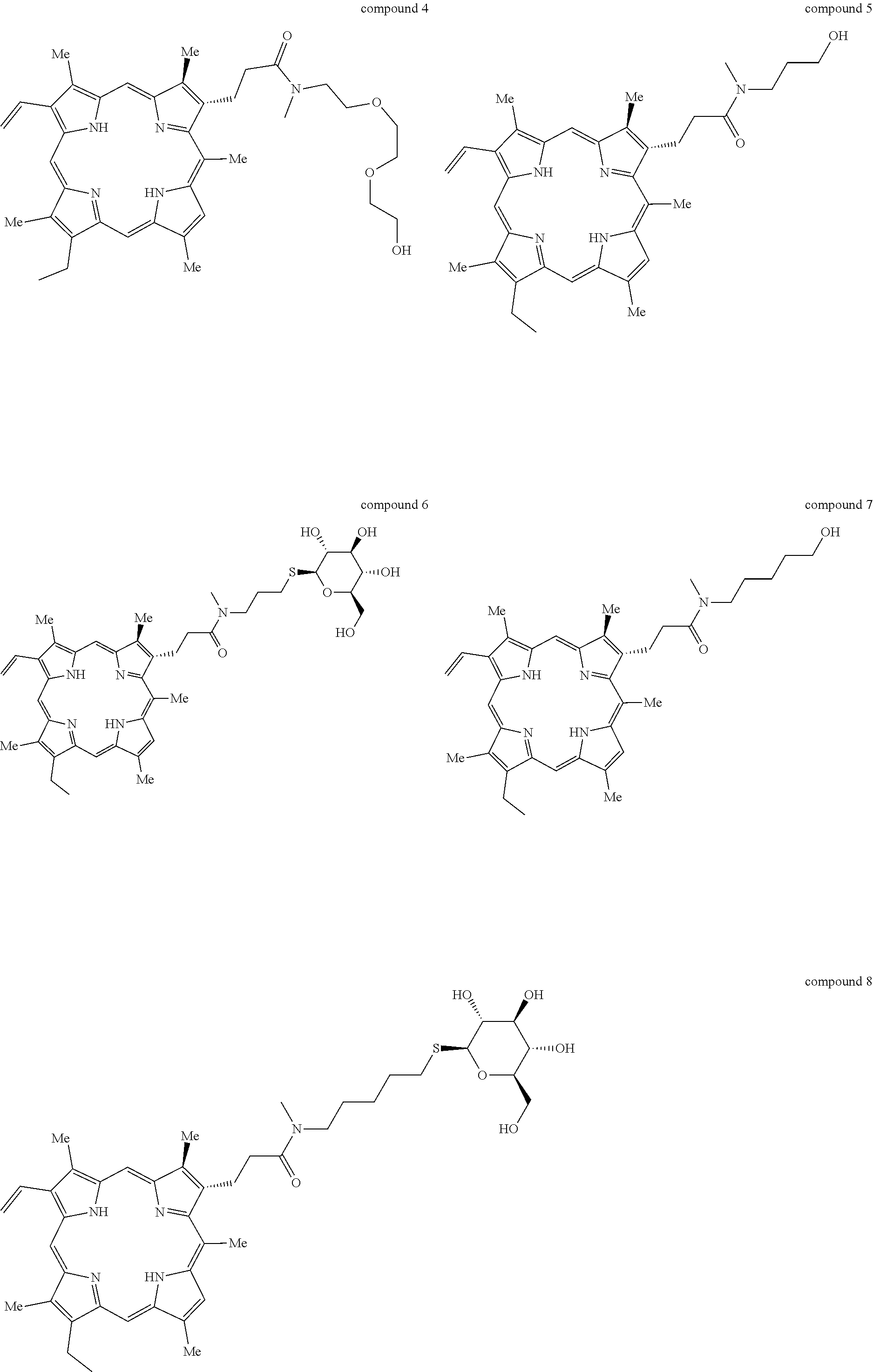 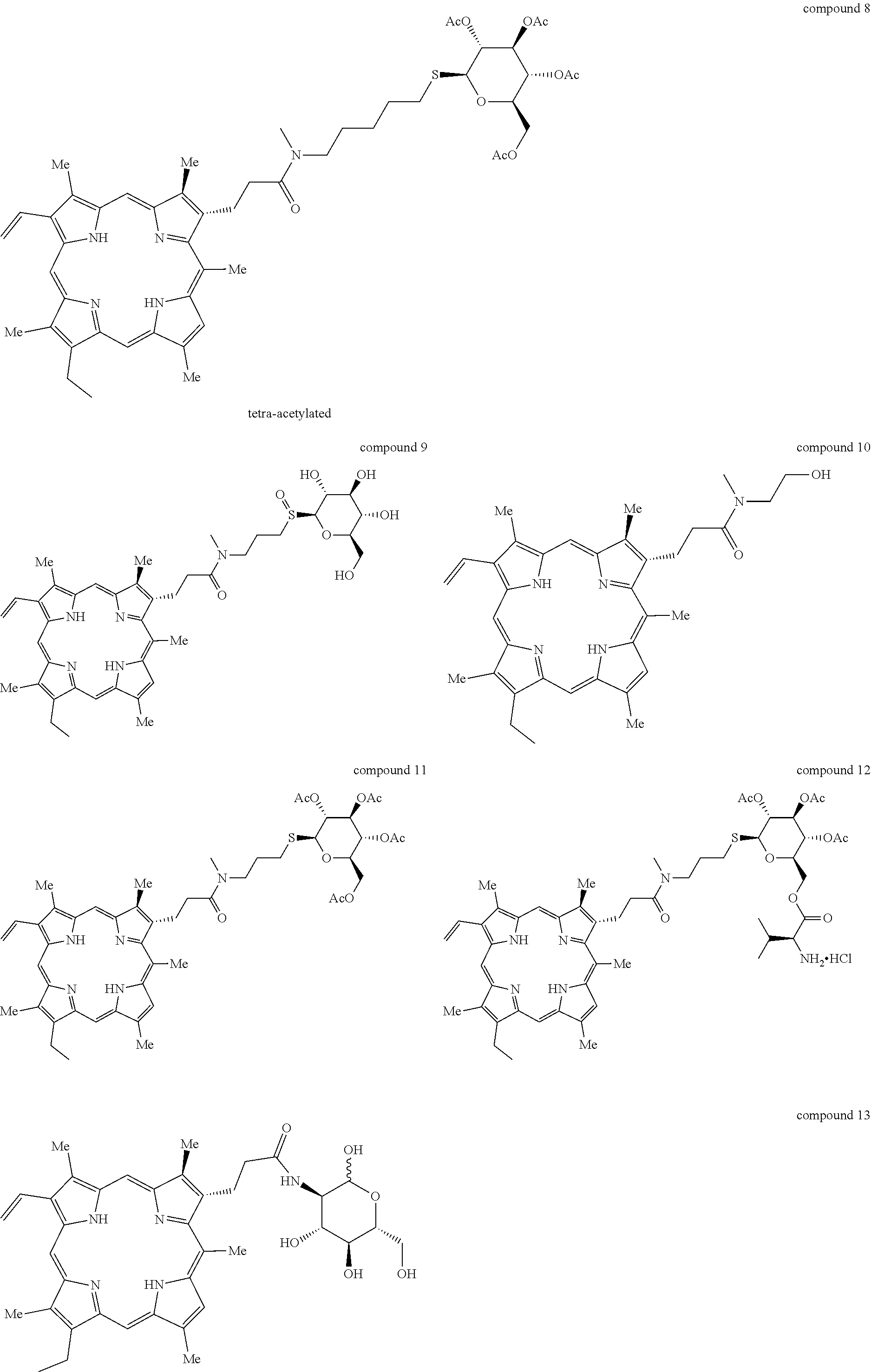 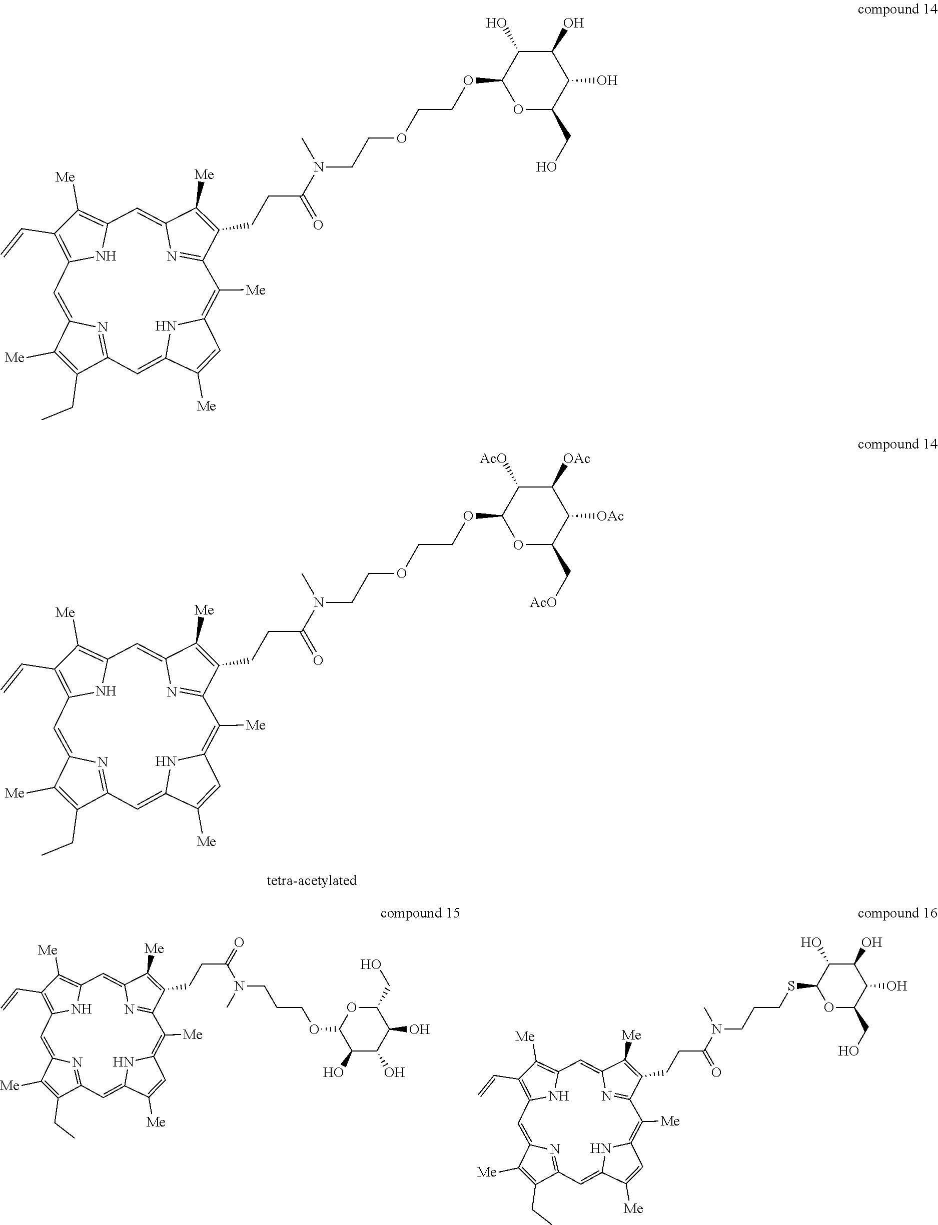 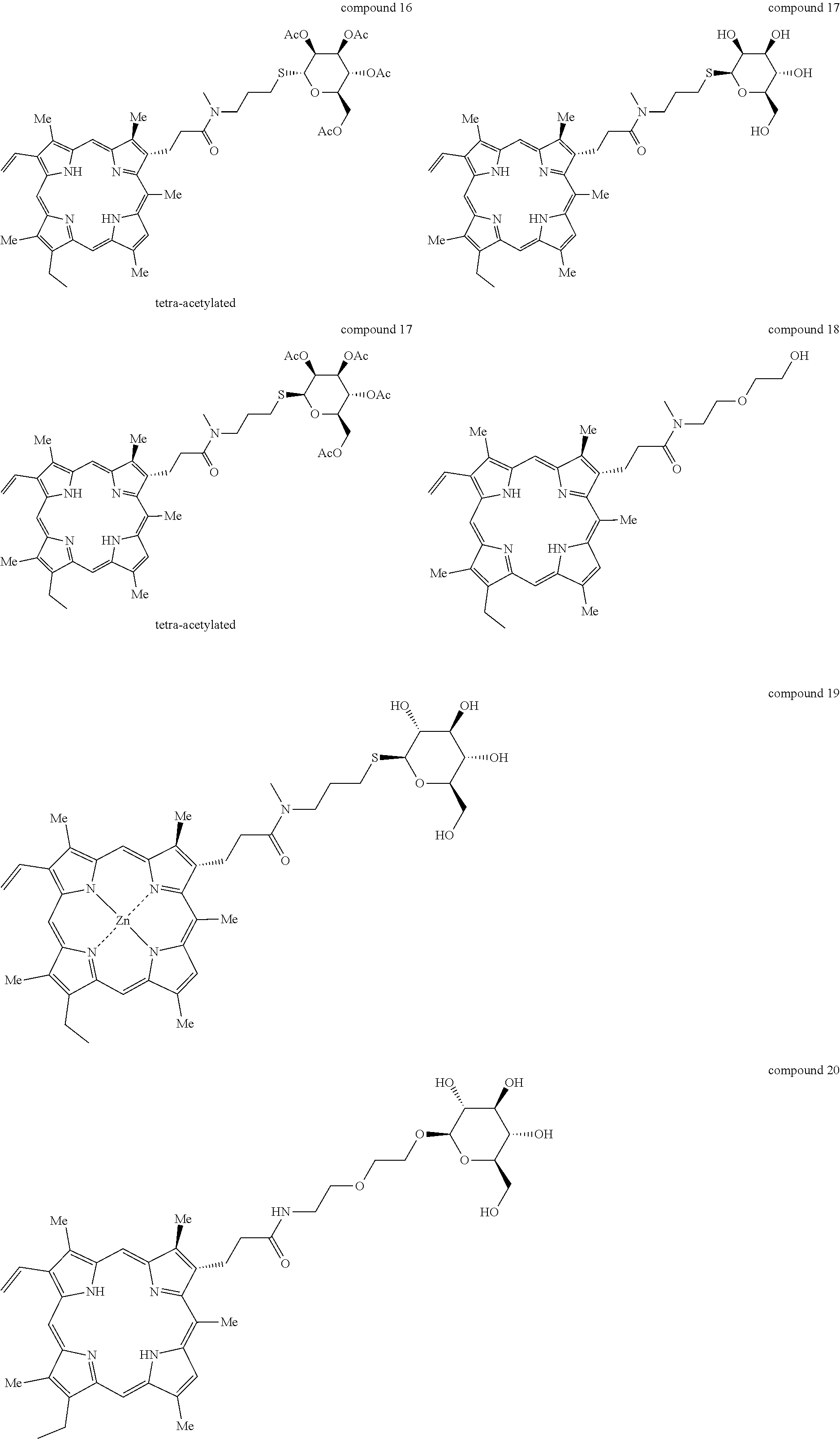 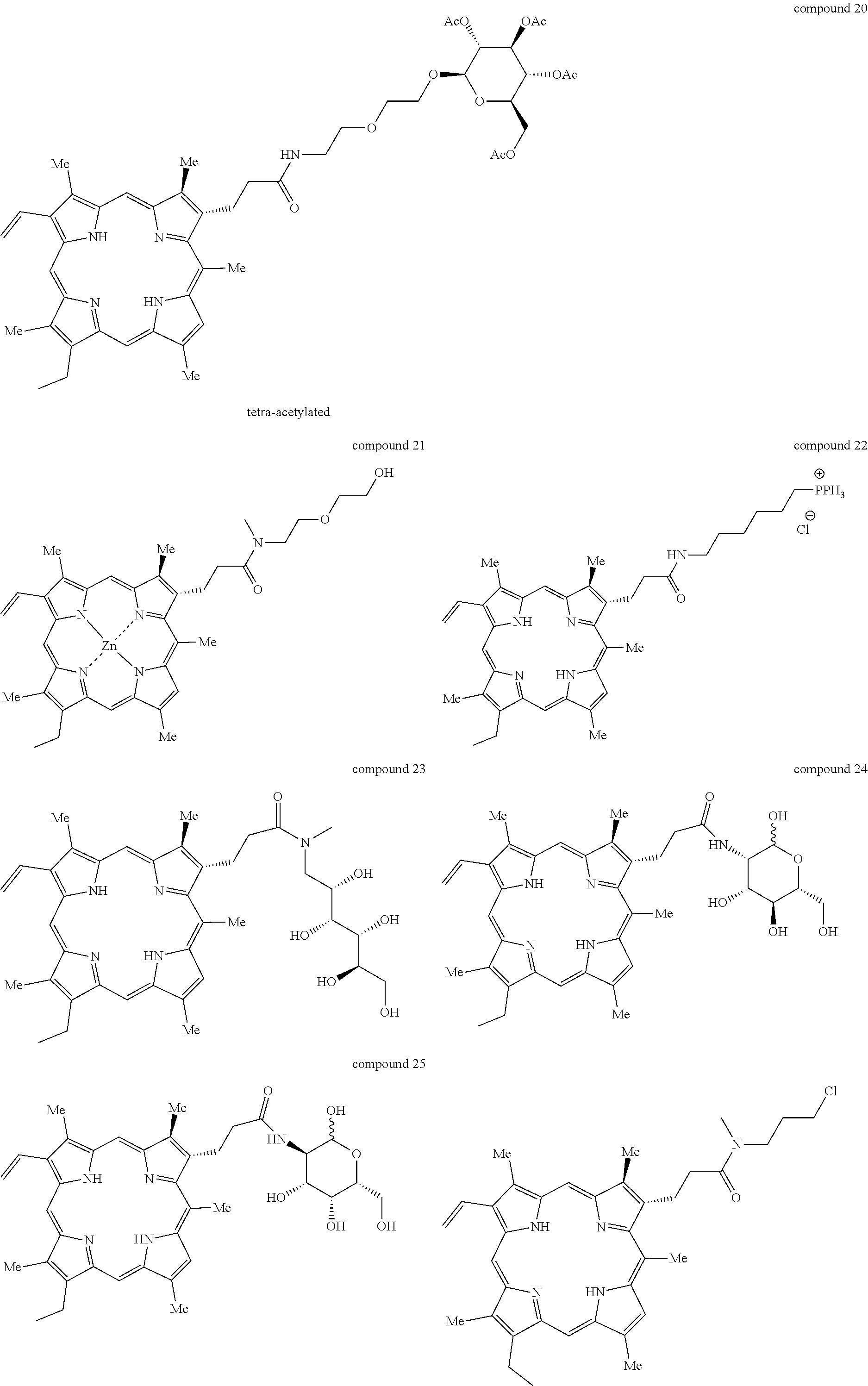 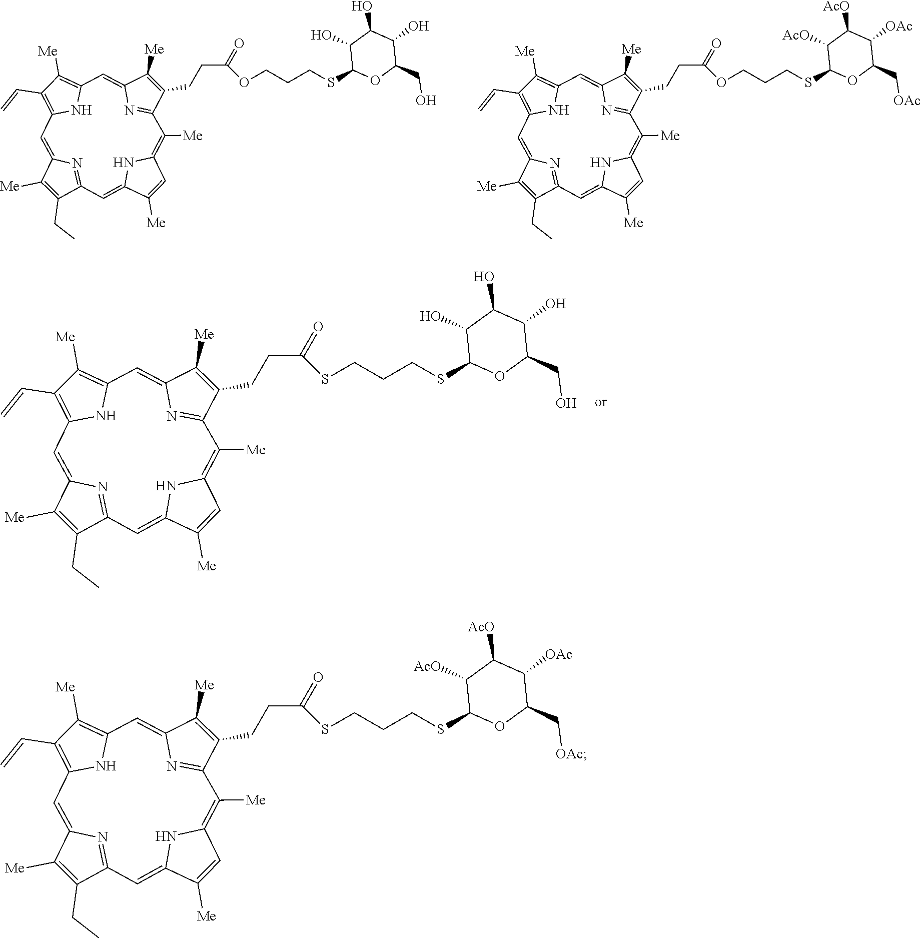
or a complex or a pharmaceutically acceptable salt thereof.

67. The compound or complex according to claim 57, wherein the compound is phyllochlorin in the form of a pharmaceutically acceptable salt.

68. The compound or complex according to claim 57, wherein the compound is phyllochlorin sodium, potassium, lithium, choline, arginine or meglumine.

69. A pharmaceutical composition comprising a compound or complex according to claim 57 and a pharmaceutically acceptable carrier or diluent.

70. The pharmaceutical composition according to claim 69, further comprising polyvinylpyrrolidone.

71. The pharmaceutical composition according to claim 69, further comprising an immune checkpoint inhibitor.

72. The pharmaceutical composition according to claim 71, wherein the immune checkpoint inhibitor is selected from Pembrolizumab, Nivolumab, Cemiplimab, Atezolizumab, Avelumab, Durvalumab or Ipilimumab.

73. The pharmaceutical composition according to claim 69, wherein the pharmaceutical composition is in a form suitable for oral, parenteral (including intravenous, subcutaneous, intramuscular, intradermal, intratracheal, intraperitoneal, intratumoral, intraarticular, intraabdominal, intracranial and epidural), transdermal, airway (aerosol), rectal, vaginal or topical (including buccal, mucosal and sublingual) administration.

74. The pharmaceutical composition according to claim 73, wherein the pharmaceutical composition is in a form suitable for oral or parenteral administration.

75. A method of treating atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas; the method comprising administering a therapeutically effective amount of a compound or complex to a human or animal, wherein the compound or complex is a compound of formula (I) or a complex of formula (II): 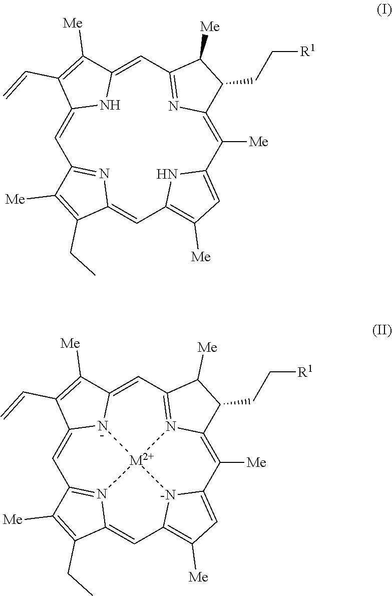
wherein
—R¹ is selected from —C(O)—OR³, —C(O)—SR³, —C(O)—N(R³)₂, —C(S)—OR³, —C(S)—SR³ or —C(S)—N(R³)₂;
—R³, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y or —R^{α}—[R⁶]Y;
—R^{α}—, each independently, is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P or Se in its carbon skeleton;
—R⁵, each independently, is selected from C₁-C₄ alkyl, C₁-C₄ haloalkyl, halo, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
—R⁶ is —[NC₅H₅] optionally substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
n is 1, 2, 3 or 4;
Y is a counter ion;
X is a halo group;
M²⁺ is a metal ion;
or a pharmaceutically acceptable salt thereof.

76. The method according to claim 75, wherein the method is a method of treating:
(a) a human or animal disease characterised by benign or malignant cellular hyperproliferation or by areas of neovascularisation; and/or
(b) a benign or malignant tumour; and/or
(c) early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

77. A method of photodynamic therapy or cytoluminescent therapy of a human or animal disease, the method comprising administering a therapeutically effective amount of a compound or complex to a human or animal, wherein the compound or complex is a compound of formula (I) or a complex of formula (II): 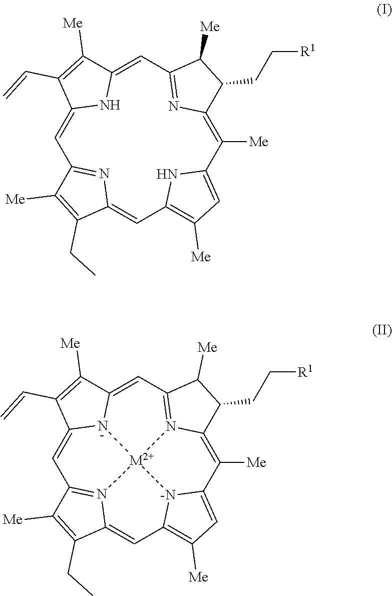
wherein
—R¹ is selected from —C(O)—OR³, —C(O)—SR³, —C(O)—N(R³)₂, —C(S)—OR³, —C(S)—SR³ or —C(S)—N(R³)₂;
—R³, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y or —R^{α}—[R⁶]Y;
—R^{α}—, each independently, is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P or Se in its carbon skeleton;
—R⁵, each independently, is selected from C₁-C₄ alkyl, C₁-C₄ haloalkyl, halo, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
—R⁶ is —[NC₅H₅] optionally substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
n is 1, 2, 3 or 4;
Y is a counter ion;
X is a halo group;
M²⁺ is a metal ion;
or a pharmaceutically acceptable salt thereof.

78. The method according to claim 77, wherein the human or animal disease is:
(a) atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas; and/or
(b) characterised by benign or malignant cellular hyperproliferation or by areas of neovascularisation; and/or
(c) a benign or malignant tumour; and/or
(d) early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

79. A method of photodynamic diagnosis of a human or animal disease, the method comprising administering a diagnostically effective amount of a compound or complex to a human or animal, wherein the compound or complex is a compound of formula (I) or a complex of formula (II): 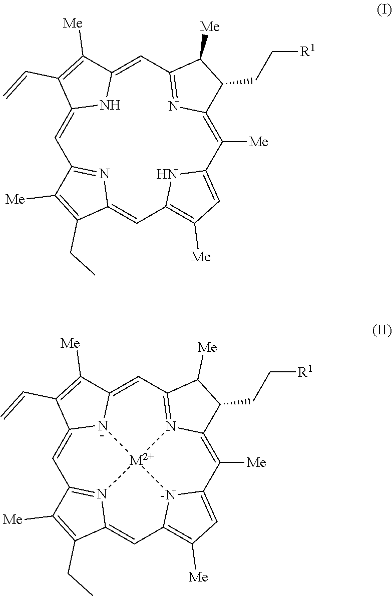
wherein
—R¹ is selected from —C(O)—OR³, —C(O)—SR³, —C(O)—N(R³)₂, —C(S)—OR³, —C(S)—SR³ or —C(S)—N(R³)₂;
—R³, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y or —R^{α}—[R⁶]Y;
—R^{α}—, each independently, is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P or Se in its carbon skeleton;
—R⁵, each independently, is selected from C₁-C₄ alkyl, C₁-C₄ haloalkyl, halo, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
—R⁶ is —[NC₅H₅] optionally substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
n is 1, 2, 3 or 4;
Y is a counter ion;
X is a halo group;
M²⁺ is a metal ion;
or a pharmaceutically acceptable salt thereof.

80. The method according to claim 75, wherein the human or animal is subjected to irradiation after the administration of the compound or complex, optionally wherein the irradiation is electromagnetic radiation with a wavelength in the range of from 500 nm to 1000 nm.

81. The method according to claim 77, wherein the human or animal is subjected to irradiation after the administration of the compound or complex, optionally wherein the irradiation is electromagnetic radiation with a wavelength in the range of from 500 nm to 1000 nm.

82. The method according to claim 79, wherein the human or animal is subjected to irradiation after the administration of the compound or complex, optionally wherein the irradiation is electromagnetic radiation with a wavelength in the range of from 500 nm to 1000 nm.

83. A pharmaceutical combination comprising:
(a) a compound or complex according to claim 1; and
(b) an immune checkpoint inhibitor.

84. The pharmaceutical combination according to claim 83, wherein the immune checkpoint inhibitor is selected from Pembrolizumab, Nivolumab, Cemiplimab, Atezolizumab, Avelumab, Durvalumab or Ipilimumab.
